(19) United States
(12) Patent Application Publication
(10) Pub. No.: US 2004/0076981 A1
(43) Pub. Date: Apr. 22, 2004

(54) Fungal gene cluster associated with pathogenesis

(76) Inventors: Olen Yoder, San Diego, CA (US); Barbara G. Turgeon, Ithaca, NY (US); Shun-Wen Lu, Ithaca, NY (US)

Correspondence Address: HALE AND DORR, LLP, 60 STATE STREET, BOSTON, MA, 02109

(21) Appl. No.: 10/432,422

(22) PCT Filed: Nov. 21, 2001
(86) PCT No.: PCT/US01/43381

(51) Int. Cl. C12Q 1/68; C07H 21/04; C12N 1/18; C12N 9/00; C07K 14/37

(52) U.S. Cl. 435/6; 435/69.3; 435/254.2; 435/320.1; 435/183; 530/350; 536/23.7

(57) ABSTRACT

Methods to identify orthologs of ungal CPS1 genes as well as fungal iron reductase and permease/and or MFS transporter genes, and uses thereof are provided.

### CROSS-REFERENCE TO RELATED APPLICATIONS

This application claims the benefit of the filing date of U.S. application Serial No. 60/252,649, filed on Nov. 22, 2000, and U.S. application Serial No. 60/252,732, filed Nov. 22, 2000, under 35 U.S.C. § 119(e), the disclosures of which are incorporated by reference herein.

### STATEMENT OF GOVERNMENT RIGHTS

The present invention was made with support from the United States Government (grant No. 96-35303-3198 from the USDA/NRI). The United States Government may have certain rights in the invention.

### FIELD OF THE INVENTION

The present invention relates to DNA molecules comprising fungal, e.g., Cochliobolus heterostrophus, genes from a peptide synthetase gene cluster, e.g., an iron reductase and/or a permease or major facilitator superfamily transporter, and uses thereof.

### BACKGROUND OF THE INVENTION

There are approximately 30 species included in the genus Cochliobolus, nearly all of which are pathogens of wild grasses or cereals (Yoder et al., In: The Mycota Vol. 5; Plant Relationships, Part A, Berlin: Springer-Verlag, Carroll, eds., pp. 145-166 (1997)). Cochliobolus heterostrophus represents the most widely distributed species in the genus and can be found in many tropical and subtropical areas in the world. As a natural pathogen of corn, C. heterostrophus causes a disease frequently called leaf spot of maize in the old literature (Drechsler, J. Agr. Res., 31:701 (1925); Drechsler, Phytopathol., 24:953 (1934); Yu, “Studies on Helminthosporium maydis,” 36:327 (1952)). In the United States, C. heterostrophus is usually found in the warmer southern states, thus, the disease is commonly known as Southern Corn Leaf Blight (Hooker, Ann. Rev. Phytopathol., 12:167 (1974)). For many years, Southern Corn Leaf Blight was only known as an endemic disease and was not considered to be major economic importance in the United States. But in 1970, it suddenly broke into a severe epidemic that destroyed 15% of the U.S. corn crop and caused losses estimated at more than $1 billion. This serious damage made Southern Corn Leaf Blight one of the most widely known crop diseases in the U.S.

Prior to the outbreak of the disease, only one race of C. heterostrophus (race O) was known in the field. In late 1969 when the disease became an epidemic, a new race of the fungus was identified from infected corn leaves collected in severely diseased areas. It was soon designated as race T because of its high virulence on T-cytoplasm corn and the ability to produce a phytotoxin called T-toxin, which specifically affects T-corn. In contrast, race O does not produce T-toxin and is mildly virulent on both T-cytoplasm and N-cytoplasm (normal cytoplasm) corn (Hooker et al., Plant Dis. Reptr., 54:1109 (1970); Scheifele, “Cytoplasmically Inherited Susceptibility to Diseases Related to Cytoplasmically Controlled Pollen Sterility in Maize,” 25:110 (1970); Smith et al., Plant Dis. Rep., 54:819 (1970); Yoder et al., Phytopathology 65:273 (1975); Yoder, In: Biochemistry and Cytology of Plant Parasite Interaction, New York, N.Y.: Elsevier, Tomiyama, eds., pp. 16-24 (1976); Yoder, Ann. Rev. Phytopathol., 18:103 (1980)). T-cytoplasm stands for Texas male sterile cytoplasm, a unique cytoplasm with a trait for maternally inherited male sterility, characterized by the failure to produce pollen (Levings, Science, 250:942 (1990)). T-cytoplasm corn was widely used for hybrid seed production and breeding to avoid hand or mechanical emasculation in the 1950s and the 1960s. It was the coexistence of large acreages of intensively planted T-cytoplasm corn and the sudden appearance of race T of C. heterostrophus that resulted in the epidemic of the disease in 1970. This discovery first opened the door to understanding pathogenesis by C. heterostrophus.

Early genetic analysis suggested that both T-toxin production and high virulence on T-cytoplasm corn are controlled by a single genetic locus defined as Tox1 (Leach et al., Physiol. Plant Pathol., 21:327 (1982)). This was demonstrated by crosses between race T and race O in which only parental phenotypes segregated in a 1:1 ratio (Tox+:Tox−); all T-toxin producing progeny are highly virulent on T-cytoplasm corn while all T-toxin nonproducing progeny are weakly virulent (Yoder et al., 1975, supra; Leach et al., 1982, supra). Further investigation by comparison of electrophoretic karyotypes and chromosome-specific DNA hybridizations indicated that Tox1 is tightly linked to a reciprocal translocation breakpoint and is associated with as much as a megabase of DNA (mostly highly repeated and A+T-rich) that is missing in race O (Bronson, Genome, 30:12 (1988); Tzeng et al., Genetics, 130:81 (1992); Chang et al., Genome, 39:549 (1996)). Surprisingly, recent analysis of several Tox mutants revealed that Tox1 is not a single locus but rather two loci, each on a different translocated chromosome (Yoder et al., In Host-Specific Toxin: Biosynthesis, Receptor and Molecular Biology, Tottori, Japan: Faculty of Agriculture, Tottori Univ., Kohmoto, eds., pp. 23-32 (1994); Turgeon et al., Can. J. Bot., 73:S1071 (1995)). These two Tox1 loci have been designated Tox1A and Tox1B (Yoder et al., 1997, supra). Two genes PKS1 and DEC1 have been cloned from the two loci respectively, both are required for biosynthesis of T-toxin and are found only in race T isolates of C. heterostrophus (Yang, “The Molecular Genetics of T-Toxin Biosynthesis by Cochliobolus heterostrophus,” Ph.D. Thesis, Cornell University (1995); Yang et al., Plant Cell, 8:2139 (1996); Rose et al., 8th Int. Symp. Mol. Plant-Microbe Int., Knoxville, p. J-49 (1996)).

Genetic analysis also suggested that T-toxin is required by C heterostrophus for its high virulence on T-cytoplasm corn. This hypothesis was first tested by the generation of induced T-toxin deficient mutants using different mutagenesis procedures. All mutants with a tight Tox⁻ phenotype cause disease symptoms that are indistinguishable from those caused by race O when tested on both T and N-cytoplasm corn, suggesting that T-toxin is indeed a virulence factor (Yang et al., 1992; Lu et al., Proc. Natl. Acad. Sci. USA, 91:12649 (1994); Rose et al. (1996), supra). This conclusion was firmly supported by the site-specific disruption of the PKS1 or DEC1 in the wild type race T genome; disruptants lost the ability to produce T-toxins and caused race O type symptoms on both T-com and N-com (Yang et al., 1996, supra; Rose et al., 1996, supra). These experiments have given a very clear resolution for the role of T-toxin in pathogenesis. They also implied that pathogenesis by C. heterostrophus must involve additional pathogenicity factors because race O which does not produce T-toxin and race T-derived Tox⁻ mutants are effective pathogens on corn.

A number of fungal molecules have been identified as general pathogenicity or virulence factors in several plant-pathogenic fungi (Yoder et al., J. Genet. 75:425 (1996)). These include potential penetration factors such as melanin (Guillen et al., Fungal Genet. Newsl., 41:41 (1994)), cutinase (Oeser et al., Mol. Plant-Microbe Int., 7:282 (1994)) and polygalacturonase and xylanase (Lyngholm et al., Fungal Genet. Newsl., 42:46 (1995)) or possible mechanisms involved in colonization such as phytotoxin detoxification (Schafer et al., Science, 246:247 (1989)) or components of signal transduction pathways. Although C. heterostrophus is known to produce a nonhost specific toxin called ophiobolin (or cochliobolin), a C₂₅ sesterterpenoid compound, which is toxic to many organisms, including plants, bacteria, fungi and nematodes, there is no evidence that ophiobolins are involved in pathogenesis by C. heterostrophus or other phytopathogenic fungi. No other pathogenesis-related toxins have been isolated from C. heterostrophus so far, but studies on closely related Cochliobolus species and other phytopathogenic fungi suggest that pathogenesis by this group of fungi also involves peptide toxins.

Four peptide phytotoxins (victorin, HC-toxin, AM-toxin, and enniatins) have been characterized as pathogenicity or virulence factors. They are all small cyclic peptides (4-6 residues), containing unusual amino acids or hydroxy acids, and they can be either host specific or non-host specific in terms of plant toxicity. A number of peptide phytotoxins are believed to be synthesized nonribosomally. Early in the 1960s, several biochemists working on the bacterial peptide antibiotics gramicidin and tyrocidine found that these polypeptides can be synthesized in RNAase-treated particle-free extracts of Bacillus brevis that are known to produce the same antibiotics; adding protein-synthesis inhibitors to the extracts does not affect this process. This indicated the existence of a peptide biosynthetic system in which ribosomes and mRNAs are not needed. Further studies revealed that in this system, peptides are synthesized on a protein-template and this template itself is a multifunctional enzyme or a complex of several such enzymes, collectively called peptide synthetases, catalyzing the biosynthetic process (Laland et al., Essays in Biochemistry 7:31 (1973); Lipmann, Adv. Microbiol. Physiol., 21:277 (1980)).

Peptide synthetases can catalyze biosynthesis of a variety of peptides. In terms of bioactivity, they can be antibiotics, enzyme inhibitors, plant or animal toxins and immunosuppressants (Stachelhaus et al., Journal of Biological Chemistry, 270:6163 (1995)). In terms of chemical structure, they can be either linear (i.e., ACV, the penicillin precursor and gramicidin) or cyclic (most are). The latter can be further classified into three subgroups: 1) The “standard” cyclic peptides (i.e., gramidicin S, tyrocidine, HC-toxin and cyclosporin); 2) cyclic lactones (i.e., destruxin); and 3) cyclic depsipeptides (i.e., beauvericin and enniatin). There have been over 300 different carboxy compounds that can be activated by peptide synthetases.

Although the first peptide synthetase, Gramicidin S synthetase, was purified and used for the cell-free synthesis of the peptide early in the 1960s (Tomino et al., Biochem, 6:2552 (1967)), the first bacterial peptide synthetase gene, tycA, which encodes the tyrocidine synthetase 1 in B. brevis, was not cloned until almost twenty years later (Marahiel et al., Mol. Gen. Genet. 201:1986(1985)). Since then, more than twenty peptide synthetase genes have been reported for both bacteria and filamentous fungi, but only fourteen have complete nucleotide sequences published. All are larger than 3.3 kb and range between 3.3-19.5 kb for bacterial genes and 9.445.8 kb for fungal ones. Interestingly, all fungal peptide synthetase genes reported lack introns, even the cyclosporin A synthetase gene simA, which has a 45.8 kb of open reading frame (the largest genomic ORF so far recorded). Although biosynthesis of bacterial peptides differs from that of fungal ones in terms of the number of multifunctional enzymes involved, the genes encoding these enzymes are similar to each other in both function and structure.

Comparison of nucleotide sequences reveals one or more highly conserved regions at certain positions in each peptide synthetase gene. These regions formerly called “amino acid activating domains” (Stachelhaus et al., 1995, supra), now called “amino acid activating modules” (Marahiel, Chem. Biol., 4:561 (1997)) consist of a set of domains (formerly called “modules”) believed to have specific functions such as recognition, activation and thioesterification of individual constituent amino or hydroxy acids, and in some cases methylation and racemation for modification of certain residues before incorporation into the peptide chain (Stachelhaus et al., 1995, supra). The most convincing evidence supporting this assignment is that in most cases, the number of conserved functional units in each gene or gene cluster is equal to the number of amino acids in the respective peptide. This one-for-one match is very clear between three of four fungal peptides and their biosynthetic genes. The total number of modules in three of four bacterial gene clusters also matches the number of amino acids in the respective peptides.

Sequence alignment of amino acid-activating modules reveals strictly conserved sequence motifs that contain active residues for module functions. These motifs are called “core sequences” (Marahiel, FEBS Lett., 307:40 (1992)). A minimal amino acid-activating module must contain six core sequences, whose functions (except for core 1) have been proposed based on mutational analysis of several peptide synthetases. Core sequences 1-5 are grouped into an amino acid adenylation domain and core 6 is a thioester formation domain (FIG. 1A). All bacterial peptide synthetase genes contain “type I modules,” the minimal amino acid activating modules which were previously called “type I domains” (Stachelhaus et al., 1995, supra). Two fungal genes, acvA and HTS1 also have this modular structure. In addition to the type I module, two fungal genes, esyn1 and simA, contain type II modules, in which an insertion (about 400 amino acids) is found between cores 5 and 6 of a normal type I module. This region contains a motif (VLE/DXGXGXG; SEQ ID NO:1), highly conserved in S-adenosyl-methionine (SAM)-dependent methyltransferases, hence, it is referred to as a N-methylation domain (FIG. 1A). Additional evidence for methyltransferase activity of this module is that the number and position of type II modules in esyn1, and simA exactly match that of N-methylated amino acids in ennatin and cyclosporin sequences (FIG. 1B).

Although the modular structure described above is highly conserved among most peptide synthetase genes, some variations have been found in the latest cloned peptide synthetase gene safB, which is the first gene in the saframycin Mx1 synthetase gene cluster (Pospiech et al., Microbiology 141:1793 (1995)). safB contains two type I amino acid activating modules. One module has all six highly conserved core sequences, but another, believed to activate alanine (the first amino acid in the linear tetrapeptide precursor of saframycin Mx1), lacks core 5 and has a weakly conserved core 1 (Pospiech et al., Microbiology, 142:741 (1996)) (FIG. 1A). This suggests that some of the motifs in the amino acid adenylation domain are dispensable or not critical for domain function. It also raises the possibility that other variations might be found in yet unknown peptide synthetase genes.

Although C. heterostrophus has been a model eukaryotic plant pathogen since the 1970s, most molecular genetic analyses conducted in this system have focused on production of the polyketide T-toxin by race T isolates of the fungus. Solid evidence now indicates that T-toxin is a host-specific virulence factor in Southern Core Leaf Blight (Yoder et al., J. Genet., 75:425 (1996); Yoder et al., 1997). It is clear, however, that C. heterostrophus needs additional factors, presumably general factors for pathogenesis to corn plants, since race O, which does not produce T-toxin, can be an effective corn pathogen. Attempts to identify additional general factors required by C. heterostrophus for pathogenesis have been unsuccessful.

Thus, what is needed is the isolation and characterization of additional fungal genes that control the biosynthesis of novel fungal molecules associated with pathogenesis, i.e., genes which are potential targets for the design of products that might interfere with the infection process, and vertebrate fungal orthologs of fungal peptide synthetase genes.

### SUMMARY OF THE INVENTION

The invention generally relates to an isolated nucleic acid molecule (polynucleotide), e.g., DNA or RNA, comprising a nucleic acid segment which encodes a gene product related to pathogenesis. In one embodiment of the invention, fungal genes which are related to pathogenesis are identified. An advantage of the present invention is that the genes described herein provide the basis to identify a novel fungicidal or mycocidal mode of action which permits rapid discovery of novel inhibitors of gene products that are useful as fungicides or mycocides. In addition, the invention provides isolated genes or gene products from fungi for assay development for inhibitory compounds with fungicidal or mycocidal activity, as agents which inhibit the function or reduce or suppress the activity of those gene products in fungi are likely to have detrimental effects on fungi, and are good fungicide or mycocide candidates. The present invention therefore also provides methods of using a polypeptide encoded by one or more of the genes of the invention or a cell expressing such a polypeptide to identify inhibitors of the polypeptide, which can then be used as fungicides to suppress the growth of pathogenic fungi. Pathogenic fungi are defined as those capable of colonizing a host and causing disease. Examples of fungal pathogens include plant pathogens such as Septoria trici, Ashbya gossypii, Stagenospora nodorum, Botryus cinera, Fusarium graminearum, Magnaporthe grisea, Cochliobolus heterostrophus, Colleetotrichum, Ustilago maydis, Erisyphe graminis, plant pathogenic oomycetes such as Pythium ultimum and Phytophthora infestans, as well as dimorphic fungal pathogens including Blastomyces, e.g., B. dermatitidis, Coccidioides, Histoplasna, e.g., H. capsulatum, or Paracoccidiodes, e.g., P. brasiliensis, Loboa, Malassezia, Rhodotorrula, Blastoschizomyces, Trichosporon, Saccharomyces, Cryptococcus including Cryptococcus neofomans, as well as human pathogens such as Candida albicans, and other pathogenic Candida, e.g., C. tropicalis, C. parapsolosis and C. guiettermondii, Coccidioidus imitis, and Aspergillus fumigatus, Sporothrix schenckii, pathogenic members of the Genera Epidermophyton, Microsporum and Trichophyton, Cladosporium (Xylohypha) trichoides, Cladosporium bantianum, Penicillium marnefii, Exophiala (Wangiella) dermatitidis, Fonsecaea pedrosoi and Dactylaria gallopava (Ochroconis gallopavum), and including mycogens. Preferred fungi for use with the agent identified by the method of the invention are Ascomycota.

In one embodiment of the invention, the invention relates to an isolated polynucleotide comprising a nucleic acid segment encoding an ortholog of a plant fungal CPS1, e.g., SEQ ID NO:3 from Cochliobolus which is a CoA ligase, or a nucleic acid segment encoding a gene product that modulates fungal iron metabolism, uptake, absorption of inorganic or organic ferric salts, e.g., a fungal iron reductase, permease or MFS transporter, e.g., a siderophore transporter, which genes maybe associated with CPS1 in a gene cluster. As described herein below, a gene from Coccidioidus imitis and Candida that is related to the CPS1 gene of Cochliobolus was identified, e.g., a nucleic acid sequence comprising an open reading frame comprising SEQ ID NO:46 which encodes SEQ ID NO:47 or the complement thereof. The CPS1 gene in Cochliobolus is present in a cluster of closely linked open reading frames, a cluster which is associated with virulence and/or pathogenicity, wherein CPS1 is representative of a novel class of adenylation domain-containing enzymes related to but distinct from nonribosomal protein synthetases (NRPSs). Thus, at least one of the genes in the cluster may control biosynthesis of a secondary metabolite (small molecule) that is required for or associated with fungal virulence and/or pathogenesis. Similarly, orthologs of the described Cochliobolus gene cluster, e.g., those in Coccidioidus or Candida, may encode gene products that are required for or associated with fungal virulence. As also described hereinbelow, a Cochliobolus iron reductase (SEQ ID NO:49 encoded by SEQ ID NO:48) and a permease and/or MFS transport protein gene (SEQ ID NO:55 encoding SEQ ID NO:56) were identified that are closely linked to a CPS1 peptide synthetase gene, e.g., a DNA molecule comprising SEQ ID NO:2 (GenBank accession no. AF332878) encoding SEQ H)NO:3 (GenBank accession no. AAG53991), which is part of a gene cluster associated with virulence and/or pathogenicity.

Thus, at least one of the genes in the cluster may control biosynthesis of at least one secondary metabolite or other small molecule that is required for or associated with fungal growth, virulence and/or pathogenesis. The fungal produced siderophore may sequester iron from the environment or host to aid in fungal growth. Pseudomonas aeruginosa produces pigments that are likely associated with virulence, e.g., pyocyanin. A derivative of pyrocyanin, pyochelin, is a siderophore that is produced under low iron conditions to sequester iron from the environment for growth of the pathogen. The competition for iron may have a deleterious effect on the host. Similarly, the Cochliobolus iron reductase or permease/transporter or other gene products associated with iron metabolism may compete with the host for Fe and so contribute to the pathogenicity of the fungus. Similarly, orthologs of the described genes in the Cochliobolus gene cluster in other fungi which infect plants or those that infects vertebrate animals may encode gene products that are required for or associated with fungal virulence including iron metabolism genes, e.g., genes associated with secretion of a toxin or siderophore.

Preferably, the nucleic acid segment is obtained or isolatable from a fungal gene which encodes a polypeptide which is substantially similar, and preferably has at least 70%, e.g., 71%, 72%, 73%, 74%, 75%, 76%, 77%, 78%, 79%, 80%, 81%, 82%, 83%, 84%, 85%, 86%, 87%, 88%, 89%, and even 90% or more, e.g., 91%, 92%, 93%, 94%, 95%, 96%, 97%, 98%, up to at least 99%, amino acid sequence identity to, a polypeptide encoded by a nucleic acid sequence comprising any one of SEQ ID NO:46, SEQ ID NO:48, SEQ ID NO:55, or a fragment (portion) thereof which encodes a partial length polypeptide having substantially the same activity of the full length polypeptide. Preferably, the activity of the partial length polypeptide is at least 50%, generally at least 60%, ordinarily at least 70%, preferably at least 80%, more preferably at least 90% and more preferably still at least 95% the activity as the full-length polypeptide. Preferred partial length polypeptides have substantially the same activity as the corresponding full-length polypeptide.

Further provided is an isolated polynucleotide comprising a nucleic acid segment which is substantially similar, and preferably has 70%, e.g., 71%, 72%, 73%, 74%, 75%, 76%, 77%, 78%, 79%, 80%, 81%, 82%, 83%, 84%, 85%, 86%, 87%, 88%, 89%, and even 90% or more, e.g., 91%, 92%, 93%, 94%, 95%, 96%, 97%, 98%, up to at least 99%, nucleotide sequence identity to, a nucleic acid sequence comprising an open reading frame comprising any one of SEQ ID NO: 46, SEQ ID NO:48, or SEQ ID NO:55.

Another aspect of the present invention, as described below, relates to a method for identifying inhibitors of the gene products encoded by the polynucleotides of the invention, which involves contacting the gene product or cell expressing the polynucleotide with agents that are potential inhibitor compounds, and selecting compounds which decrease the activity of the gene product and/or inhibit cell growth. In another embodiment, the invention relates to a method of imparting disease resistance to a plant or other organism by overexpression the CPS1 ortholog of the invention in the plant or other organism.

The nucleic acid molecules of the invention are preferably obtained or isolatable from a gene from fungi that infect vertebrates, including but not limited to mammals, e.g., livestock such as bovine, ovine, porcine, equine and avians such as turkey and chickens and domestic pets including avians, feline and canine, and humans, which genes are related to pathogenesis. For example, preferred nucleic acid molecules of the invention are obtained or isolatable from Ascomycetes (ascomycetes), and the agents of the invention are useful to treat infections due Ascomycota infection, based on the discovery of CPS1, its orthologs and related genes in the cluster, in various ascomycetes human (and plant) pathogens as disclosed herein. Within pathogenic Ascomycetes, the following groups are of interest: Agyriales, Arthoniales, Ascosphaerales, Caliciales, Calosphaeriales, Capnodiales, Chaetothyriales (black yeasts), Cyttariales, Diaporthales, Dothideales, Elaphomycetales, Erysiphales (powdery mildews), Eurotiales (green and blue mold), Gyalectales, Halosphaeriales, Helotiales, Hypocreales, Laboulbeniales, Lecanorales, Lulworthiales, Melanommatales, Meliolales, Microascales, Myriangiales, Neolectales, Onygenales, Ophiostomatales, Ostropales, Patellariales, Pertusariales, Pezizales, Phyllachorales, Pleosporales, Protomycetales, Pyrenulales, Rhytismatales, Saccharomycetes, Schizosaccharomycetales, Sordariales, Taphrinales, Teloschistales, Thelebolaceae, Umbilicariales, Xylariales, anamorphic Ascomycota, unclassified Asconiycota, and Ascomycota incertae sedis.

Regarding Ascomycetes animal pathogens, preferred are pathogenic Onygenales, more particularly the anamorphic Onygenales, which includes coccidioides, and the Onygenaceae and its group Ajellomyces, which includes Histoplasma such as Histoplasma capsulatum, and Blastomycoides such as Blastomycoides dermatitidis. Also preferred are pathogenic Saccharomycetes, more preferably Saccharomycetales, and even more preferably anamorphic Saccharomycetales, which includes Candida species. Also preferred are Chaetothyriales, more preferably Herpotrichiellaceae, even more preferably anamorphic Herpotrichiellaceae, and even more preferably Exophiala, which include the human-pathogenic organisms Exophiala dermatitidis and Exophiala jeanselmei. Also preferred are the Onygenales, more preferably Arthrodermataceae, more preferably anamorphic Arthrodermataceae, and even more preferably Trichophyton, which contain Trichophyton rubrum. Another preferred group is Fungi incertae sedis, more preferably Pneumocystidaceae, and even more preferably Pneuinocystis, which includes the human pathogen Pneumocystis carinii. Yet another preferred group is Eurotiales, more preferred Trichocomaceae, even more preferred anamorphic Trichocoinaceae, and yet even more preferred is Aspergillus species, which contains Aspergillus avenaceus and Aspergillus fumigatis. Another preferred group are those pathogenic fungi in Pleosporales, more preferably Pleosporaceae, yet more preferably anamorphic Pleosporaceae, and even more preferably Altenaria species, which includes airborne Altemaria alternata. Also preferred is Ascomycota incertae sedis, more preferably Mycosphaerellaceae, particularly the anamorphic Mycosphaerellaceae, and more preferably the species Cladosporium, which includes airborne human pathogens. Also preferred are anamorphic Asconiycota, more preferably the species Helminthosporium. Within Onygenales are preferably anamorphic Onygenales, and more preferably the Paracoccidioides species, which includes Paracoccidioides brasiliensis. Also preferred are Microascales, more preferably Microascaceae, and even more preferably Pseudallescheria species, which includes Pseudallescheria boydii. Also preferred are Ophiostomatales, more preferably Ophiostomataceae, yet more preferably anamorphic Ophiostomataceae, and more preferably Sporothrix species, including Sporothrix schenckii.

The term “substantially similar”, when used herein with respect to a polypeptide means a polypeptide corresponding to a reference polypeptide, wherein the polypeptide has substantially the same structure and function as the reference polypeptide, e.g., where the only changes in amino acid sequences are those which do not affect the polypeptide function. When used for a polypeptide or an amino acid sequence, the percentage of identity between the substantially similar and the reference polypeptide or amino acid sequence is at least 70%, e.g., 71%, 72%, 73%, 74%, 75%, 76%, 77%, 78%, 79%, 80%, 81%, 82%, 83%, 84%, 85%, 86%, 87%, 88%, 89%, and even 90% or more, e.g., 91%, 92%, 93%, 94%, 95%, 96%, 97%, 98%, up to at least 99%, wherein the reference polypeptide comprises SEQ ID NO:47, SEQ ID NO:49 or SEQ ID NO:56. One indication that two polypeptides are substantially similar to each other is that an agent, e.g., an antibody, which specifically binds to one of the polypeptides, specifically binds to the other.

In its broadest sense, the term “substantially similar”, when used herein with respect to a nucleotide sequence or nucleic acid segment, means a nucleotide sequence or segment corresponding to a reference nucleotide sequence or nucleic acid segment, wherein the corresponding sequence encodes a polypeptide having substantially the same structure and function as the polypeptide encoded by the reference nucleotide sequence or nucleic acid segment The term “substantially similar” is specifically intended to include nucleotide sequences wherein the sequence has been modified to optimize expression in particular cells. The percentage of identity between the substantially similar nucleotide sequence and the reference nucleotide sequence is at least 70%, e.g., 71%, 72%, 73%, 74%, 75%, 76%, 77%, 78%, 79%, 80%, 81%, 82%, 83%, 84%, 85%, 86%, 87%, 88%, 89%, and even 90% or more, e.g., 91%, 92%, 93%, 94%, 95%, 96%, 97%, 98%, up to at least 99%, preferably wherein the reference sequence comprises SEQ ID NO:46, SEQ ID NO:48, SEQ ID NO:55 or the complement thereof. Sequence comparisons maybe carried out using a Smith-Waterman sequence alignment algorithm (see e.g., Waterman, Introduction to Computational Biology: Maps, sequences and genomes, Chapman & Hall, London (1995) or http://www.htousc.edu/softwarelseqaln/index.html. The local S program, version 1.16, is preferably used with following parameters: mat:1, mismatch penalty: 0.33, open-gap penalty: 2, extended-gap penalty: 2. Further, a nucleotide sequence that is “substantially similar” to a reference nucleotide sequence hybridizes to the reference nucleotide sequence under moderate, stringent, or very stringent, hybridization conditions, e.g., in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

Thus, the invention also includes recombinant nucleic acid molecules which have been modified so as to comprise codons other than those present in the unmodified sequence or have been modified by shuffling. The recombinant nucleic acid molecules of the invention include those in which the modified codons in the unmodified sequence, as well as those that specify different amino acids, i.e., they encode a variant polypeptide having one or more amino acid substitutions relative to the polypeptide encoded by the unmodified sequence.

The invention further includes a nucleotide sequence which is complementary to one (hereinafter “test” sequence) which hybridizes under stringent conditions with the nucleic acid molecules of the invention as well as RNA which is encoded by the nucleic acid molecules of the invention as well as RNA which is encoded by the nucleic acid molecule. When the hybridization is performed under stringent conditions, either the test or nucleic acid molecule of the invention is preferably supported, e.g., on a membrane or DNA chip. Thus, either a denatured test or nucleic acid molecule of the invention is preferably first bound to a support and hybridization is effected for a specified period of time at a temperature of, e.g., between 55 and 70° C., in double strength citrate buffered saline (SC) containing 0.1% SDS followed by rinsing of the support at the same temperature but with a buffer having a reduced SC concentration. Depending upon the degree of stringency required such reduced concentration buffers are typically single strength SC containing 0.1% SDS, half strength SC containing 0.1% SDS and one-tenth strength SC containing 0.1% SDS.

Hence, the isolated nucleic acid molecules of the invention include orthologs of SEQ ID NO:46, SEQ ID NO:48 and SEQ ID NO:55, which includes orthologs of the polypeptides encoded therein. An ortholog is a gene from a different species that encodes a product having the same function as the product encoded by a gene from a reference organism. The encoded ortholog products likely have at least 68 to 70% (substantial) sequence identity to each other. Hence, one embodiment the invention includes an isolated polynucleotide comprising a nucleic acid segment encoding a polypeptide having at least 68 to 70% identity to a polypeptide encoded by SEQ ID NO:46, SEQ ID NO:48 or SEQ ID NO:55. Databases such as GenBank which can be accessed at http://www.ncbi.hlm.hih.gov/, may be employed to identify sequences related to those sequences. Alternatively, recombinant DNA techniques such as hybridization or PCR may be employed to identify sequences related to the sequences. Preferred orthologs include those from dimorphic fungal pathogens including Blastomyces, e.g., B. dermatitidis, Coccidioides, Histoplasma, e.g., H. capsulatum, or Paracoccidiodes, e.g., P. brasiliensis, Loboa, Malassezia, Rhodotorrula, Blastoschizomyces, Trichosporon, Saccharomyces, Ciyptococcus including Cryptococcus neofomans, as well as human pathogens such as Candida albicans, and other pathogenic Candida, e.g., C. tropicalis, C. parapsolosis and C. guiettermondii, Coccidioidus imitis, and Aspergillus fumigatus, Sporothrix schenckii, pathogenic members of the Genera Epidermophyton, Microsporum and Trichophyton, Cladosporium (Xylohypha) trichoides, Cladosporium bantianum, Penicillium marnefii, Exophiala (Wangiella) dermatitidis, Fonsecaea pedrosoi and Dactylaria gallopava (Ochroconis gallopavum), as well as other mycogens.

The invention also provides anti-sense nucleic acid molecules corresponding to the sequences described herein. Also provided are expression cassettes, e.g., recombinant vectors, and host cells, comprising the nucleic acid molecule of the invention in which the nucleic acid segment is in either sense or antisense orientation. Also provided is a microarray, comprising one or more of the nucleic acid molecules of the invention or a portion thereof.

Owing to the dramatically increased incidence of life-threatening opportunistic fungal infections it is now clear that diseases of fungal infection are of major importance. The rise in cases has been particularly apparent in transplant recipients and others who are immunocompromised, especially A/DS patients. Besides more serious infections associated with these vulnerable groups, superficial infections such as ringworm and thrush have also become more prevalent. Despite recognizing the importance of fungi as a cause of disease in man and animals, many of the more serious fungal infections remain difficult to diagnose and treat. Thus, there is a continuing need to identify agents to treat fungal infections of vertebrates, including immunocompromised vertebrates, and complications thereof, e.g., pneumonia, flulike illness, erythema nodosum, erythema marginatum, arthritis, multiple thin-walled chronic cavities, miliary disease, bone and joint infection, skin disease, soft tissue abscesses, meningitis, oropharyngitis, oesophagitis, vaginitis, onychomycosis, endophthalmitis, paronychia, and inflammation of the urinary tract, kidney, lever, brain, gastrointestinal tract, and lung.

Thus, another aspect of the present invention relates to a method for identifying inhibitors of the fungal vertebrate CPS1 ortholog, or fungal iron reductase or permease/MFS transporter of the invention. For example, genes encoding products that are associated with virulence, and agents that bind to or otherwise alter or modulate the activity of that gene product, preferably agents that inactivate or decrease (reduce or inhibit) the activity of the gene product, can be identified. The method comprises contacting the gene product(s) or cells which express the gene product(s) with an agent and then determining or detecting whether the agent binds to, or decreases the activity of, the gene product(s). Such an agent modulates or alters a phenotype of the gene product or cell, e.g., pathogenicity of a cell which expresses the gene product. Modulation or alteration encompasses an increase as well as a decrease in an activity, preferably the modification or alteration in the activity of the gene product or cell having the gene product contacted with the agent is at least 10%, or at least 50%, relative to the activity in an untreated control. In particular, the methods are useful to identify agents that inhibit, reduce or suppress the activity of the polypeptide, e.g., by at least 10%, preferably at least 50%, relative to the activity in an untreated control. Thus, the invention also provides agents identified by the methods of the invention. Preferred agents bind to, more preferably inhibit, the activity of a polypeptide of the invention, e.g., one encoded by a dimorphic fungal pathogen such as one from Blastomyces, Coccidioides, Histoplasma a or Paracoccidiodes, and includes pathogenic Candida, e.g., C. albicans, C. tropicalis, C. parapsolosis and C. guiettermondii. The methods may employ screening agents on wild type fingi and/or recombinant fungi, e.g., fungi which overexpress the polypeptide of interest or do not express that polypeptide, e.g., as a result of expression of antisense sequences or a gene knock out. If the agent is one encoded by DNA, the expression of that DNA in an organism susceptible to the pathogen, e.g., a plant, may provide tolerance or resistance to the organism to the pathogen, preferably by inhibiting or preventing pathogen infection.

Methods of the invention may include stably transforming a susceptible organism of cell with one or more sequences which confer tolerance or resistance operably linked to a promoter capable of driving expression of that nucleotide in the cells of the organism.

Other uses for the nucleic acid molecules or polypeptides of the invention, include the use of the polypeptide to raise either polyclonal antibodies or monoclonal antibodies, e.g., antibodies specific for the polypeptide, to detect antibodies in the serum of a vertebrate, or primers or probes specific for the nucleic acid molecules, which can be employed in diagnostic assays for the presence of the pathogen or for therapeutic purposes, and host cells comprising the nucleic acid molecules, e.g., in antisense orientation, or having a deletion in at least a portion of at least one the genes corresponding to the nucleic acid molecules of the invention. Also, given that the gene may encode a peptide synthetase (Watanabe et al., Chem. Biol., 3, 463 (1996)) the gene product may be useful in therapy, e.g., as an anti-cancer agent, an antibiotic, or as an immunosuppressant.

The agents identified by the methods of the invention may also be subjected to further assays to determine whether the agent is substantially nontoxic to a plant or vertebrate organism to be treated as well as the dose to be administered to the vertebrate organism. For example, for Coccidioides, a murine model may be employed (see, Kirland et al., Infect. Immun., 40: 912 (1983)). This model may also be used for screening for an agent of the invention. Further, the agents identified by the methods of the invention, e.g., those which are non-toxic to a plant or vertebrate to be treated, are useful in methods of preventing or treating a disease or disorder associated with fungal infection, including superficial, subcutaneous or systemic infections. The method comprises administering to a vertebrate or plant in need of such treatment, e.g., a vertebrate that is immunocompromised, an amount of an agent of the invention effective to inhibit or prevent fungal or mycogen infection or growth. For example, humans and non-human animals including livestock and domestic pets may be treated with the agents of the invention, e.g., livestock such as bovine, ovine, porcine, equine and avians such as turkey and chicken and domestic pets including avians, felines and canines. Preferably, the agents are administered topically to a mammal such as a human. Preferred plants include cereals, for example, corn, alfalfa, sunflower, rice, Brassica, canola, soybean, barley, soybean, sugarbeet, cotton, safflower, peanut, sorghum, wheat millet, and tobacco.

Moreover, the agents of the invention may be used in conjunction with other therapeutic agents, e.g., fungicides, mycosides, and vaccines, including amphotericin B and azoles. In addition, the agents may be employed to treat sources of fungal contamination, such as the soil or surface areas or materials on which fungi can survive and/or proliferate. Thus, the agents may be contacted with soil or other surfaces that come in contact with vertebrates. Although this contacting may not eliminate the fungus, it may reduce the risk of airborne dissemination of the fungus or its spores.

Also provided is a computer readable medium having stored thereon a nucleic acid sequence that is substantially similar to any one of SEQ ID NO:46, SEQ ID NO:48, SEQ ID NO:55 or the complement thereof, and a computer system comprising a processor and data storage device wherein said data storage device has stored thereon a nucleic acid sequence that is substantially similar to any one of SEQ ID NO:46, SEQ ID NO:48, SEQ ID NO:55 or the complement thereof. Preferably, the computer system comprises an identifier which identifies features in said sequence. Further provided is a database comprising at least one nucleotide sequence in computer readable form wherein said nucleotide sequence is substantially similar to any one of SEQ ID NO:46, SEQ ID NO:48, SEQ ID NO:55, or the complement thereof. The database, for example, carries out functions comprising determining homology, aligning sequences, adjusting sequence alignments, assembling sequences having overlapping sequence, predicting gene sequence, predicting intron borders, identifying motifs, identifying domains, identifying untranslated regulatory sequences, identifying putative sequencing errors, carries out functional genomics analyses, or carries out shuffling of nucleotide sequences.

The invention also provides a method for generating nucleotide sequences encoding polypeptides having at least one region of homology to SEQ ID NO:46, SEQ ID NO:48, SEQ ID NO:55, or the complement thereof. The method comprises shuffling an unmodified nucleotide sequence which is identical or substantially identical to SEQ ID NO:46, SEQ ID NO:48, SEQ ID NO:55, or the complement thereof. The resulting shuffled nucleotide sequence is expressed and a gene product encoded thereby is selected for altered activity as compared to the activity in a polypeptide encoded by SEQ ID NO:46, SEQ ID NO:48, or SEQ ID NO:55. A DNA molecule comprising a shuffled nucleotide sequence obtainable or produced by the method is also provided. In one embodiment, the shuffled DNA molecule encodes a polypeptide having enhanced tolerance to an inhibitor of the polypeptide encoded by SEQ ID NO:46, SEQ ID NO:48, or SEQ ID NO:55. The shuffled DNA molecule may be operably linked to a promoter to form a chimeric molecule which is introduced to a host cell, e.g., a plant cell.

### BRIEF DESCRIPTION OF THE FIGURES

FIG. 1 provides the structure of amino-acid activating modules identified in peptide synthetase genes (adapted from Stachelhaus and Marahiel, J. Biol. Chem. 270, 6163, 1995; Stachelhaus and Marahiel, FEMS Microbiol. Lett., 125, 3, 1995; Pospiech 1995, supra; Marahiel, 1997, supra). FIG. 1A shows the domain arrangements in two types of modules. Structural variations in the first module (safB1) of the gene safB are also indicated below type I. FIG. 1B shows the correlation between module types and the nature of residues in two fungal peptides. Open box: type I module; filled box: type II module. Each peptide sequence is given below.

FIG. 2 is a restriction map of the cloned sequences surrounding the tagged site. A 11.3 kb genomic region (thick line) was cloned and completely sequenced. The original REMI insertion point in the mutant R.C4.2696 is indicated by a vertical arrow. The asterisks indicate two targeted integration sites in the wild type genome. Two open reading frames (in opposite directions), ORF1 (CPS1, 5.4 kb) and ORF2 (TES1, 1.1 kb) are indicated by open boxes below the map (the positions of putative introns are indicated by vertical bars). Locations of seven overlapping plasmid clones used for sequencing are indicated by thin lines on the top of the map (filled triangles represent the vector sequence in each clone). Sequencing strategy is indicated by arrow above each clone line.

FIGS. 3A-C are schematic representations which show the characterization of modular structure of CPS1. Peptide synthetase and thioesterase are indicated by open boxes; shaded boxes inside indicate functional domains and modules; vertical bars in the shaded boxes indicate highly conserved core sequences. FIG. 3A illustrates the general structure of bacterial and fungal peptide synthetases (adapted from Marahiel, 1997, supra). A peptide synthetase gene cluster is shown on the top. There can be one or more amino acid activating module (cyclosporine synthetase has 11) in each protein; some peptide synthetases have thioesterase domains (TE), which can be either integrated into modules or encoded by a separate gene. Each synthetase can have type L type II or both modules. A type I (minimal) module is enlarged to show organization of core sequences and domains. Some peptide synthetases also have condensation or epimerization domains. FIG. 3B illustrates the organization of saframycin Mx1 synthetase containing 4 amino acid activating modules (Pospiech et al., 1996, supra). SafB1 from the first module is enlarged. Core sequences 1 and 5 in safB1 are weakly conserved (indicated by dashed vertical bars). The remaining domains are typical of type I as shown in FIG. 3A. SafC is a putative O-methyltransferase. FIG. 3C illustrates the organization of CPS1. Sequence analysis revealed two amino acid activating modules (CPS1A and CPSIB), both of which have high similarity to safB1 except that core 2 is weakly conserved. A thioesterase domain is found at the C-terminal region of CPS1B. Three vertical arrows indicate the positions of targeted gene disruptions in the wild type genome that yielded the mutant phenotype. TES1 is a thioesterase encoded by a separate gene (TES1).

FIGS. 4A-C depict DNA gel blots showing DNA-DNA hybridization of ChCPS1 to other fungal genera and species. (A) Cochliobolus species (1−17): C. heterostrophus race T, race O; C. carbonum race 1, race 2; C. victoriae isolates FI3, HvW; C. bicolor, C. dactyloctenii, C. chloridis, C. homomorphus, C. intermedius, C. melinidis, C. melinidis, C. peregianensis, C. perotidis, C. ravenelii and C. sativus. (B) Other Ascomycete genera (1−14): C. carbonum race1 (control), Setosphaeria rostrata, Stemphylium spp., Pyrenophora tritici repentis, Bipolaris sacchari, Alternaria spp., A. solani, Nectria haematococca, Fusarium oxysporum, Glomerella spp. Magnaporthe grisea, F. moniliforme, F. moniliforme (repeat) and A. solani (repeat). (C) Candida albicans compared to C. heterostrophus and closely related species (1-7): C. heterostrophus race T, Bipolaris sacchari, Setosphaeria rostrata, Stemphylium spp., Pyrenophora tritici repentis, Alternaria spp. and Candida albicans (arrowhead). Genomic DNAs were digested with HindIII (A, lanes 1-17; B, lanes 1-11; C, lanes 1-7), XhoI (B, lanes 12 and 14) or BglII (B, lane 13) and probed with the 3.2 kb fragment of CPS1 at high stringency. Weak signals in lanes 3 and 17 (panel A) are due to insufficient DNA loading (confirmed by a repeat experiment).

FIGS. 5A-B show similarity of the cloned CPS1 homologs to C. heterostrophus CPS1. (A) Structural comparison of the four CPS1 homologs to ChCPS1 (As=Alternaria solani; Pt=Pyrenophora teres; Fg=Fusarium graminearium; Ci=Coccidioides imitus). ORFs are indicated by the open boxes; shaded boxes inside indicate functional domains; vertical bars indicate conserved motif sequences found in nonribosomal peptide synthetases (NRPS) as defined by Stachelhaus and Marahiel (Stachelhaus and Marahiel, 1995, supra; Marahiel, 1997, supra) (dashed bars indicate weak conservation). The black bulbs indicate the position of putative introns. Cores 1-5: adenylation; core 6: thiolation; TE: thioesterase. The distance between core sequences is not drawn in exact scale. The name of proteins is on the left of ORF box and the number of amino acids on the right. The unidentified regions of AsCPS1, PtCPS1 and CiCPS1 are indicated by dash-lined boxes. The similarity to ChCPS1 (in the overlapping region only) is given in the parentheses under the protein names in the order: nucleotide identity/amino acid identity/amino acid similarity. The positions of the ChCPS1 amino acids 220 and 1040(corresponding to the first and the last amino acid of CiCPS1) are indicated by open arrows; the positions 511 and 1269 (to the first and the last amino acids of AsCPS1 and PtCPS1) are indicated by filled triangles. (B) Amino acid alignment of the four CPS1 homologs to ChCPS1. 530 amino acids aligned to the amino acids 511-1040 of ChCPS1 (SEQ ID NO:186) are shown (SEQ ID NOs: 51-54). The identical residues are in uppercase and the similar residues in lowercase. Consensus of sequences similar to the typical NRPS signature motifs is underlined. The putative cyclization domain motif “D XXXXD/EXXS/A” (SEQ ID NO:60) is underlined.

FIG. 6 shows the results of a BLAST search using FgCPS1 (SEQ ID NO:41) as the query sequence.

FIG. 7A shows the results of a BLAST search using CiCPS1 (SEQ ID NO:47) as the query sequence.

FIG. 7B shows an alignment of amino acid sequence of FgCPS1 (SEQ ID NO:41), AsCPS1 (SEQ ID NO:43), PtCPS1 (SEQ ID NO:45), CiCPS1 (SEQ ID NO:47), and ChCPS1 (SEQ ID NO:3).

FIGS. 8A-C show the sequencing strategy (A), restriction map (B), genome organization (C) for the ChCPS1 gene cluster. SEQ ID NO:59 represents the sequence of genes clustered near ChCPS1. SEQ ID NO:187 and 188 represent the DNA corresponding to and amino acid sequence encoded by ORF 16, respectively. SEQ ID NO:189 and 190 represent the DNA corresponding to and amino acid sequence corresponding to ORF 10, respectively. SEQ ID NO:191 and 192 represent the DNA corresponding to and amino acid sequence encoded by ORF 11, respectively. SEQ ID NO:193 and 194 represent the DNA corresponding to and amino acid sequence encoded by ORF 12, respectively. SEQ ID NO:195 and 196 represent the DNA corresponding to and amino acid sequence encoded by ORF 13, respectively. SEQ ID NO:197 and 198 represent the DNA corresponding to and amino acid sequence encoded by ORF 14, respectively. SEQ ID NO:199 and 200 represent the DNA corresponding to and amino acid sequence encoded by ORF 3, respectively. SEQ ID NO:201 and 202 represent the DNA corresponding to and amino acid sequence encoded by ORF 5, respectively. SEQ ID NO:203 and 204 represent the DNA corresponding to and amino acid sequence encoded by ORF 6, respectively. SEQ ID NO:205 and 206 represent the DNA corresponding to and amino acid sequence encoded by ORF 7, respectively. SEQ ID NO:207 and 208 represent the DNA corresponding to and amino acid sequence encoded by ORF 8, respectively. SEQ ID NO:209 and 210 represent the DNA corresponding to and amino acid sequence encoded by ORF 9, respectively.

FIG. 9A shows the results of a BLAST search using SEQ ID NO:49 (an iron reductase encoded by SEQ ID NO:48) as the query sequence.

FIG. 9B shows an alignment of amino acid sequence of a Cochliobolus iron reductase (SEQ ID NO:49) and a S. cerevisiae reductase (SEQ ID NO:184).

FIG. 9C illustrates a DNA comprising SEQ ID NO:48 (SEQ ID NO:211).

FIG. 9D illustrates the amino acid sequence (SEQ ID NO:212) encoded by SEQ ID NO:211.

FIG. 10 shows the results of a BLAST search using the polypeptide (SEQ ID NO:56) encoded by SEQ ID NO:55 (a Cochliobolus permease and/or MFS transporter) as the query sequence.

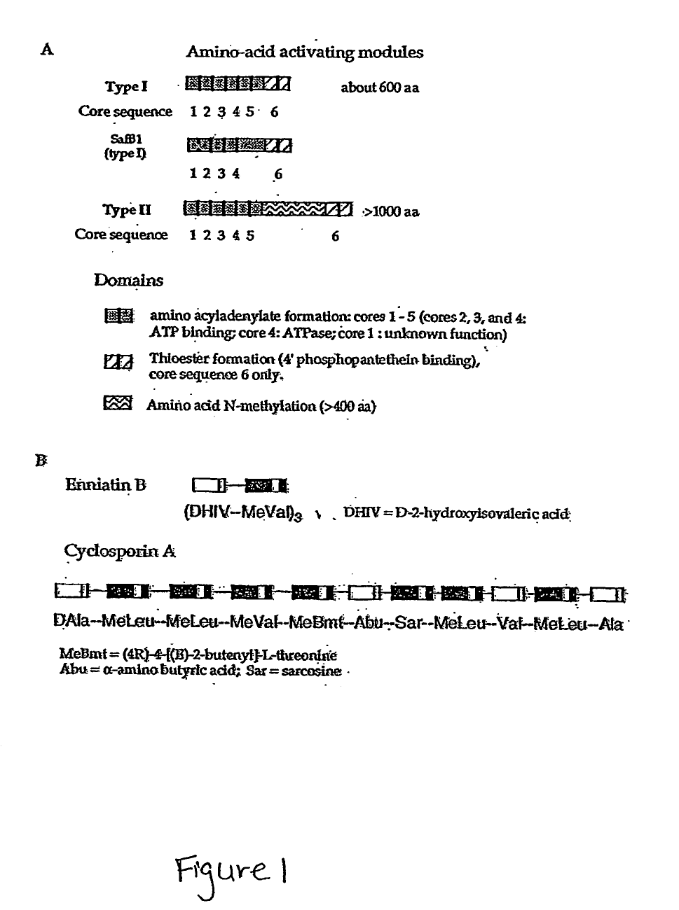

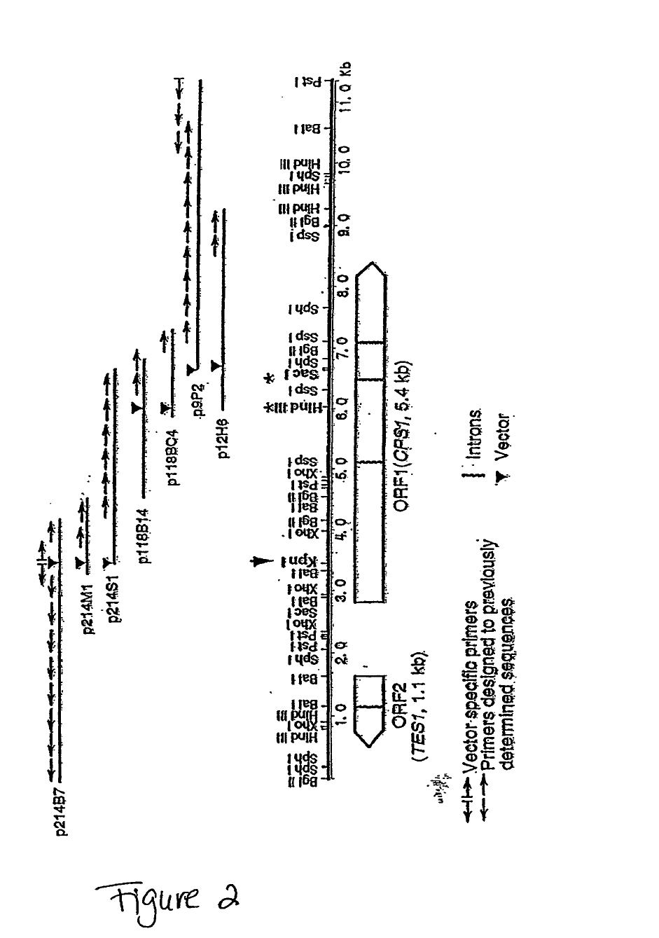

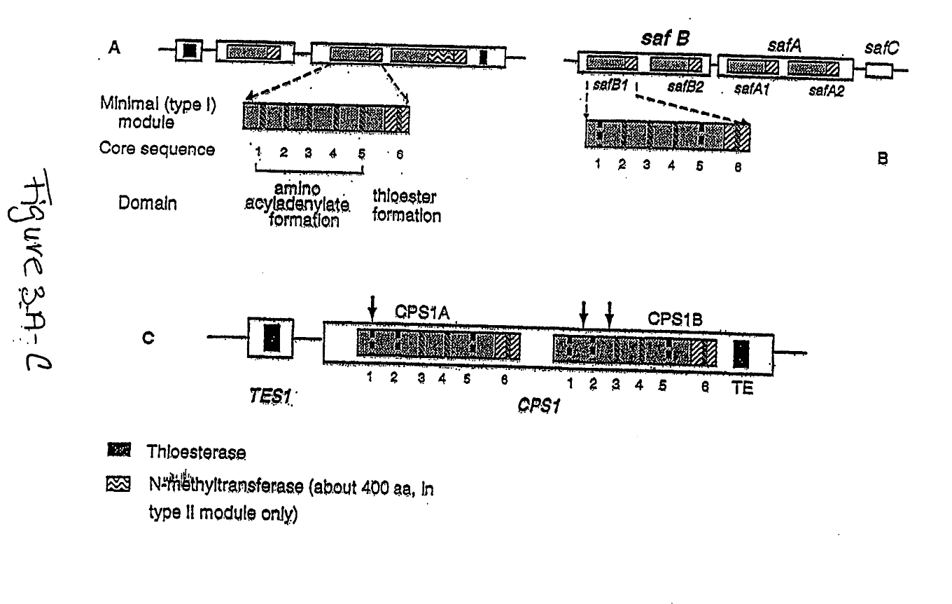

### DETAILED DESCRIPTION OF THE INVENTION

Definitions

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucl. Acids Res., 19:508 (1991); Ohtsuka et al., JBC, 260:2605 (1985); Rossolini et al., Mol. Cell. Probes, 8:91 (1994). Although nucleotides are usually joined by phosphodiester linkages, polymeric nucleotides joined by peptide linkages (peptide nucleic acids) are also included (Neilsen and Egholm, Peptide Nucleotide Acids: Protocols and Applications, Horizon Scientific Press, Wymondham, Norfolk UK, 1999). A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers. The terms “nucleic acid”, “nucleic acid molecule”, “nucleic acid fragment” or “nucleic acid sequence or segment” may also be used interchangeably with gene, cDNA, DNA and RNA encoded by a gene.

The invention encompasses isolated or substantially purified nucleic acid or protein compositions. In the context of the present invention, an “isolated” or “purified” DNA molecule or an “isolated” or “purified” polypeptide is a DNA molecule or polypeptide that, by the hand of man, exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention.

By “fragment” or “portion” is meant a full length or less than full length of the nucleic acid sequence encoding, or the amino acid sequence of, a polypeptide or protein. Alternatively, fragments or portions of a nucleotide sequence that are useful as hybridization probes generally do not encode fragment proteins retaining biological activity. Thus, fragments or portions of a nucleotide sequence may range from at least about 6 nucleotides, about 9, about 12 nucleotides, about 20 nucleotides, about 50 nucleotides, about 100 nucleotides or more. By “portion” or “fragment”, as it relates to a nucleic acid molecule, sequence or segment of the invention, when it is linked to other sequences for expression, is meant a sequence having at least 80 nucleotides, more preferably at least 150 nucleotides, and still more preferably at least 400 nucleotides. If not employed for expressing, a “portion” or “fragment” means at least 6, about 9, preferably 12, more preferably 15, even more preferably at least 20, consecutive nucleotides, e.g., probes and primers (oligonucleotides), corresponding to the nucleotide sequence of the nucleic acid molecules of the invention.

By “resistant” is meant an organism, e.g., a plant or animal, that exhibits substantially no phenotypic changes as a consequence of infection with a pathogen By “tolerant” is meant an organism which, although it may exhibit some phenotypic changes as a consequence of infection, does not have a decreased reproductive capacity or substantially altered metabolism.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

“Naturally occurring” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

A “marker gene” encodes a selectable or screenable trait.

“Selectable marker” is a gene whose expression in a cell gives the cell a selective advantage. The selective advantage possessed by the cells transformed with the selectable marker gene may be due to their ability to grow in the presence of a negative selective agent, such as an antibiotic or a herbicide, compared to the growth of non-transformed cells. The selective advantage possessed by the transformed cells, compared to non-transformed cells, may also be due to their enhanced or novel capacity to utilize an added compound as a nutrient, growth factor or energy source. Selectable marker gene also refers to a gene or a combination of genes whose expression in a cell gives the cell both a negative and/or a positive selective advantage.

The term “chimeric” refers to any gene or DNA that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

A “transgene” refers to a gene that has been introduced into the genome by transformation and is stably maintained. Transgenes may include, for example, DNA that is either heterologous or homologous to the DNA of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but that is introduced by gene transfer.

The terms “protein,” “peptide” and “polypeptide” are used interchangeably herein.

By “variants” is intended substantially similar sequences. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis which encode the native protein, as well as those that encode a polypeptide having amino acid substitutions. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“DNA shuffling” is a method to introduce mutations or rearrangements, preferably randomly, in a DNA molecule or to generate exchanges of DNA sequences between two or more DNA molecules, preferably randomly. The DNA molecule resulting from DNA shuffling is a shuffled DNA molecule that is a non-naturally occurring DNA molecule derived from at least one template DNA molecule. The shuffled DNA preferably encodes a variant polypeptide modified with respect to the polypeptide encoded by the template DNA, and may have an altered biological activity with respect to the polypeptide encoded by the template DNA.

The nucleic acid molecules of the invention can be optimized for enhanced expression in an organism of interest (Wada et al., Nucl Acids Res. 18:2367 (1990). For plants see, for example, EPA035472; WO91/16432; Perlak et al., Proc. Natl. Acad. Sci. USA, 88:3324 (1991); and Murray et al., Nucl Acids Res. 17:477 (1989). In this manner, the genes or gene fragments can be synthesized utilizing plant-preferred codons. See, for example, Campbell and Gowri, 1990 for a discussion of host-preferred codon usage. Thus, the nucleotide sequences can be optimized for expression in any plant. It is recognized that all or any part of the gene sequence may be optimized or synthetic. That is, synthetic or partially optimized sequences may also be used. Variant nucleotide sequences and proteins also encompass sequences and protein derived from a mutagenic and recombinogenic procedure such as DNA shuffling. With such a procedure, one or more different coding sequences can be manipulated to create a new polypeptide possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, Nature, 370:389 (1994); Crameri et al., Nature Biotech., 15:436 (1997); Moore et al., JMB 272:336 (1997); Zhang et al., Proc. Natl. Acad. Sci. USA, 94:4504 (1997); Crameri et al., Nature, 391:288 (1998); and U.S. Pat. Nos. 5,605,793 and 5,837,458.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Recombinant DNA molecule” is a combination of DNA sequences that are joined together using recombinant DNA technology and procedures used to join together DNA sequences as described, for example, in Sambrook et al., Cold Spring Harbor, N.Y.: Cold Spring Harbor Laboratory Press (1989).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “microarray” as used herein is a solid support and a plurality of different oligonucleotides attached to the support. Each of the different oligonucleotides is attached to the surface of the solid support in a different defined region, has a different determinable sequence, and is at least six nucleotides in length. Preferably, at least one of the different oligonucleotides is derived from a region of a polynucleotide having a nucleotide sequence selected from SEQ ID NO:46, SEQ ID NO:48 and SEQ ID NO:55, or the complement thereof.

A “homologous” DNA sequence is a DNA sequence that is naturally associated with a host cell into which it is introduced.

“Wild-type” refers to the normal gene, e.g., a gene found in the highest frequency in a particular population, or organism found in nature without any known mutation.

“Genome” refers to the complete genetic material of an organism.

“Vector” is defined to include, inter alia, any plasmid, cosmid, phage or binary vector in double or single stranded linear or circular form which may or may not be self transmissible or mobilizable, and which can transform prokaryotic or eukaryotic host either by integration into the cellular genome or exist extrachromosomally (e.g., autonomous replicating plasmid with an origin of replication).

Specifically included are shuttle vectors by which is meant a DNA vehicle capable, naturally or by design, of replication in two different host organisms, which may be selected from actinomycetes and related species, bacteria and eukaryotic (e.g., higher plant, mammalian, yeast or fungal cells).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

A transcriptional cassette will include in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source. For expression in plants, convenient termination regions are available from the Ti-plasmid of A. tumefaciens, such as the octopine synthase and nopaline synthase termination regions. See also, Guerineau et al., Mol. Gen. Genetics, 262:141 (1991); Proudfoot, Cell, 64:671 (1991); Sanfacon et al., Genes Dev., 5:141 (1991); Mogen et al., Plant Cell 2:1261 (1990); Munroe et al., Gene, 91:151 (1990); Ballas et al., Nucl. Acids Res., 17:7891 (1989); Joshi et al., Nucl. Acids Res., 15:9827 (1987).

An oligonucleotide corresponding to a nucleic acid molecule of the invention maybe about 30 or fewer nucleotides in length (e.g., 9, 12, 15, 18, 20, 21 or 24, or any number between 9 and 30). Generally specific primers are upwards of 14 nucleotides in length. For optimum specificity and cost effectiveness, primers of 16-24 nucleotides in length maybe preferred. Those skilled in the art are well versed in the design of primers for use processes such as PCR. If required, probing can be done with entire restriction fragments of the gene disclosed herein which may be 100's or even 1000's of nucleotides in length.

“Coding sequence” refers to a DNA or RNA sequence that codes for a specific amino acid sequence and excludes the non-coding sequences 5′ and 3′ to the coding sequence. It may constitute an “uninterrupted coding sequence”, i.e., lacking an intron, such as in a cDNA or it may include one or more introns bounded by appropriate splice junctions, e.g., as may be found in genomic DNA. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (“codon”) in a coding sequence that specifies initiation and chain termination, respectively, of protein synthesis (mRNA translation).

A “functional RNA” refers to an antisense RNA, ribozyme, or other RNA that is not translated.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences which may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., Mol. Biotech., 3:225 (1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., Plant Cell, 1, 671, 1989.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors which control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene or a transgene in plants. For example, in the case of antisense constructs, expression may refer to the transcription of the antisense DNA only. In addition, expression refers to the transcription and stable accumulation of sense (mRNA) or functional RNA. Expression may also refer to the production of protein.

“Altered levels” refers to the level of expression in transgenic cells or organisms that differs from that of normal or untransformed cells or organisms.

“Overexpression” refers to the level of expression in transgenic cells or organisms that exceeds levels of expression in normal or untransformed cells or organisms.

“Antisense inhibition” refers to the production of antisense RNA transcripts capable of suppressing the expression of protein from an endogenous gene or a transgene. “Co-suppression” and “transwitch” each refer to the production of sense RNA transcripts capable of suppressing the expression of identical or substantially similar transgene or endogenous genes (U.S. Pat. No. 5,231,020).

“Gene silencing” refers to homology-dependent suppression of viral genes, transgenes, or endogenous nuclear genes. Gene silencing may be transcriptional, when the suppression is due to decreased transcription of the affected genes, or post-transcriptional, when the suppression is due to increased turnover (degradation) of RNA species homologous to the affected genes (English et al., Plant Cell, 8:179 (1996). Gene silencing includes virus-induced gene silencing (Ruiz et al., Plant Cell, 10:937 (1998).

“Chromosomally-integrated” refers to the integration of a foreign gene or DNA construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller, CABIOS, 4:11 (1988); the local homology algorithm of Smith et al., Adv. Appl. Math., 2:482 (1981); the homology alignment algorithm of Needleman and Wunsch, JMB, 48:443 (1970); the search-for-similarity-method of Pearson and Lipman, Proc. Natl. Acad. Sci. USA 85:2444 (1988); the algorithm of Karlin and Altschul, Proc. Natl. Acad. Sci. USA, 87:2264 (1990), modified as in Karlin and Altschul, Proc. Natl. Acad. Sci. USA, 90:5873 (1993).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al., Gene, 73:237 (1988); Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol. 24:307 (1994). The ALIGN program is based on the algorithm of Myers and Miller, supra. The BLAST programs of Altschul et al., JMB, 215:403 (1990); Nucl. Acids Res., 25:3389 (1990), are based on the algorithm of Karlin and Altschul supra.

Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences (see, e.g., Karlin & Altschul (1993), supra). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al., 1997. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix (see Henikoff & Henikoff, 1989). See http://www.ncbi.nlm.nih.gov. Alignment may also be performed manually by inspection.

For purposes of the present invention, comparison of nucleotide sequences for determination of percent sequence identity to the sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, more preferably at least 80%, 90%, and most preferably at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, preferably 80%, 81%, 82%, 83%, 84%, 85%, 86%, 87%, 88%, or 89%, more preferably at least 90%, 91%, 92%, 93%, or 94%, or even more preferably, 95%, 96%, 97%, 98% or 99%, sequence identity to the reference sequence over a specified comparison window. Preferably, optimal alignment is conducted using the homology alignment algorithm of Needleman and Wunsch, 1970, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, 1984; Tₘ 81.5° C.+16.6 (log M)+0.41 (% GC)−0.61 (%-form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, 1993. Generally, highly stringent hybridization and wash conditions are selected to be about 5° C. lower than the thermal melting point (Tₘ) for the specific sequence at a defined ionic strength and pH.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1×to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5×to 1×SSC at 55 to 60° C.

The following are examples of sets of hybridization/wash conditions that may be used to clone orthologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides of the invention may be altered in various ways including amino acid substitutions, deletions, tuncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82:488 (1985); Kunkel et al., Meth. Enzymol., 154:367 (1987); U.S. Pat. No. 4,873,192; Walker and Gaastra, Techniques in Mol. Biol. (MacMillan Publishing Co. (1983), and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass both naturally occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (1); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). See also, Creighton, 1984. In addition, individual substitutions, deletions or additions which alter, add or delete a single amino acid or a small percentage of amino acids in an encoded sequence are also “conservatively modified variations.”

“Germline cells” refer to cells that are destined to be gametes and whose genetic material is heritable.

The word “plant” refers to any plant, particularly to seed plant, and “plant cell” is a structural and physiological unit of the plant, which comprises a cell wall but may also refer to a protoplast. The plant cell may be in form of an isolated single cell or a cultured cell, or as a part of higher organized unit such as, for example, a plant tissue, or a plant organ.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

The term “altered plant trait” means any phenotypic or genotypic change in a transgenic plant relative to the wild-type or non-transgenic plant host.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”. Examples of methods of transformation of plants and plant cells include Agrobacterium-mediated transformation (De Blaere et al., Meth. Enzymol., 143:277 (1987) and particle bombardment technology (Klein et al., Nature, 327:70 (1987); U.S. Pat. No. 4,945,050). Whole plants may be regenerated from transgenic cells by methods well known to the skilled artisan (see, for example, Fromm et al., Biotech., 8:833 (1990).

“Transformed,” “transgenic,” and “recombinant” refer to a host cell or organism such as a bacterium or a plant into which a heterologous nucleic acid molecule has been introduced. The nucleic acid molecule can be stably integrated into the genome generally known in the art and are disclosed in Sambrook et al., 1989, supra. See also Innis et al., PCR Protocols, Academic Press (1995); and Gelfand, PCR Strategies, Academic Press (1995); and Innis and Gelfand, PCR Methods Manual, Academic Press (1999). Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” plants or calli have been through the transformation process and contain a foreign gene integrated into their chromosome. The term “untransformed” refers to normal plants that have not been through the transformation process.

A “transgenic” organism is an organism having one or more cells that contain an expression vector.

“Transiently transformed” refers to cells in which transgenes and foreign DNA have been introduced but not selected for stable maintenance.

“Stably transformed” refers to cells that have been selected and regenerated on a selection media following transformation.

“Genetically stable” and “heritable” refer to chromosomally-integrated genetic elements that are stably maintained in the plant and stably inherited by progeny through successive generations.

“Enzyme activity” means herein the ability of an enzyme to catalyze the conversion of a substrate into a product. A substrate for the enzyme comprises the natural substrate of the enzyme but also comprises analogues of the natural substrate which can also be converted by the enzyme into a product or into an analogue of a product. The activity of the enzyme is measured for example by determining the amount of product in the reaction after a certain period of time, or by determining the amount of product in the reaction after a certain period of time, or by determining the amount of substrate remaining in the reaction mixture after a certain period of time. The activity of the enzyme is also measured by determining the amount of an unused co-factor of the reaction remaining in the reaction mixture after a certain period of time or by determining the amount of used co-factor in the reaction mixture after a certain period of time. The activity of the enzyme is also measured by determining the amount of a donor of free energy or energy-rich molecule (e.g., ATP, phosphoenolpyruvate, acetyl phosphate or phosphocreatine) remaining in the reaction mixture after a certain period of time or by determining the amount of a used donor of a free energy or energy-rich molecule (e.g., ADP, pyruvate, acetate or creatine) in the reaction mixture after a certain period of time.

“Fungicide” is a chemical substance used to kill or suppress the growth of fungal cells.

An “inhibitor” is a chemical substance that causes abnormal growth, e.g., by inactivating the enzymatic activity of a protein such as biosynthetic enzyme, receptor, signal transduction protein, structural gene product, or transport protein that is essential to the growth or survival, or alters the virulence or pathogenicity, of the fungus. In the context of the instant invention, an inhibitor is a chemical substance that alters the activity encoded by any one of SEQ ID NO:47, SEQ ID NO:49, SEQ ID NO:56 or their orthologs.

“Isogenic” fungi are genetically identical, except that they may differ by the presence or absence of a heterologous DNA sequence.

A “substrate” is the molecule that an enzyme naturally recognizes and converts to a product in the biochemical pathway in which the enzyme naturally carries out its function, or is a modified version of the molecule, which is also recognized by the enzyme and is converted by the enzyme to a product in an enzymatic reaction similar to the naturally-occurring reaction.

“Tolerance” as used herein is the ability of an organism, e.g., a fungus, to continue essentially normal growth or function when exposed to an inhibitor or fungicide in an amount sufficient to suppress the normal growth or function of native, unmodified fungi.

The Nucleic Acid Molecules of the Invention and Uses Thereof

The involvement of peptide synthetase genes in fungal pathogenesis to plants has been genetically tested only in two previous studies. In C. carbonum, disruption of both copies of the HTS1 gene, which encodes HC-toxin synthetase, caused loss of ability to make HC-toxin and the fungus became nonpathogenic on HC-toxin sensitive corn plants (Panaccione et al, PNAS, 89, 6590, 1992), indicating that the HC-toxin synthetase gene is a pathogenicity determinant. In Fusarium avenaceum, the enniatin-nonproducing transformants were obtained by disruption of enniatin synthetase encoding gene (esyn1) and these transformants displayed significantly reduced virulence in a potato tuber tissue assay (Herrmann et al., 1996) indicating that enniatin synthetase gene is a virulence factor in pathogenesis by the fungus. In these two pathosystems, only one fungal secondary metabolite (the peptide toxin) was studied. In contrast, the polyketide T-toxin has been well studied in C. heterostrophs and has been confirmed to be a host-specific virulence factor (Yoder and Turgeon, 1996; Yoder et al., 1997, supra) and this study demonstrated that a second secondary metabolite, the hypothetical CPS1 toxin is also involved in pathogenesis by the fungus. Unlike the T-toxin biosynthetic genes such as PKS1 and DEC1 that are found only in race T (Yang et al., 1996, supra; Rose et al., 1996, supra), CPS1 is found in both race O and race T. Disruption of CPS1 in either race causes dramatically reduced fungal virulence as tested on N-cytoplasm corn. This result suggests that CPS1 toxin could be the same as the “race O” toxin proposed previously (Yoder, 1981). However, as disclosed herein, CPS1 is a CoA ligase.

Interestingly, a Tox⁺, cps1⁻ mutant also show reduced virulence on T-cytoplasm corn although it produced the same amount of T-toxin as wild type race T. This is unusual because the interaction between T-toxin and the T-corn-unique URF13 protein is highly specific; the same outcomes should be expected if two strains that produce the same amount of T-toxin attack the same host, T-corn. The most likely explanation for this result is that the fungal growth in planta has been inhibited by the host plant and the poor growth results in reduced T-toxin production which is normal when the fungus is grown in culture. Reduced virulence on T-cytoplasm corn is due to the reduced T-toxin production as that seen in leaky Tox⁻ mutants. This inhibition of growth could be due to the failure of suppression of the host defense mechanism by the fungus, which is mediated by the CPS1 controlled peptide toxin. A cps1⁻ mutant that fails to produce this “suppresser” could not be able to colonize plant tissues as vigorously as wild type does, resulting in the reduced ability to cause disease as indicated by the smaller lesion phenotype. If this turns out to be the case, CPS1 should be considered as a general virulence factor as proposed for enniatin.

It is possible that cps1⁻ mutants are still be able to produce a certain amount of CPS1 toxin. One probability is the gene has not been completely activated by insertional mutagenesis or targeted disruption. The original REMI insertion occurred at core sequence 1 of CPS1A, a region that might be not critical (function of core 1 is unknown). The second targeted site is located between cores 1 and 2 of CPS1B and the third is located between cores 2 and 3 of the same module. All three insertions do not disrupt critical motifs. On the other hand, CPS1 contains a number of in-frame start codons and some of them are located immediately downstream of these insertion sites. It is possible that each of these disruptions actually resulted in two subtranscripts, one is transcribed normally from the start codon of CPS1 and stops at the insertion site and second is transcribed near one of these in-frame ATGs downstream of the insertion site and stops at the end of CPS1. Both transcripts could give a truncated protein that still has enzymatic activities. But these separate enzymes might have affinities for their substrates lower than that of holoenzyme. The reduced production of CPS1 toxin might be due to the CPS1 holoenzyme having been split into two fractions by the vector insertion and the resulting truncated proteins being much less active than the original polypeptide. This hypothesis can be tested by construction a C. heterostrophus strain in which the entire CPS1 encoding sequence has been deleted.

The second possibility is the existence of multiple copies of CPS1 in the genome. Previous studies have demonstrated that the gene encoding HC-toxin synthetase (HTS1) is duplicated in the genome and both copies (HTS1-1 and HTS1-2) are 270 kb apart in most Tox2+isolates of C. carbonum (Ahn and Walton, Plant Cell, 8, 887, 1996). Disruption of either copy reduced HTS1 activity but did not affect HC-toxin production; when both copies were disrupted, HC-toxin production was abolished (Panaccione et al, 1992, supra). But in contrast to the case of HTS1, gel blot analysis does not indicate the presence of a second copy of CPS1 and disruption of CPS1 does affect the production of the putative toxin. It is unlikely that two genes with similar organization are in the genome. An alternative postulation is that there may be a second gene which encodes a protein with the same enzyme activity as CPS1 but does not have significant sequence homology to CPS1. This hypothesis is hard to test unless this gene is clustered with CPS1 and can be recovered by chromosome walking.

Pathogenesis by C. heterostrophus to corn involves at least two secondary metabolites: the T-toxin, a host specific factor which determines high virulence on a particular host, T-com and the hypothetical CPS1 toxin, a general factor (either virulence or pathogenicity factor) which contributes to basic mechanisms underlying the disease establishment by the fungus in common host plants.

By genomic DNA hybridization, C. heterostrophus CPS1 homologs were found in 16 additional fungal species belonging to 5 genera. Hybridization signals for some were as strong as the C. heterostrophus gene, indicating that CPS1 is highly conserved among these fungi. This conservation appears to match the taxonomic relationships between these species. Cochliobolus (anamorph Bipolaris) and Setosphaeria (anamorph Exserohilum) are closely related genera.

Two species, C. victoriae and C. carbonum, which are able to cross to each other and thus may not be different species (Scheffer et al., 1967; Yoder et al., 1989), showed the same hybridization pattern to CPS1. B. sacchari, the closest asexual relative of C. heterostrophus, hybridized to two HindIII fragments that were only seen in C. heterostrophus itself, but all other species gave only one distinct polymorphic band. Phylogenetic analyses using the internal transcribed spacer (ITS) sequences and fragments of the GPD (vanWert and Yoder, 1992) and MAT genes (Turgeon et al., Mol. Gen. Genet., 238, 270, 1993) also put C. victoriae/C. carbonum and C. heterostrophus/B. sacchari closest to each other (Turgeon and Berbee, 1997). These results might imply that CPS1 has coevolved with these genes.

The genera Cochliobolus and Setosphaeria include many plant pathogenic species that are commonly associated with leaf spots or blights, mainly on cultivated cereals and wild grasses (Sivanesan, 1987; Alcorn, 1988). This group of phytopathogenic fungi includes both mild pathogens and severe pathogens that often produce host-specific toxins (Yoder, 1980, supra). One of the essential questions is whether or not the various diseases on diverse host plants caused by these fungi involve common factors or depend only on individual specific factors, such as host-specific toxins.

Previous studies have shown that host-specific toxins can be critical factors for determining either virulence or host-range, but they do not account for general pathogenicity since they are produced only by certain isolates in the species and the corresponding biosynthetic genes are found only in these toxin-producing isolates (Yoder et al., 1997, supra). In contrast, CPS1 homologs are found in all Cochliobolus and Setosphaeria species tested so far, suggesting they are a common factor shared by this group. Disruption of the CPS1 homolog in the oat pathogen C. victoriae caused dramatically reduced virulence to victorin-susceptible oats although the transformants produced wild type levels of victorin. This result is similar to that with C. heterostrophus race T, in which cps1⁻ disruptants still produced wild type levels of T-toxin but showed reduced virulence on T-cytoplasm corn. These results argue strongly that host-specific toxins alone are not sufficient in determining the ultimate outcome of fungus/plant interactions and suggest that the establishment of disease by these fungi also requires CPS1, which might control a pathway for general pathogenicity.

In the early 1990s, studies on pathogenesis by uropathogenic E. coli led to the identification of pathogenicity gene clusters, termed “pathogenicity islands” (Hecker et al., 1990; Blum et al., 1994). Subsequently, similar gene clusters were identified in additional animal or human bacterial pathogens, including Yersinia pestis, Helicobacter pylon and Salmonella typhimuriun. These islands often contain genes for production of toxins or genes encoding proteins that are capable of interacting with host defense factors or required for type III secretion systems that deliver virulence proteins into host cells. Usually, they are found only in pathogenic strains (or species); in rare cases, they occur in nonpathogenic strains of the same species or related species (Hacker et al., Mol. Microbiol., 23, 1089, 1997).

In phytopathogenic bacteria, hrp gene clusters have been referred to as “pathogenicity islands” because they have several features in common with “pathogenicity islands” in animal pathogenic bacteria, i.e., they are found only in pathogenic species (required for plant pathogenicity) and contain highly conserved genes (hrc genes) defining the type III protein secretion system (Alfano and Collmer, 1996; Barinaga, 1996).

In plant pathogenic fungi, genes or gene clusters with characteristics of “pathogenicity islands” have been identified from certain species, i.e., in Nectria haematococca, the PDA genes for detoxifying the pea phytoalexin and other pea pathogenicity genes (PEP) are located on dispensable chromosomes that are found in all isolates pathogenic to pea but usually absent in all nonpathogenic isolates (VanEtten et al., Antonie Van Leeuwenhoek, 65, 263, 1994; Liu et al., 1997, supra). In the genus Cochliobolus, the Tox2 gene cluster controlling the biosynthesis of HC-toxin is found only in C. carbonum race 1 (pathogenic to hm1hm1 corn) and the Tox1 genes controlling T-toxin production are found only in C. heterostrophus race T (highly virulent on T-cytoplasm corn); all other races of the same species and all other fungal species tested so far lack these Tox genes (Ahn and Walton, 1996, supra; Yang et al., 1996, supra; Yoder et al., 1997, supra).

CPS1 differs in two important ways compared to these fungal “pathogenicity islands”. First, it is highly conserved among several phytopathogenic Cochliobolus species and relatives. Second, like certain bacterial “pathogenicity islands”, CPS1 also has homologs in “nonpathogenic” species. C. homomorphus and C. dactyloctenii, neither of which causes disease on plants, hybridized strongly to CPS1. This may reflect genetic changes in the “pathogenicity island” that resulted in loss of pathogenicity. In the bacterial genus Listeria, which includes several human or animal pathogenic species harboring highly conserved “pathogenicity islands”, the “pathogenicity island” homolog in the nonpathogenic species (L. seeligeri) was found to be “silent” due to a mutation that occurred in the promoter region of a critical regulatory gene in the cluster (Hacker et al., 1997, supra). These features suggest that the CPS1 gene cluster and homologs could define a new group of fungal “pathogenicity islands”.

It is known that the evolution of pathogenicity involves two major processes. A pathogenic microorganism could originate from nonpathogenic progenitors by slow modifications (such as point mutations and genetic recombination) of genes that were adapted for parasitic growth on hosts or by the integration of large fragments of “alien” DNA into the genome that enable the recipient to attack particular hosts (gene horizontal transfer). The latter can occur in the recent or distant evolutionary past. Subsequent vertical transmission in the lineage (if the transferred gene is stable in the recipient genome) would result in the preserve of the gene in all species that diverged after the acquisition of the gene(s) (Scheffer, 1991; Arber, Gene, 135, 49, 1993; Krishnapillai, 1996; Burdon and Silk, 1997).

In the past few years, substantial evidence has become available that supports the hypothesis of gene horizontal transfer. All “pathogenicity islands” in animal pathogenic bacteria are believed to have been acquired by a horizontal transfer event (recent or past) because they usually differ in G+C content from the recipient genome and have transposable elements at the boundaries of the gene clusters (Hacker et al., 1997, supra). The hrp “pathogenicity islands” do not show a significant difference in G+C content or association with transposable elements, but they are also believed to have arisen similarly because hrc genes in these “pathogenicity islands” show high similarity to genes defining the type III protein secretion system found in animal pathogenic bacteria as mentioned above (Alfano and Collmer, 1996; and Barinaga, 1996).

Although CPS1 itself has several typical fungal introns and a G+C content (51.5%) similar to most known fungal genes, genomic regions (about 1.5 kb) flanking the gene have higher G+C content (>60%). Several short G+C-rich regions are also found in the gene cluster, one of the open reading frames (ORF10) has a 63.6% G+C content. Compared to those filamentous fungal genomes characterized so far, including N. crassa, A. nidulans, U. maydis (all have G+C content 51-54%, see Karlin and Mrázek, PNAS, 94, 10227, 1997), the genomic region around CPS1 is unusual. This might suggest that the gene cluster harboring CPS1 came from a bacterial source (since most bacterial genes are known to have a high G+C content), but has evolved into a fungal version.

Based on these data, CPS1 homologs may have a common ancestral gene which was acquired from a bacterial species via horizontal transfer and then maintained by the fungal genome via vertical transmission in closely related lineages.

In the evolution process, the genus Cochliobolus could also have inherited a second gene (A) controlling the ability to take up foreign DNA, by which its ancestor took the “alien” CPS1. As a result, this group of fungi is able to keep trapping genes from other organisms by additional “horizontal transfers” and giving rise to new races or even new species characterized by the ability to produce unique pathogenesis factors. The direct support for this hypothesis is that both the Tox2 locus of C. carbonum and the Tox1 locus of C. heterostrophus are associated with large fragments of “alien” DNA (A+T-rich and highly repeated) and the same could also be true for Tox3 controlling victorin production by C. victoriae, although there is yet no direct experimental evidence (Ahn and Walton, 1996, supra; Yang et al., 1996, supra; Yoder et al., 1997, supra). In contrast to CPS1, these gene transfers must have occurred in the recent evolutionary past because both Tox1 and Tox2 loci are found only in specific isolates in the species, e.g., the acquisition of Tox1 genes probably occurred as recently as the 1960s when race T was first identified in the field (Yoder et al., 1997, supra).

There are other possibilities for the evolution of CPS1. First, each genus mentioned above could have acquired CPS1 independently after divergence of the lineage. But this seems less likely because this would need to happen at the same time and involve the same donor organism if the fact that the homologs detected in Cochliobolus and Setosphaeria gave similar hybridization signal intensity is considered. Second, the horizontal transfer of CPS1 could have occurred at earlier time periods such as before the divergence of Pleosporales or even the Ascomycotina To test these hypotheses, detection of CPS1 homologs in Pyrenophora, Pleospora and other genera must be done by either genomic DNA hybridization or PCR Based on the facts discussed here, it is not unreasonable to predict that additional CPS1 homologs will be found in other fungal species. Further investigation could provide an direct entry point for understanding the evolution of fungal pathogenesis to plants.

The C. heterostrophus CPS1 gene was cloned by identification of genomic DNA fragments recovered from the tagged site in a mutant generated using REMI insertional mutagenesis. Characterization of two overlapping cosmid clones in this study has proved that no deletions or chromosome rearrangements are associated with the gene tagging event, because both cosmids carry the same fragment which span the REMI insertion site and the nucleotide sequence in this region is the same as that of recovered genomic DNA from the tagged site. This undoubtedly clarifies the identity of CPS1, which is the major biosynthetic gene. Mapping and sequencing of the two cosmids extended the sequence by 27.4 kb from the previously cloned fragment, leading to the characterization of 38.7 kb of contiguous genomic DNA, the largest genomic region analyzed so far in C. heterostrophus. In addition to CPS1 and TES1, sequence analysis of this region revealed at least 11 open reading frames; three of them, designated as DBZ1, CAT1 and DEC2, respectively, apparently encode functional proteins. The tight linkage of these genes suggests that they may be involved in the same pathway.

In filamentous fungi, in some cases, genes in pathways for biosynthesis of secondary metabolites are dispersed on different chromosomes, e.g., the cephalosporin C pathway genes in Acremonium chrysogenum (Mathison et al., Curr. Genet., 23, 33, 1993) and the melanin pathway genes in Colletotrichum lagenariun (Kubo et al., Appl. Environ. Microbiol., 62, 4340, 1996). In other cases, tightly linked genes are usually found to be functionally related to a common pathway. This clustering organization has been exemplified by the sterigmatocystin pathway genes of Aspergillus nidulans, in which 25 coordinately regulated transcripts are found in a 60 kb genomic region (Brown et al., 1996) and the trichothecene pathway genes of Fusarium sporotrichioides, in which 9 genes are clustered in a 25 kb region and 8 of them have been shown to be required for the pathway function (Hohn et al., Mol. Gen. Genet., 248, 95, 1995). The genes involved in biosynthesis of certain fungal peptides are also found as clusters. The tight linkage between CPS1 and these additional genes might reveal the presence of a novel secondary metabolite pathway in C heterostrophus. In this pathway, CPS1 is the major structural gene since it encodes a large multifunctional enzyme with all catalytic activities required for synthesis of a secondary metabolite, presumably a peptide phytotoxin; other genes may carry out different functions required for coordinate operation of the pathway, such as regulation, posttranslational modification or substrate processing as discussed below.

Both functional and structural analyses strongly support the hypothesis that the CPS1 gene cluster controls a novel biosynthetic pathway. Pathway genes have been studied only in a few filamentous fungi mainly for industrial purposes (Keller et al., J. Ind. Microbiol. Biotechnol., 19, 305, 1997). For plant pathogenic fungi, little is known about pathway genes for fungal pathogenesis. In C. heterostrophus, recent cloning of two Tox1 genes PKS1 (Yang et al., 1996, supra) and DEC1 (Rose et al., 1996, supra) have contributed to a breakthrough in understanding the molecular mechanism for biosynthesis of T-toxin, a virulence determinant in the fungus/corn interaction. But further identification of related pathway genes has been unsuccessful because the two genes are located on different chromosomes and each is embedded in A+T-rich DNA (Yoder et al., 1997, supra). In contrast, the CPS1 cluster provides a good opportunity to explore a pathogenesis pathway.

First, it resides in a “normal” sequence region. G+C content of a 50-55% is found in most of the cloned sequences and no A+T-rich DNA is associated with either end of the cloned region. This would facilitate cloning of additional pathway genes by further chromosome walking, by screening of cosmid libraries or the targeted integration and plasmid rescue. Second, it contains a regulatory gene (DBZ1) which is presumably linked to a signal transduction pathway. Isolation of genes that interact with DBZ1 could reveal novel factors mediating the molecular communication between fungal pathogen and the host plant. Further characterization of DBZ1 (along with position-specific disruption or deletion) would be also helpful in determining the limit of the gene cluster, because tightly linked genes involved in a common pathway are often coordinately regulated by the same regulatory factor (Keller et al., 1997, supra). Finally, CPS1 genes are found in both race T and race O, and its homologs are also found in other Cochliobolus species. Presence of high G+C content may imply that these genes evolved from a bacterial ancestor and the conservation in these fungi may correlate with the phytopathogenic function of the gene products encoded by the CPS1 cluster. Further investigation of this cluster should provide insights into the evolution of general pathogenicity factors among this group of fungi.

Ferric reductases are a group of enzymes found in bacteria, fungi, plants and animals that are responsible for reduction of ferric iron to ferrous iron, an absorptive form used by the organism. They have been well studied in S. cervisiae, C. albicans and H. capsulatum and the like. The yeast FER1 has been expressed in tobacco (Oki et al., 1999).

Previous studies have shown that FER genes could be important pathogenic determinants. Timmerman and Woods have proposed that in H. capsulatum FER could play critical roles in the acquisition of iron in three different ways: from inorganic or organic ferric salts, from host Fe(III) binding proteins (transferrin and the like), and from siderophores produced by the fungus itself (to reduce and release the iron chelated by the siderophore molecules).

On the other hand, iron sequestration in response to microbial infection has been demonstrated to be a host defense mechanism. The infection-related iron acquisition system in the pathogen can be considered to be an important mechanism against host defense and for a successful colonization by the pathogen in the host cells. This could be a general mechanism for all pathogenic fungi.

CPS1 does encode a peptide synthetase which is responsible for biosynthesis of a novel siderophore with unusual amino acid, hydroxyl acid and architecture, which is why CPS1 does not show similarity to common NRPSs. The CPS1 siderophore can compete with the host for iron acquisition when the fungus enters its host cells where the iron is limited due to host sequestration. In particular, for root pathogens such as C. victoriae, sequestration may be stronger in the root surface. This could explain why the cps1 mutant showed drastically reduced virulence. The FER1 could be required to release iron from the CPS1 siderophore which explains its location near the CPS1 gene. Moreover, fungal strains could be cultured in iron-limiting conditions because CPS1, and likely other genes in the cluster maybe turned on only during conditions of iron depletion.

In a preferred embodiment, the polypeptides, including those having substantially similar activities to SEQ ID NO:47, SEQ ID NO:49, or SEQ ID NO:56 are encoded by nucleotide sequences derived from fungi, preferably from pathogenic fungi, desirably identical or substantially similar to the nucleotide sequences set forth in SEQ ID NO:46, SEQ ID NO:48, SEQ ID NO:55 or the complement thereof.

In another preferred embodiment, the present invention describes a method for identifying agents having the ability to inhibit or reduce the activity of any one or more of SEQ ID NO:47, SEQ ID NO:49 or SEQ ID NO:56 in fungi. Preferably, a transgenic “lockout” fungus and/or fungal cell, is obtained which preferably is stably transformed, which comprises a deletion in any of SEQ ID NO:46, SEQ ID NO:48 or SEQ ID NO:55. Thus, in one embodiment, the gene product encoded by the nucleotide sequence is not expressed, or has reduced or aberrant expression. In another embodiment, the transgenic fungus or cell comprises the corresponding non-deleted sequences linked to a promoter to yield a gene product which is overexpressed. An agent is then contacted with the transgenic fungus and/or cell, and the growth development, virulence or pathogenicity of the transgenic fungus and/or cell is determined relative to the growth, development, or pathogenicity, of the corresponding transgenic fungus and/or cell to which the agent was not applied; or to the corresponding nontransgenic fungus and/or cell.

The present invention generally relates to an isolated nucleic acid molecule from a fungal pathogen encoding a CPS1 peptide synthetase, an iron reductase or a permease/MFS trasporter. In a preferred embodiment, a DNA molecule has a nucleotide sequence which hybridizes to a DNA molecule having a sequence corresponding to SEQ ID NO:46, SEQ ID NO:48 or SEQ ID NO:55. Other DNA molecules of the present invention include DNA molecules that have a sequence which is greater than 65% identical to the nucleotide sequence of SEQ ID NO:46, SEQ ID NO: 48 or SEQ ID NO:55. Nucleotide sequence similarity is determined by the BLAST program with the default parameters (Altschul et al., “Basic Local Alignment Search Tool,” J. Mol. Biol., 215:403 (1990). Preferred sequences include those DNA molecules which will hybridize to a nucleic acid molecule having the sequence of SEQ ID NO:46, SEQ ID NO:48, SEQ ID NO:55 or the complement thereof. Preferably, the DNA molecules hybridize to SEQ ID NO:46, SEQ ID NO:48, SEQ ID NO:55, or its complement under low or moderate, or stringent conditions.

Other proteins or polypeptides of the present invention include polypeptides having an amino acid sequence which has at least 75% similarity to the amino acid sequence of SEQ ID NO:47, SEQ ID NO:49 or SEQ ID NO:56. In a preferred embodiment of the invention, the protein or polypeptide will have at least 90% similarity with SEQ ID NO:47, SEQ ID NO:49 or SEQ ID NO:56.

In addition, the nucleic acid molecules of the invention may be modified, adapted, and optimized in such a manner that, when transferred into an appropriate host cell, the modified polynucleotide confers an altered phenotype brought about by the polypeptide encoded by the modified sequence. One advantage of this method is that it can be used to rapidly evolve any protein without knowledge of its structure. Peptide synthetase, iron reductase and/or permease/MFS transporter polynucleotides can be altered using sequence-shuffling methods as described by WO 00/28008 and references therein. Peptide synthetases of the invention can be recombined with other peptide synthetases, iron reductases and/or permeases/MFS transporters to generate peptide synthetases, iron reductases and/or permeases/MFS transporters of desired and/or novel specificity and/or activity, and thus generate desired and/or novel non-encoded peptide products. Such novel peptide synthetases, iron reductases and/or permeases/MFS transporters would have at least one active domain or other desired property-imparting domain (e.g., binding, enzymatic activity, specificity determining).

Briefly, sequences or fragments of sequences are shuffled by various recombinatorial methods, the shuffled polynucleotide is introduced into a suitable host for expression, the resulting phenotype is measured and the modified phenotype is compared with the phenotype produced by unmodified sequence. Here, “phenotype” refers to the trait of interest and may include measuring the amount, conformation, composition, or enzymatic activity of the polypeptide encoded, if the sequence shuffling is being performed, to modify a single protein. Phenotype may also be assessed by measuring the effect of expression of the modified peptide synthetase, iron reductase and/or permease/MFS transporter polynucleotide on expression of other genes, on cellular processes such as respiration or glycolysis, on tissue-level processes such as cell shape and size, and on organismal traits such as pathogenicity and/or virulence. Sequence-shuffled peptide synthetase polynucleotides producing a desirable phenotype are then selected, further modified, and the resulting phenotype is measured. The shuffling and selection process is performed iteratively until sequence shuffled polynucleotides encoding at least one polypeptide producing the desired phenotype is obtained, or until optimization of the trait of interest has plateaued and no further improvement is seen in subsequence rounds of shuffling and selection. Alternately, multiple rounds of recombination of peptide synthetase sequences maybe performed prior to any selection step, with the aim of increasing the diversity of resulting populations nucleic acids prior to selection.

At least five general classes of recombination methods may be applied to peptide synthetase, iron reductase and/or permease/MFS transporter polynucleotides. First, the nucleic acids of peptide synthetase, iron reductase and/or permease/MFS transporter polynucleotides can be recombined in vitro by any of a variety of techniques including DNAse digestion of polynucleotides followed by ligation and/or PCR reassembly of the polynucleotides. Second, polynucleotides can be recursively recombined in vivo, for example by allowing recombination to occur between an introduced peptide synthetase, iron reductase and/or permease/MFS transporter polynucleotide and homologous sequences in a cell. Third, whole cell genome recombination methods can be used in which whole genomes of cells are recombined, optionally including spiking the genomic (nuclear and/or plastid) recombination mixtures with the peptide synthetase, iron reductase and/or permease/MFS transporter sequences of interest. Fourth, synthetic recombination methods can be used, in which oligonucleotides corresponding to different homologs of the peptide synthetase, iron reductase and/or permease/MFS transporter sequence are synthesized and reassembled in PCR or ligation reactions which also include oligonucleotides which correspond to more than one allelic variant, thereby generating new recombined polynucleotides. Fifth, in silico methods of recombination can be carried out in which genetic algorithms are used in a computer to recombine sequence strings which correspond to homologs of the peptide synthetase sequences of interest. The resulting recombined sequence strings are optionally converted into nucleic acids by synthesis of nucleic acids which correspond to the recombined sequences. Such synthesis could proceed by oligonucleotide synthesis and gene reassembly techniques. Any of the preceding general recombination formats can be practiced reiteratively to generate a more diverse set of recombinant nucleic acids.

The ever-increasing quantity and quality of data being accumulated not only about gene sequence, structure and function, but also about gene expression patterns and proteins interactions on genomic scales, makes it no longer feasible to deal with genetic data on an item-by-item basis but instead, necessary to create new ways of discovering biological information by in silico data mining. “Data mining” as used herein, refers to exploration and analysis of large quantities of data, by automatic and semi-automatic means, in order to discover meaningful patterns and rules. Data mining is applied to molecular sequence and structure data, gene expression and other high-throughput data, and to existing knowledge in the scientific literature, including making meaningful connections between different forms of knowledge and data.

A variety of data mining tools can be applied using the peptide synthetase, iron reductase and/or permease/MFS transporter sequences of the present invention. A method appropriate for use in sequence databases which contain long stretches of data known as long-pattern data sets, is that disclosed in U.S. Pat. No. 6,138,117, which uses a look-ahead scheme for quickly identifying long patterns that is not limited to the initialization phase, an heuristic item-ordering policy for tightly focusing the search, and a support-lower-bounding scheme that is also applicable to other algorithms. Recursive partitioning is useful to elucidate structure-activity relations and to guide decision-making for high-throughput screening of compounds for their effects on peptide synthetase polypeptides, for example as described by Hertzog et al. (J. Pharmacol Toxicol Methods 42:207 (1999)) for sequential screening of G-protein-coupled receptors. The peptide synthetase, iron reductase and/or permease/MFS transporter sequences of the present invention may be applied to digital differential display (DDD) to analyze differential expression and create an electronic expression profile for a variety of physiological conditions. Peptide synthetase, iron reductase and/or permease/MFS transporter sequence data can be analyzed to predict protein domains using the BLAST algorithm. Higher-order correlations among peptide synthetase, iron reductase and/or permease/MFS transporter proteins may be predicted by using peptide synthetase protein sequence data to compare sets of sequence-distant sites displaying high mutual information which may bespeak important structural or functional features, a methodology that overcomes the limitations of previous methods which examined only single-residue features or pairwise interactions. (Steeg et al., Pac Symp Biocomput 1998:573 (1998)).

Peptide synthetase, iron reductase and/or permease/MFS transporter polypeptide sequences having structures expressed in a computer-readable form can be evaluated for function using functional site descriptors (FSDs) for a biomolecule functional site having a specific biological function, as described in the publication WO 00/11206. FSDs can be used to identify or screen for a novel function in one or more peptide synthetase, iron reductase and/or permease/MFS transporter polypeptides, to confirm a previously identified or suspected function of a protein, to evaluation the effects of sequence shuffling on protein function, or to provide further information about a specific functional site in a peptide synthetase, iron reductase and/or permease/MFS transporter polypeptide.

FSDs are geometric representations of protein functional sites, typically defining spatial configurations of functional sites by providing a three-dimensional (3D) representation of a protein functional site. Preferred functional sites represented by FSDs include a ligand binding domain, an ion or cofactor binding site, a site or domain for protein-protein interaction, or an enzymatic active site. An FSD typically comprises a set of geometric constraints for one or more atoms in each of two or more amino acid residues comprising a function site of a protein. Geometric constraints of an FSD may comprise an atomic position specified by a set of 3D coordinates, an interatomic distance, an interatomic bond angle, or conformational constraints imposed by residues at a site or by secondary structure such as a zinc finger, leucine zipper, helix, or a strand, where these constraints may be expressed either as fixed coordinates or ranges. Libraries of FSDs can comprise at least two FSDs for at least one of the biological functions represented by the library.

FSDs are used to probe protein structures to determine if such structures contain the functional sites described by the corresponding FSDs. Peptide synthetase, iron reductase and/or permease/MFS transporter polypeptides to be screened can comprise an unmodified sequence selected from SEQ ID NO:47, SEQ ID NO:49 or SEQ ID NO:56, or a modified form derived from random or directed sequence shuffling as previously described. Typically, functional screening methods comprise applying a FSD to a structure of a peptide synthetase, iron reductase and/or permease/MFS transporter polypeptide, where the structure may be determined by x-ray crystallography, nuclear magnetic resonance, by a computer “ab initio” folding program a homology program, or a “threading” program, and expressed in a computer-readable form.

The function of a peptide synthetase, iron reductase and/or permease/MFS transporter polypeptide whose structure is expressed in computer-readable form can be screened by applying an FSD to the structure of a peptide synthetase, iron reductase and/or permease/MFS transporter polypeptide and determining whether the peptide synthetase, iron reductase and/or permease/MFS transporter polypeptide structure matches, or satisfies, the constraints of the FSD. Libraries of FSDs can be used to probe for or evaluate the activity or function associated with the FSD in one or more protein structures.

The DNA molecule encoding the CPS1, iron reductase polypeptide and/or permease/MFS transporter of the present invention can be incorporated in cells using conventional recombinant DNA technology. Generally, this involves inserting the DNA molecule into an expression system to which the DNA molecule is heterologous (i.e., not normally present). The heterologous DNA molecule is inserted into the expression system or vector in proper sense orientation and correct reading frame. The vector contains the necessary elements for the transcription and translation of the inserted protein-coding sequences. U.S. Pat. No. 4,237,224, describes the production of expression systems in the form of recombinant plasmids using restriction enzyme cleavage and ligation with DNA ligase. These recombinant plasmids are then introduced by means of transformation arid replicated in unicellular cultures including prokaryotic organisms and eukaryotic cells grown in culture. Recombinant genes may also be introduced into viruses, such as vaccinia virus. Recombinant viruses can be generated by transfection of plasmids into cells infected with virus.

Suitable vectors include, but are not limited to, the following viral vectors such as lambda vector system gt11, gtWEST.B, Charon 4, and plasmid vectors such as pBR22, pBR325, pACYC177, pACYC184, pUC8, pUC9, pUC18, pUC19, pLG339, pR290, pKC37, pKC1O1, SV40, pBluescript I SK+/−or KS +/−(see “Stratagene Cloning Systems” Catalog (1993) from Stratagene, La Jolla, Calif.), pQE, pIH821, pGEX, pET series (see Studier et. al., “Use of T7 RNA Polymerase to Direct Expression of Cloned Genes,” Gene Expression Technology, vol.185 (1990)), and any derivatives thereof. Suitable vectors are continually being developed and identified. Recombinant molecules can be introduced into cells via transformation, transduction, conjugation, mobilization, or electroporation. The DNA sequences are cloned into the vector using standard cloning procedures in the art, as described by Maniatis et al. or Sambrook et al., Molecular Cloning: A Laboratory Manual, Cold Springs Laboratory, Cold Springs Harbor, N.Y. (1982 or 1989, respectively).

A variety of host-vector systems may be utilized to express the protein-encoding sequence(s). Primarily, the vector system must be compatible with the host cell used. Host-vector systems include but are not limited to the following: bacteria transformed with bacteriophage DNA, plasmid DNA) or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria or transformed via particle bombardment (i.e., biolistics). The expression elements of these vectors vary in their strength and specificities. Depending upon the host-vector system utilized, any one of a number of suitable transcription and translation elements can be used. Different genetic signals and processing events control many levels of gene expression (e.g., DNA transcription and messenger RNA, “mRNA” translation). Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a procaryotic system, and, further, prokaryotic promoters are not recognized and do not function in eukaryotic cells. Similarly, translation of DNA in procaryotes depends upon the presence of the proper prokaryotic signals which differ from those of eukaryotes. Efficient translation of DNA in procaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 165, rRNA (ribosomal RNA) and probably promote binding of mRNA to ribosomes by duplexing with the rRNA to allow correct positioning of the ribosome. For a review on maximizing gene expression, see Koberts and Lauer, Methods in Enzymology 68:473 (1979).

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli; its bacteriophages, or plasmids, promoters such as the phage promoter, lac promoter, trp promoter, recA promoter, ribosomal RNA promoter, the PR and PL promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the insert gene. Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthiobeta-D-galactoside). A variety of other operons, such as tip, pro, etc., are under different controls. Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno (“SD” sequence about 7-9 bases 5′ to the initiation codon (“ATG”) to provide a ribosome binding site. Thus, any SD-ATG combination that can be utilized by host cell ribosomes maybe employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used. The present invention also relates to anti-sense nucleic acid for essential cell proteins, such as replication proteins which serve to tender host cells incapable of further cell growth and division. Anti-sense regulation has been described by Rosenberg et al., Nature, 313:703 (1985); Preiss et al., Nature, 313:27 (1985); Melton, Proc. Natl. Acad. Sci. USA, 82:144 (1985); Izaut et al., Science, 229:342 (1985); Kim et al., Cell, 42:129 (1985); Bestka et al., Proc Natl. Acad. Sci. USA, 81:7525 (1984); Coleman et al., Cell, 37:429 (1984); and McQany et al., Proc. Natl. Acad. Sci. USA, 83:399 (1986), which are hereby incorporated by reference.

Once the isolated DNA molecules encoding the CPS1 polypeptide or iron reductase have been cloned into an expression system, they are ready to be incorporated into a host cell. Such incorporation can be carried out by the various forms of transformation noted above, depending upon the vector host cell system. Suitable host cells include, but are not limited to, bacteria, virus, yeast, mammalian cells, insect, plant, and the like. In the present invention, the host cells are from plants such as corn, oat, grass, weeds, bamboo, and sugarcane. In this aspect of the present invention, large numbers of compounds can be screened for their activity as inhibitors of CPS1 protein, iron reductase or permease/MFS transporter by a high throughput screening assay as described in U.S. Pat. No. 5,767,946. Generally, a library of compounds is assayed for inhibition of an enzyme catalyzed reaction and the amounts of fluorescence bound to individual suspendable solid supports measured to determine the degree of inhibition. For example, the amount of fluorescence bound to a microbead in the presence of inhibitory compounds is greater than for non-inhibitory compounds. The amounts of fluorescence bound to individual beads are determined by confocal microscopy. Using this type of assay, inhibition can be determined, e.g., of a peptide synthetase such as CPS1. For CPS1 the substrate can be amino acids (or hydroxy acids), linked at one end to the microbead and at the other end to a fluorescent label. The enzyme inhibitors can be utilized to impart fungal resistance to a variety of vertebrate organisms.

Another aspect of the present invention involves using one or more of the above DNA molecules encoding the CPS1 polypeptide or a gene encoding an enzyme that degrades the CPS1 product to transform organisms to impart fungal resistance to the organism. This concept of pathogen-derived resistance, according to U.S. Pat. No. 5,840,481 is that host resistance to a particular parasite can effectively be engineered by introducing a gene, gene fragment, or modified gene or gene fragment of the pathogen into the host. This approach is based on the fact that in any parasite-host interaction, there are certain parasite-encoded cellular functions (activities) that are essential to the parasite but not to the host and that when one of the essential functions of the parasite such as survival or reproduction is disrupted, the parasitic process will be stopped. “Disruption” refers to any change that diminishes the survival, reproduction, or ineffectivity of the parasite. Such essential functions, which are under the control of the parasite's genes, can be disrupted by the presence of a corresponding gene product in the host which is (1) dysfunctional, (2) in excess, or (3) appears in the wrong context or at the wrong developmental stage in the parasite's life cycle. If such faulty signals are designed specifically for parasitic cell functions, they will have little effect on the host. Therefore, the procedure for making organisms, for example, resistant to infection by one or more fungus involve isolating DNA coding for a gene such as CPS1 of a fungus, operably linking the DNA within an expression vector; and transforming a cell or tissue with the expression vector. The transformed cells or tissue in the presence of the fungus such as Cochliobolus heterostrophus where the CPS1 DNA is expressed as a gene product and the CPS protein disrupts the essential activity of the fungi.

Dosages, Formulations and Routes of Administration of the Agents of the Invention

The therapeutic agents identified by the methods of the invention may be administered at dosages of at least about 0.01 to about 100 mg/kg, more preferably about 0.1 to about 50 mg/kg, and even more preferably about 0.1 to about 30 mg/kg, of body weight, although other dosages may provide beneficial results. The amount administered will vary depending on various factors including, but not limited to, the agent chosen, the disease, whether prevention or treatment is to be achieved, and if the agent is modified for bioavailability and in vivo stability.

Administration of a sense or antisense nucleic acid molecule encoding a therapeutic agent may be accomplished through the introduction of cells transformed with an expression cassette comprising the nucleic acid molecule (see, for example, WO 93/02556) or the administration of the nucleic acid molecule (see, for example, Felgner et al., U.S. Pat. No. 5,580,859, Pardoll et al., Immunity, 3:165 (1995); Stevenson et al., Immunol. Rev., 145:211 (1995); Molling, J. Mol. Med., 75:242 (1997); Donnelly et al., Ann. N.Y. Acad. Sci., 772:40 (1995); Yang et al., Mol. Med. Today, 2:476 (1996); Abdallah et al., Biol. Cell, 85:1 (1995)). Pharmaceutical formulations, dosages and routes of administration for nucleic acids are generally disclosed, for example, in Felgner et al., supra.

The therapeutic agents of the invention are amenable to chronic use for prophylactic purposes, preferably by systemic administration.

Administration of the therapeutic agents in accordance with the present invention may be continuous or intermittent, depending, for example, upon the recipients physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated.

One or more suitable unit dosage forms comprising the therapeutic agents of the invention, which, as discussed below, may optionally be formulated for sustained release, can be administered by a variety of routes including oral, or parenteral, including by rectal, buccal, vaginal and sublingual, transdermal, subcutaneous, intravenous, intramuscular, intraperitoneal, intrathoracic, intrapulmonary and intranasal routes. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for oral administration, they are preferably combined with a pharmaceutically acceptable carrier, diluent or excipient to form a pharmaceutical formulation, or unit dosage form. The total active ingredients in such formulations comprise from 0.1 to 99.9% by weight of the formulation. By “pharmaceutically acceptable” it is meant the carrier, diluent, excipient, and/or salt must be compatible with the other ingredients of the formulation, and not deleterious to the recipient thereof. The active ingredient for oral administration may be present as a powder or as granules; as a solution, a suspension or an emulsion; or in achievable base such as a synthetic resin for ingestion of the active ingredients from a chewing gum. The active ingredient may also be presented as a bolus, electuary or paste.

Formulations suitable for vaginal administration may be presented as pessaries, tampons, creams, gels, pastes, douches, lubricants, foams or sprays containing, in addition to the active ingredient, such carriers as are known in the art to be appropriate. Formulations suitable for rectal administration may be presented as suppositories.

Pharmaceutical formulations containing the therapeutic agents of the invention can be prepared by procedures known in the art using well-known and readily available ingredients. For example, the agent can be formulated with common excipients, diluents, or carriers, and formed into tablets, capsules, suspensions, powders, and the like. Examples of excipients, diluents, and carriers that are suitable for such formulations include the following fillers and extenders such as starch, sugars, mannitol, and silicic derivatives; binding agents such as carboxymethyl cellulose, HPMC and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

For example, tablets or caplets containing the agents of the invention can include buffering agents such as calcium carbonate, magnesium oxide and magnesium carbonate. Caplets and tablets can also include inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, and zinc stearate, and the like. Hard or soft gelatin capsules containing an agent of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric coated caplets or tablets of an agent of the invention are designed to resist disintegration in the stomach and dissolve in the more neutral to alkaline environment of the duodenum.

The therapeutic agents of the invention can also be formulated as elixirs or solutions for convenient oral administration or as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol”, polyglycols and polyethylene glycols, C₁-C₄ alkyl esters of short-chain acids, preferably ethyl or isopropyl lactate, fatty acid triglycerides such as the products marketed under the name “Miglyol”, isopropyl myristate, animal, mineral and vegetable oils and polysiloxanes.

The compositions according to the invention can also contain thickening agents such as cellulose and/or cellulose derivatives. They can also contain gums such as xanthan, guar or carbo gum or gum arabic, or alternatively polyethylene glycols, bentones and montmorillonites, and the like.

It is possible to add, if necessary, an adjuvant chosen from antioxidants, surfactants, other preservatives, film-forming, keratolytic or comedolytic agents, perfumes and colorings. Also, other active ingredients may be added, whether for the conditions described or some other condition.

For example, among antioxidants, t-butylhydroquinone, butylated hydroxyanisole, butylated hydroxytoluene and á-tocopherol and its derivatives may be mentioned. The galenical forms chiefly conditioned for topical application take the form of creams, milks, gels, dispersion or microemulsions, lotions thickened to a greater or lesser extent, impregnated pads, ointments or sticks, or alternatively the form of aerosol formulations in spray or foam form or alternatively in the form of a cake of soap.

Additionally, the agents are well suited to formulation as sustained release dosage forms and the like. The formulations can be so constituted that they release the active ingredient only or preferably in a particular part of the intestinal or respiratory tract, possibly over a period of time. The coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, and the like.

The therapeutic agents of the invention can be delivered via patches for transdermal administration. See U.S. Pat. No. 5,560,922 for examples of patches suitable for transdermal delivery of a therapeutic agent. Patches for transdermal delivery can comprise a backing layer and a polymer matrix which has dispersed or dissolved therein a therapeutic agent, along with one or more skin permeation enhancers. The backing layer can be made of any suitable material which is impermeable to the therapeutic agent. The backing layer serves as a protective cover for the matrix layer and provides also a support function. The backing can be formed so that it is essentially the same size layer as the polymer matrix or it can be of larger dimension so that it can extend beyond the side of the polymer matrix or overlay the side or sides of the polymer matrix and then can extend outwardly in a manner that the surface of the extension of the backing layer can be the base for an adhesive means. Alternatively, the polymer matrix can contain, or be formulated of, an adhesive polymer, such as polyacrylate or acrylate/vinyl acetate copolymer. For long-term applications it might be desirable to use microporous and/or breathable backing laminates, so hydration or maceration of the skin can be minimized.

Examples of materials suitable for making the backing layer are films of high and low density polyethylene, polypropylene, polyurethane, polyvinylchloride, polyesters such as poly(ethylene phthalate), metal foils, metal foil laminates of such suitable polymer films, and the like. Preferably, the materials used for the backing layer are laminates of such polymer films with a metal foil such as aluminum foil. In such laminates, a polymer film of the laminate will usually be in contact with the adhesive polymer matrix.

The backing layer can be any appropriate thickness which will provide the desired protective and support functions. A suitable thickness will be from about 10 to about 200 microns.

Generally, those polymers used to form the biologically acceptable adhesive polymer layer are those capable of forming shaped bodies, thin walls or coatings through which therapeutic agents can pass at a controlled rate. Suitable polymers are biologically and pharmaceutically compatible, nonallergenic and insoluble in and compatible with body fluids or tissues with which the device is contacted. The use of soluble polymers is to be avoided since dissolution or erosion of the matrix by skin moisture would affect the release rate of the therapeutic agents as well as the capability of the dosage unit to remain in place for convenience of removal.

Exemplary materials for fabricating the adhesive polymer layer include polyethylene, polypropylene, polyurethane, ethylene/propylene copolymers, ethylene/ethylacrylate copolymers, ethylene/vinyl acetate copolymers, silicone elastomers, especially the medical-grade polydimethylsiloxanes, neoprene rubber, polyisobutylene, polyacrylates, chlorinated polyethylene, polyvinyl chloride, vinyl chloride-vinyl acetate copolymer, crosslinked polymethacrylate polymers (hydrogel), polyvinylidene chloride, poly(ethylene terephthalate), butyl rubber, epichlorohydrin rubbers, ethylenvinyl alcohol copolymers, ethylene-vinyloxyethanol copolymers; silicone copolymers, for example, polysiloxane-polycarbonate copolymers, polysiloxane-polyethylene oxide copolymers, polysiloxane-polymethacrylate copolymers, polysiloxane-alkylene copolymers (e.g., polysiloxane-ethylene copolymers), polysiloxane-alkylenesilane copolymers (e.g., polysiloxane-ethylenesilane copolymers), and the like; cellulose polymers, for example methyl or ethyl cellulose, hydroxy propyl methyl cellulose, and cellulose esters; polycarbonates; polytetrafluoroethylene; and the like.

Preferably, a biologically acceptable adhesive polymer matrix should be selected from polymers with glass transition temperatures below room temperature. The polymer may, but need not necessarily, have a degree of crystallinity at room temperature. Cross-linking monomeric units or sites can be incorporated into such polymers. For example, cross-linking monomers can be incorporated into polyacrylate polymers, which provide sites for cross-linking the matrix after dispersing the therapeutic agent into the polymer. Known crosslinking monomers for polyacrylate polymers include polymethacrylic esters of polyols such as butylene diacrylate and dimethacrylate, trimethylol propane trimethacrylate and the like. Other monomers which provide such sites include allyl acrylate, allyl methacrylate, diallyl maleate and the like.

Preferably, a plasticizer and/or humectant is dispersed within the adhesive polymer matrix. Water-soluble polyols are generally suitable for this purpose. Incorporation of a humectant in the formulation allows the dosage unit to absorb moisture on the surface of skin which in turn helps to reduce skin irritation and to prevent the adhesive polymer layer of the delivery system from failing.

Therapeutic agents released from a transdermal delivery system must be capable of penetrating each layer of skin. In order to increase the rate of permeation of a therapeutic agent, a transdermal drug delivery system must be able in particular to increase the permeability of the outermost layer of skin, the stratum corneum, which provides the most resistance to the penetration of molecules. The fabrication of patches for transdermal delivery of therapeutic agents is well known to the art.

For administration to the upper (nasal) or lower respiratory tract by inhalation, the therapeutic agents of the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the therapeutic agent may be administered via nose drops, a liquid spray, such as via a plastic bottle atomizer or metered-dose inhaler. Typical of atomizers are the Mistometer (Wintrop) and the Medihaler (Riker).

The local delivery of the therapeutic agents of the invention can also be by a variety of techniques which administer the agent at or near the site of disease. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

For topical administration, the therapeutic agents may be formulated as is known in the art for direct application to a target area. Conventional forms for this purpose include wound dressings, coated bandages or other polymer coverings, ointments, creams, lotions, pastes, jellies, sprays, and aerosols, as well as in toothpaste and mouthwash, or by other suitable forms, e.g., via a coated condom. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-25% by weight.

When desired, the above-described formulations can be adapted to give sustained release of the active ingredient employed, e.g., by combination with certain hydrophilic polymer matrices, e.g., comprising natural gels, synthetic polymer gels or mixtures thereof.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic agent may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; mouthwashes comprising the composition of the present invention in a suitable liquid carrier; and pastes and gels, e.g., toothpastes or gels, comprising the composition of the invention.

The formulations and compositions described herein may also contain other ingredients such as antimicrobial agents, or preservatives. Furthermore, the active ingredients may also be used in combination with other therapeutic agents, for example, oral contraceptives, bronchodilators, anti-viral agents, steroids and the like.

The invention will be further described by the following non-limiting examples.

### EXAMPLE 1

### Mutant Preparation and Characterization

Materials and Methods

Strains, Media, Crosses and Transformation. C4 (Tox1⁺; MAT-2) and C5 (Tox1⁻; MAT-1) are members of near-isogenic C. heterostrophus strains (Leach et al., 1982, supra). R.C4.2696 (Tox⁺; MAT-2; hygB^{R}) is a C4-derived mutant generated using the REMI mutagenesis procedure (Lu et al., Proc. Natl. Acad. Sci. USA 91:12649 (1994)). Strains 1301R33 (Tox⁻; MAT-2; hygB^{R}), 1301R45 (Tox⁻; MAT-1; hygB^{R}) 1301β26 (Tox⁺; MAT-2; hygB^{R}) are progeny of the cross CS X R.C4.2696. Culture media, including CM (complete medium), CMX (complete medium with xylose instead of glucose), CMNS (CM with salts omitted), and MM (minimal medium) have been described, as have mating procedures (Leach et al., 1982, supra; Turgeon et al., Mol. Gen. Genet., 201:450 (1985)). All strains were grown at 24° C. under the warm white light or black light (F40/350BL) (Sylvania Inc., Danvers, Mass.). Ascospore germination was done at 32° C. in the dark for 3 days. REMI transformants were purified by transferring the transformants from the original REMI plates to fresh CMNS medium containing hygromycin B (Calbiochem^{R}) at 80 ì g/ml. For conidiation, stable transformants were transferred to CMX containing the same drug but at a higher concentration (120 ì g/ml) to compensate for reduced drug activity due to the inhibition by the salts in the medium. Single conidia were picked up under a dissecting microscope and grown on CMNS hygromycin B plates; stable colonies were then transferred to individual CMX/hygromycin plates. All purified transformants were stored at −70° C. in CM liquid medium containing 25% of glycerol in 96-well microtiter dishes.

Bioassays. Fungal strains were grown on CMX plates (100×15 mm) for 7-10 days at 24° C. under the light for maximum conidiation. To verify normal T-toxin production by a race T isolate, 1.0 ml of T-toxin-sensitive E. coli (DHSa) cells were evenly spread on LB medium containing ampicillin (100 ì g/ml) and the plates were allowed to air dry for 30 minutes in a laminar hood. Agar plugs bearing fungal mycelia were inoculated (upside down) onto the E. coli cell lawn and the plates were incubated at 32° C. Wild type race T and race O were used as controls for each assay plate. T-toxin-producing strains of the fungus will inhibit growth of the E. coli cells and produce halos. Tox⁻ mutants can be distinguished from wild type by failure to produce a halo (tight) or by production of halos smaller (leaky) or larger than wild type (overproducing). All Tox⁻ mutants were transferred to Fries medium (Pringle et al., Phytopathology 47:369 (1957)), which optimizes toxin production, and retested.

T-cytoplasm corn plants (inbred W64A) are used to verify the Tox⁻ mutants identified from the E. coli assay using the procedure described below. Mutants defective in T-toxin production fail to produce typical race T symptoms on T-corn. Pathogenicity phenotype on N-cytoplasm corn and virulence of Tox⁺ strains to T-cytoplasm corn were determined by a plant assay where, about 3,000 transformants generated using the REMI mutagenesis procedure (Lu et al., Proc. Natl. Acad. Sci. USA, 91:12649 (1994)) were screened for mutants defective in ability to cause disease on corn plants. Two week old N-cytoplasm corn plants (inbred W64A) grown in the green house (5-6 plants in one 4″×6″ pot) were inoculated with 5 ml conidial suspensions (10⁵ conidia/ml) using a pressurized Preval Spray Gun Power Unit thin layer chromatography sprayer (Alltech Associates, Deerfield, Ill.), incubated in the mist chamber for 24 hours (23° C.) and then taken to the growth chamber (23° C., 80% humidity, 14 hours of light). The mutant phenotypes were determined by occurrence of apparent variations in disease symptom development, mainly by lesion size comparison. Mutants producing lesions smaller than wild type were retested and lengths of typical lesions from each mutant were compared with wild type 7 days after inoculation and measurements were taken for statistical evaluation.

DNA manipulations and sequencing Genomic and plasmid DNA preparation, restriction enzyme digestions, gel electrophoresis and gel blot analysis were done using standard protocols (Sambrook et al., Molecular Cloning: A Laboratory Manual, 2nd Ed, Cold Spring Harbor, N.Y.:Cold Spring Harbor Laboratory Press (1989)). DNA was sequenced at the Cornell DNA Sequencing Facility using TaqCycle automated sequencing with DyeDeoxy terminators (Applied Biosystems, Foster City, Calif.). pUCATPH was used for subcloning (Table 1). Primers used for sequencing (Table 2) were designed using Primer Select (DNASTAR Inc., LaserGene System) and synthesized by the Cornell Oligonucleotide Synthesis Facility. Sequencing of each plasmid clone was initiated with vector-specific primers or primers designed to previously determined sequences. Sequences obtained were analyzed using the same system and nucleotide or protein database searches were performed with the BLAST program (Altschul et al., J. Mol. Biol., 215:403 (1990)).

TABLE 1
Transformation vectors and clones used.

| | Length | Characteristics (See U.S. application Ser. Nos. |
|---|---|---|
| Plasmid | (kb)^{a} | 60/252,649 and 60/252,732) |
| pUCATPH | 5.1 | See FIG. 14 in U.S. application |
| | | Serial No. 60/252,649. |
| PUCATPHN | 4.6 | Cloning vector, same as pUCATPH |
| | | but lacking a 420 bp NarI fragment |
| | | containing the HindIII site |
| p214B7 | 9.2 | A clone containing pUCATPH |
| | | recovered from the tagged site in |
| | | mutant R.C4.2696 by religation of |
| | | BglII-digested genomic DNA |
| p214M1 | 6.3 | As above but with MscI-digested |
| | | genomic DNA |
| p214S1 | 9.3 | As above but with SacI-digested |
| | | genomic DNA |
| p214S1N | 3.3 | NarI fragment derived from 214S1 |
| | | containing a 0.8 kb NarI-SacI |
| | | fragment of genomic DNA ligated to |
| | | pUC18 |
| p214SNP | 8.4 | Vector for targeted integration |
| | | constructed by ligating HindIII- |
| | | digested pUCATPH into the HindIII |
| | | site of p214S1N |
| p118BSP | 7.3 | Vector for targeted integration |
| | | constructed by ligation of a 2.2 kb |
| | | SacI fragment of p118BC4 into the |
| | | SacI site of pUCATPH |
| p118BCS | 5.4 | Vector for targeted integration |
| | | constructed by ligation of a 0.8 kb |
| | | SspI fragment of p118BC4 into the |
| | | SspI site of pUCATPHN |
| p118B14 | 10.4 | A clone recovered from the p214SNP |
| | | integration site in |
| | | transformant #f118 by ligation of a |
| | | BglII-digested genomic DNA |
| | | fragment containing the entire vector |
| p118BC4 | 6.7 | A clone recovered from same site as |
| | | above but by ligation of a BclI- |
| | | digested genomic DNA fragment |
| | | containing part of vector (214SNP) |
| | | sequence |
| p9P2 | 7.3 | A clone recovered from the p118BSP |
| | | integration site in transformant #9 by |
| | | ligation of a PstI-digested genomic |
| | | DNA fragment containing pUC18 |
| p12H6 | 8.0 | A clone recovered from the |
| | | p118BCS integration site in |
| | | transformant #12 by ligation of a |
| | | HindIII-digested genomic DNA |
| | | fragment containing the entire |
| | | vector. |

^{a}An underlined kb number indicates that the plasmid carries genomic DNA sequences.

TABLE 2
Primers used for sequencing recovered genomic DNA
flanking the REMI insertion site at the R.C4
2696 mutation.

| Name^{a} | Position^{b} | Sequence^{c} | Plasmid^{d} | Origin^{e} |
|---|---|---|---|---|
| M13RMT | | SEQ ID NO: 4 | A | pUC18 |
| 1. RP1b | 775 | SEQ ID NO: 5 | A | 214B7TrpC |
| 2. RP2 | 604 | SEQ ID NO: 6 | A | 214B7RP1b |
| 3. RP3 | 119 | SEQ ID NO: 7 | A | 214B7RP2 |
| 4. RP4 | −232 | SEQ ID NO: 8 | A | 214B7RP3 |
| 5. RP5 | −812 | SEQ ID NO: 9 | A | 214B7RP4 |
| 6. RP5b | −1215 | SEQ ID NO: 10 | A | 214B7RP4 |
| 7. RP6 | −1392 | SEQ ID NO: 11 | A | 214B7RP5 |
| 8. RP7 | −1839 | SEQ ID NO: 12 | A | 214B7RP6 |
| TrpC | | SEQ ID NO: 13 | A | PUCATPH |
| 9. FP1 | 1885 | SEQ ID NO: 14 | A | 214B7TrpC |
| 10. FP1b | 1828 | SEQ ID NO: 15 | B | 214B7TrpC |
| 11. FP2 | 2028 | SEQ ID NO: 16 | B | 214M1FP1b |
| 12. FP3 | 2490 | SEQ ID NO: 17 | C | 214M1FP2 |
| 13. FP4 | 2949 | SEQ ID NO: 18 | C | 214S1FP3 |
| 14. FP4B | 2745 | SEQ ID NO: 19 | C | 214S1FP4 |
| 15. FP5 | 3421 | SEQ ID NO: 20 | C | 214S1FP4 |
| 16. FP6 | 3948 | SEQ ID NO: 21 | C | 214S1FP5 |
| 17. FP7 | 4411 | SEQ ID NO: 22 | C, D | 214S1FP6 |
| 18. FP8 | 5035 | SEQ ID NO: 23 | D | 118B14FP7 |
| 19. FP9 | 5457 | SEQ ID NO: 24 | | 118BC4FP8 |
| 20. RP48 | 2865 | SEQ ID NO: 25 | D | 214S1FP6 |
| 21. FP10 | 5790 | SEQ ID NO: 26 | F | 9P2FP9 |
| 22. FP11 | 6327 | SEQ ID NO: 27 | F | 9P2FP10 |
| 23. FP11b | 6211 | SEQ ID NO: 28 | F | 9P2FP10 |
| 24. FP12 | 6457 | SEQ ID NO: 29 | F | 9P2FP11 |
| 25. FP13 | 6854 | SEQ ID NO: 30 | F | 9P2FP12 |
| 26. FP14 | 7400 | SEQ ID NO: 31 | F | 9P2FP13 |
| 27. FP15 | 7771 | SEQ ID NO: 32 | F | 9P2FP14 |
| 28. FP16 | 8145 | SEQ ID NO: 33 | F | 9P2FP15 |
| 29. FP17 | 8492 | SEQ ID NO: 34 | F | 9P2FP16 |
| M13F40 | | SEQ ID NO: 35 | G | pUC18 |
| 30. RP1 | 8953 | SEQ ID NO: 36 | G | 9P5M13F4 |
| 31. RP2 | 8559 | SEQ ID NO: 37 | G | 9P5RP1 |
| ^{a}“RP” indicates reverse primer; “FP” indicates forward primer. Primers designed to genomic DNA sequences are numbered in order. Primers 1-17 have a leading number “214”; 18-20 with “118”; 21-29 with “9P2” and 30-31 with “9P5”. M13RMT (a M13R mutant version; there is a mutation in the polylinker of pUC18) and M13F-40 were provided by Cornell DNA Sequencing Facility. TrpC primer site is in the pUCATPH TrpC promoter | | | | |
| #region 38 bp from SaII site with sequencing direction from SaII to KpnI. | | | | |

^{b}The position of the first base of each primer corresponds to the assembled sequence (CPS1 + TES1, total 11.3 kb).
^{c}Each primer sequence is given in the 5′ to 3′ direction.
^{d}Plasmids used as templates for each sequencing reaction. A = p214B7; B = P214M1; C = p214S1; D = p118B14; E = p118BC4; F = p9P2; G = p9P5 (= 9P2)
^{e}Original sequences that were used for primer design.

Results

Recovery of tagged DNA from the REMI insertion site and targeted gene disruption. Genomic DNA of mutant R.C4.2696 was digested with BglII, MscI (no sites in pUCATPH) or SacI (which cuts the vector once) and purified by phenol extraction and ethanol precipitation, then dissolved in TE (pH 8.0). Ligation was performed in 50 μl reaction mixture, containing 1×T4 DNA ligase buffer with 10 mM ATP, 60 units T4 DNA ligase (New England Biolabs, Beverly, Mass.) and 3 μg of BglII-digested genomic DNA, at 14° C. overnight. Ten μl of ligation mixture was used to transform 200 μl of competent DH5α cells, prepared using the calcium chloride treatment (Sambrook et al., 1989, supra) to ampicillin resistance. Ampicillin resistant clones were analyzed by digestion of plasmid DNA with several diagnostic restriction enzymes and clones containing the REMI vector plus flanking genomic DNA were sequenced using the vector-specific primers (M13R or TrpC). Three plasmids, p214B7, p214MI and p214S1 were recovered and used for sequencing. p214B7 contains 4.2 kb flanking DNA (3.4 left; 0.7 right); p214M1 contains 0.1 kb left flank that overlaps with p214B7 and 1.1 kb right flank that overlaps with p214S1, which contains 3.2 kb flanking DNA on the left only.

For targeted gene disruption in wild type, p214B7 was amplified and plasmid DNA purified by equilibrium centrifugation in CsCl-ethidium bromide gradients (Sambrook et al., 1989, supra). Thirty μg of plasmid DNA (linearized with BglII for double crossover integration) were used to transform wild type and the transformants were purified by isolation of single conidia, assayed for pathogenicity and characterized by gel blot analysis.

Sequence extension by targeted integration and plasmid rescue. Two overlapping cosmid clones were isolated by probing a genomic DNA library of C4 constructed on a cosmid vector, but both extended into the left region only of p214B7. To extend to the right, a chromosome walking strategy was employed. Three targeted gene disruption experiments (each followed by plasmid rescue) were done successively. In the first experiment, a vector was constructed as follows: p214S1 was digested with NarI and religated to create p214S1N, which was then digested with HindIII and ligated into the HindIII site of pUCATPH to create p214SNP for transformation of race O (C5). One transformant (Tx118) resulting from homologous integration (confirmed by gel blot analysis) was used for plasmid rescue as described above. Two new plasmids p118B14 and p118BC4 were recovered, both of which carry sequence at the 3′ end but only 172 and 680 bp more than p214S1, respectively. To continue the walk, p118B14 was digested with SacI and ligated into the SacI site of pUCATPH to create p118BSP. This vector was linearized with BglII and transformed into wild type and one plasmid, p9P2 was recovered (from transformant Tx9), which extends 4.4 kb into the region 3′ of p118BC4 and contains the 3′ end of CPS1. The recovered plasmid p9P2 includes the entire pUC18 sequence on p118BSP and 4.6 kb of genomic DNA that contains all of ORF1 (CPS1), including the stop codon (TAG) and 3.0 kb of genomic region 3′ of the stop codon. A third experiment was done in an attempt to recover a 15 kb XhoI fragment at the 3′ end of that tagged gene. p118BCS was constructed by subcloning a 0.8 kb SspI fragment into the same site pUCATPHN. Plasmid rescue using XhoI digested-genomic DNA of a transformant (TX12) failed to recover the 15 kb XhoI fragment, but p12H6 was recovered using HindIII-digested genomic DNA of the same transformant; the genomic DNA matched that already cloned on p9P2.

Characterization of the REMI mutant. In all culture conditions used, mutant R.C4.2696 grew just like wild type with no variations in growth rate, color and morphological features. It produces normal appressorium-forming conidia that germinate and form infection structures like wild type when induced on artificial surfaces and shows normal mating ability when crossed to wild type testers. No pleiotropic phenotypes associated with the mutation have been detected so far. The mutant differs from wild type in the ability to cause disease on corn plants.

The lengths of 100 typical lesions from corn leaves inoculated with wild type race O and a mutant progeny R45 (Tox⁻, hygB^{R}) carrying the R.C4.2696 mutation were measured 7 days after inoculation and values plotted.

When tested on T-cytoplasm corn, the mutant produces race T type symptoms but the disease develops more slowly than with wild type although it produces wild type levels of T-toxin as detected in a microbial assay, suggesting that the reduced virulence is not related to a deficiency in the ability to produce T-toxin. This is clearer on N-cytoplasm corn where the mutant produces lesions significantly smaller than those produced by wild type. When the mutant was crossed to a wild type race O tester, the small lesion phenotype and ability to produce T-toxin segregated independently, indicating that mutant phenotype is not associated with the reduced fitness trait tightly linked with the Tox1 locus (Klittich et al., Phytopathology 76:1294 (1986)). The statistical evaluation of lesion size in the wild type race O genetic background indicates that the mutation causes 60% reduction in the fungal virulence to corn plants. Table 3 depicts the statistical analysis that 86% of the mutant lesions are less than 4 mm in length (average size of 3.5 mm), 60% reduced compared to that of wild type (8.5 mm).

TABLE 3

| | |
|---|---|
| Frequency | Lesion size (mm) |

| Strain | 1-4 | 5-8 | 9-12 | Mean | SD | |
|---|---|---|---|---|---|---|
| WT | 0 | 52 | 48 | 8.5 | 1.0 | A* |
| R45 | 86 | 14 | 0 | 3.5 | 0.9 | B |

*Significant difference at P < 0.01.

The mutant phenotype is caused by a tagged, single site mutation. In crosses between the mutant and wild type testers, progeny segregated 1:1 for parental types only and all hygromycin B-resistant progeny produced lesions similar to the mutant parent; all hygromycin B-sensitive progeny produced wild type lesions, indicating that a tagged mutation is responsible for the reduced pathogenicity of the mutant. Table 4 depicts the progeny segregation data

TABLE 4

| | | Parental type | | Nonparental type | |
|---|---|---|---|---|---|
| | | path | PATH | path | PATH |
| Cross | Progeny | hygB^{R} | hygB^{S} | hygB^{R} | hygB^{S} |
| R.C4.2696 x C5 | random spores | 24 | 22 | 0 | 0 |
| 1301-R33* x C5 | tetrad1 | 4 | 4 | 0 | 0 |
| | tetrad2 | 4 | 4 | 0 | 0 |
| | tetrad3 | 4 | 4 | 0 | 0 |
| | Random spores | 21 | 22 | 0 | 0 |

*13012-R33 (path, hygB^{R}, Tox⁻, MAT-2) is a progeny from the first cross, carrying the R.C4.2696 mutation.

### EXAMPLE 2

Cloning, Sequencing and Characterization of DNA Flanking the REMI Vector Insertion Site

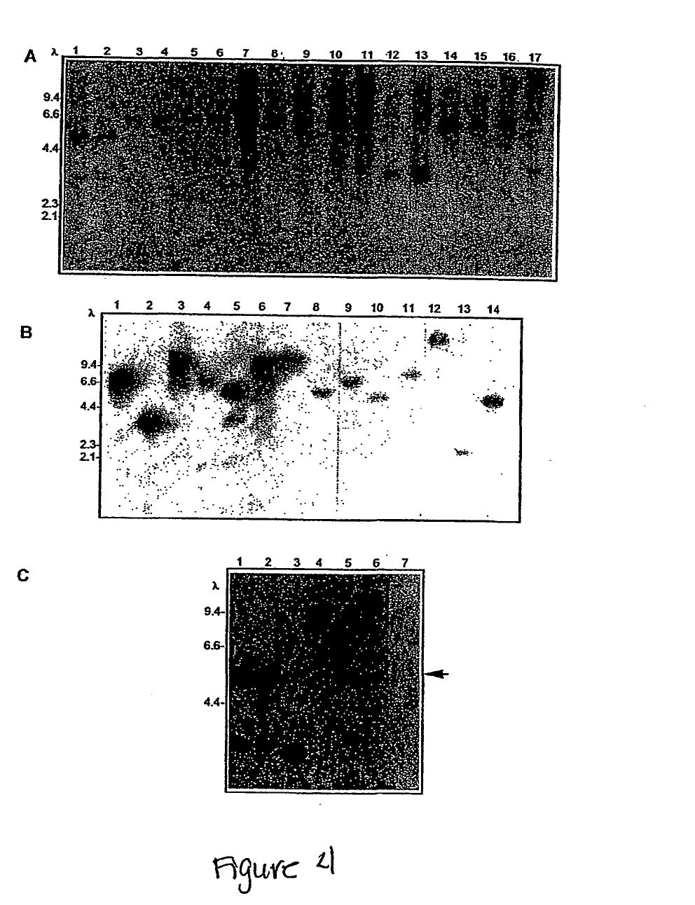

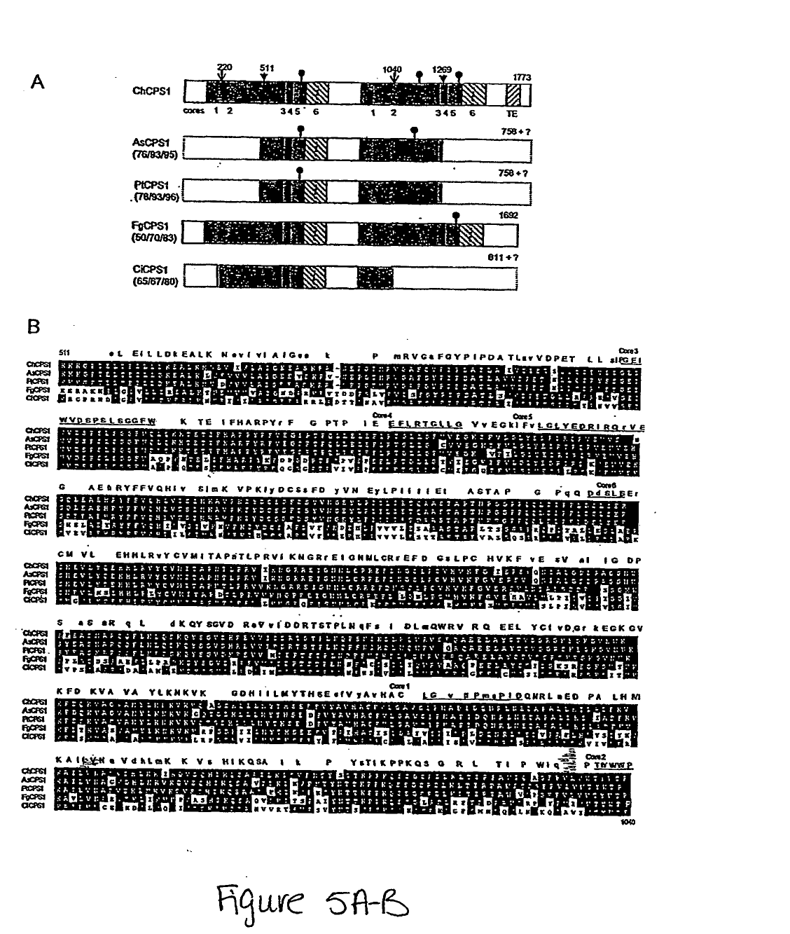

A total of 11.3 kb of genomic DNA surrounding the insertion site was cloned and completely sequenced (SEQ ID NO:59; FIG. 2). The sequence was derived from seven plasmid clones. The first three (p214B7, p214M1 and p214S1) were recovered from the tagged site in mutant R.C4.2696 and cover about 60% (6.6 kb) of the entire region. The rest (p 118B 14, p118BC4, p9P2 and p12H6) were recovered from transformants generated using the chromosome walking strategy. DNA to the left of the insertion site (3.4 kb) was cloned on p214B7; DNA on the right (7.9 kb) was cloned on different overlapping plasmids. p9P2 carries the largest amount (4.6 kb) including genomic DNA on p12H6.

Analysis of the combined sequences revealed two open reading frames (ORFs). ORF1 (5.4 kb) starts 576 bp upstream of the REMI vector insertion site and ends with an in-frame stop codon (TAG) 3029 bp from the end of the sequenced region in the right flank. No “TATA” box-like element is found in the expected position, but five putative “CAAT” boxes are located upstream of the start codon (ATG), three of them are in the range found in most filamentous fungal promoters (60-200 bp) (Gurr et al., 1987, infra). Sequence around ATG of ORF1 (CACCATGCT) (SEQ ID NO:38) is similar to the fungal consensus (CACCATGGC) (SEQ ID NO:39). Although there are several ATGs found upstream, they are less likely to be used as a start codon because the surrounding sequences lack similarity to the consensus. Three putative introns are identified by their conserved 5′ and 3′ border sequences and potential branch sites (Table 5). Splicing these introns eliminated stop codons which would otherwise interrupt the 5.4 kb open reading frame. Three introns have similar size (45-53 bp respectively) which is in the range of intron size determined from most fungal genes. A putative polyadenylation signal (ATAA) is found 223 bp downstream of the translation termination site.

The G+C content of ORF1 is 51.5%, which is similar to most Cochliobolus genes (Turgeon et al., Mol. Gen. Gene., 238:270 (1993); VanWert et al., Curr. Genet., 22:29 (1992); Yang et al., Plant Cell, 8:2139 (1996); Rose et al., 1996, supra). Interestingly, ORF1 is flanked by two regions of G+C rich DNA. The first (1.4 kb, 60.3% G+C) is found between ORF1 and ORF2; the second (1.2 kb, 60.3% G+C) is found 1.8 kb downstream of the stop codon of ORF1. Database searches using the translated protein sequence of ORF1 revealed high similarity to SafB, one of the multifunctional enzymes catalyzing the biosynthesis of the cyclic peptide antibiotic saframycin Mx1 produced by the bacterium Myxococcus xanthus (Pospiech et al., Microbiology 142:741 (1996)). The entire nucleotide sequence of ORF1 (CPS1) is designated SEQ ID NO:2 (6,550 base pairs from the 11.3 kb sequenced region, FIG. 2). The deduced amino acid sequence of CPS1 protein is designated SEQ ID NO:3. A modification of the ChCPS1 sequence, including changes in three base pairs (“ATG” added between positions 5349 and 5350 of the GenBank entry (GenBank Accession number AF332878)) and an addition of 31 amino acids (the first thirty amino acids (“MMGNYAFNPDNQQSYDGQFGSPGEASRRST”) were added at the N-terminus based on the selection of a new start codon and an additional methionine (“M” at position 1489 was missing in the Genbank entry)) is designated SEQ ID NO:50 (6553 base pairs). The deduced amino acid sequence of the modified ChCPS1 protein is designated SEQ ID NO:185 (1774 amino acids; revised version of the original CPS1 protein (GenBank Accession number AAG53991)). The open reading frame is 5,474 base pairs (736-6209), a 93 base pair increase compared to the deposited sequence that was 5,381 bp. A new start codon (position 736, the original one at position 826) was proposed based on the amino acid alignment of several CPS1 orthologs from different fingi that revealed conserved residues in this region. The stop codon (6,209) is the same as the original GenBank sequence.

TABLE 5
Characteristics of putative introns in CPS1 and
TES1

| | | Size | | | 3′ | Branch |
|---|---|---|---|---|---|---|
| Gene | Intron | (bp) | Location | 5′Border | Border | Site |
| CPS1 | I | 45 | 3060-3105 | GTAAGT | TAG | GTCTAAC |
| | II | 51 | 4532-4582 | GTAAGT | CAG | TGCTAAC |
| | III | 53 | 5187-5239 | GTACGT | CAG | TACTAAC |
| TES1 | I | 49 | 528-566 | GTAAGT | TAG | CCTTAAG |
| Cons | | | | GTA^{A}/C^{GT} | ^{T}/C^{AG} | YNCTAAC* |

ORF2 starts about 1.6 kb upstream of the start codon of CPS1 and is transcribed in the opposite direction (FIG. 2). No “TATA” box-like element and CAAT box are found; instead, an AT-rich sequence “AAAACTAT” is located 11 bp upstream of the start codon ATG and a CT motif is found in the 30 region, which is characteristic of a number of fungal genes that lack a CAAT box in their promoter region (Gurr et al., In: Gene Structure in Eukaryotic Microbes, Vol.22, published by the Society for General Microbiology, Oxford, England: IRL Press, Kinghorn, ed., pp 93-140 (1987)). The sequence around ATG matches perfectly fungal gene consensus. A putative intron (50 bp) is found in the middle of ORF2 with conserved 5′ and 3′ border sequences and a potential branch site (Table 5). A putative polyadenylation signal (AAATA) is found 189 bp downstream of the translation stop codon TGA. The G+C content of ORF2 is 55.5%, which is slightly higher than the normal range because the 5′ end of ORF2 is located in the region of G+C rich DNA upstream of ORF1. Database search revealed that ORF2 encodes a protein with high similarity to Homo sapiens thioesterase II (hTE, Liu et al., J. Biol. Chem., 272:13779 (1997)) and E. coli thioesterase II encoded by the tesB gene (Naggert et al., J. Biol. Chem., 266:11044 (1991)). The nucleotide sequence of ORF2 (TES1) is designated SEQ ID NO:57. The deduced amino acid sequence of the TES1 protein is designated SEQ ID NO:58.

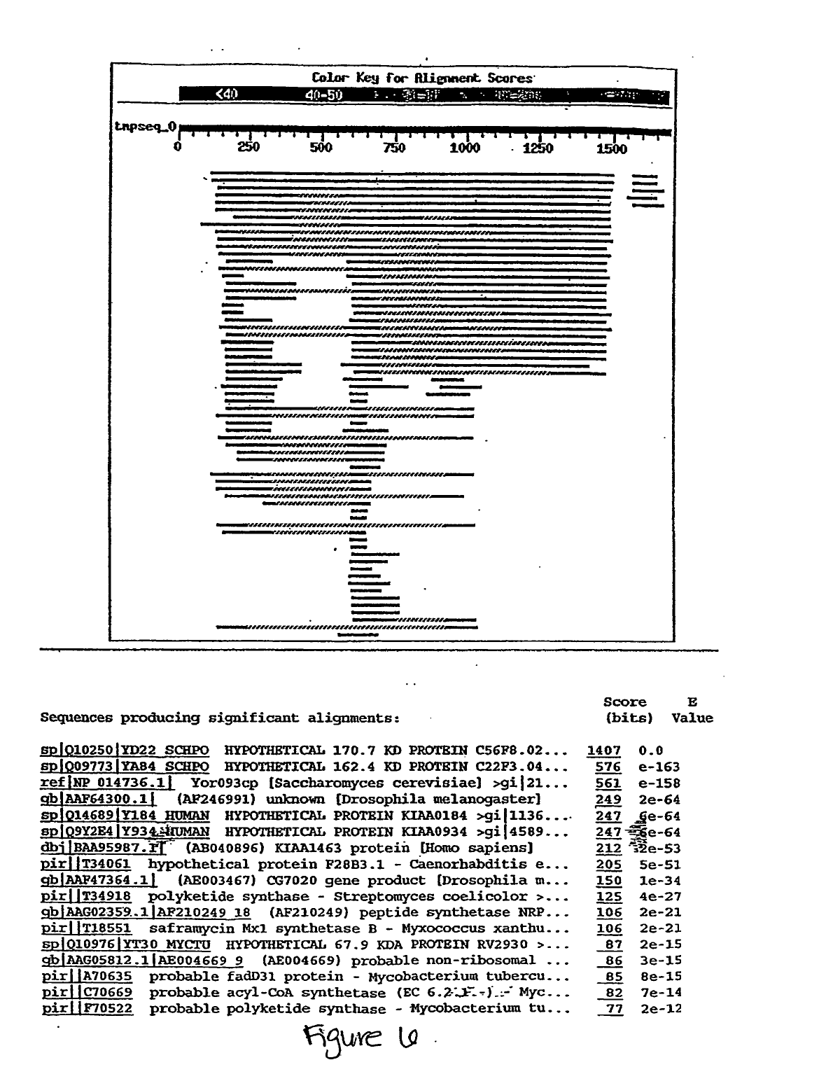

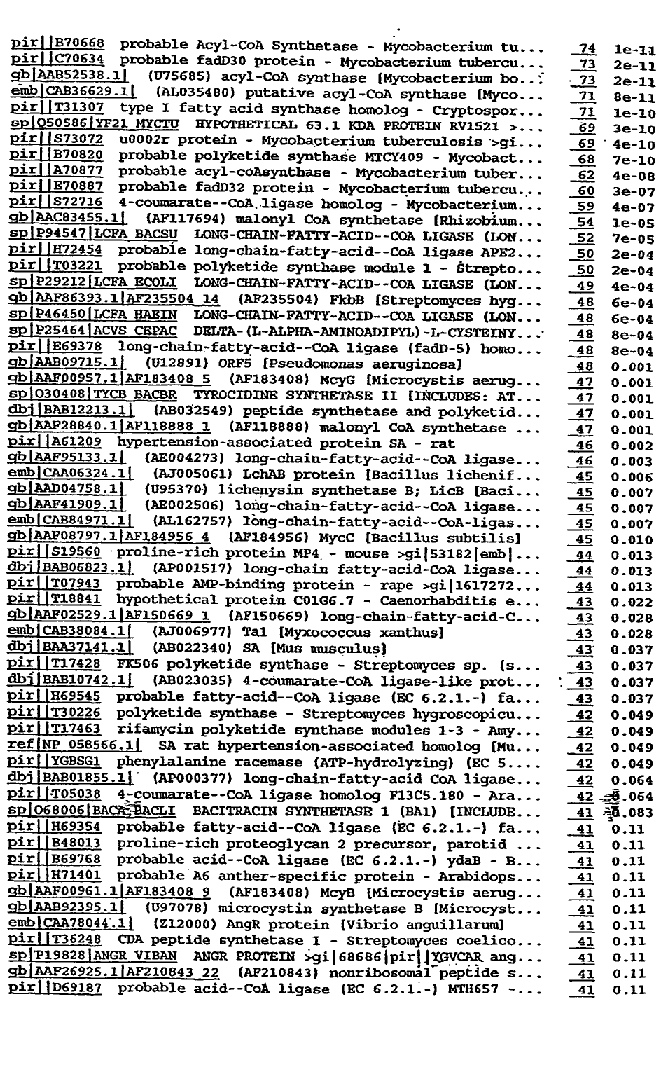

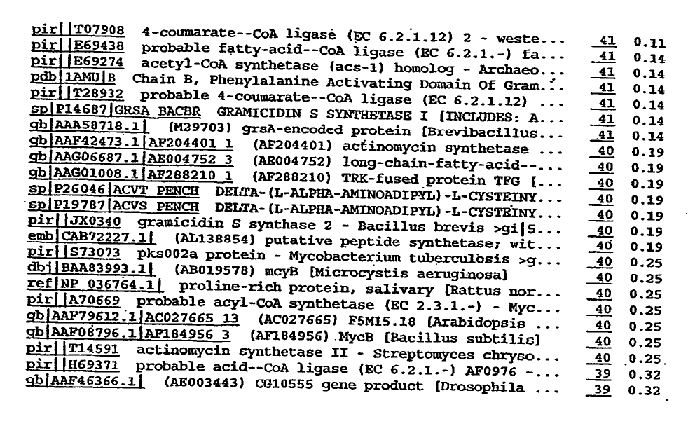

Modular structure of CPS1. Predicted CPS1 protein (1743 amino acids, Mᵣ 193235) contains two structurally similar modules, both of which are similar to SafB1, the first module of saframycin synthetase B (overall 25% identity; 50% similarity) and have apparent amino-acid-activating and thiolation domains but lack methyltransferase activity, thus appearing to be typical type I modules (FIG. 3). The number of amino acids in each module is different: the first module (CPS1A) consists of 574 amino acids (from the first residue of core 1 to the last residue of core 6), which is larger than most type I modules; the second module (CPS1B) has 530 amino acids, which is average. The distance between the two modules is 193 amino acids, much shorter than most peptide synthetases (500-600 amino acids), but this distance is not highly conserved, i.e., an opposite variation is found in HC-toxin synthetase and cyclosporine synthetase, both of which have about 1,000 amino acids between the first and second amino-acid-activating module (see Table 6F).

Tables 6A-F show a comparative alignment of core amino acid sequences in CPS1A and CPS1B with those of other peptide synthetases. In each of Tables 6A-F, the first column shows the names of peptide synthetases; the second indicates the position of the first residue aligned in the original amino acid sequence of each protein; the last column on the right indicates the number of amino acids between two cores (6A-E, in parentheses) or the distance between two adjacent amino-acid-activating modules (Table 6F, in parentheses). The extra column in 6F, shows the total number (underlined) of residues in each amino-acid-activating module in which the aligned core sequence is located. The consensus of each core sequence is on the top, which includes identical or similar residues found in all peptide synthetases or with only a few exceptions (active site also indicated by asterisks). SafB1: the first module in saframycin Mx1 synthetase B of Myxococcus xanthus (Genbank Accession No. U24657); GrsA: gramicidin S synthetase A of Bacillus brevis (SWISS PROT Accession No. P14687); HTS1A and HTS1B: the first two modules in HC-toxin synthetase of Cochliobolus carbonum (Q01886); EsynA and EsynB: two modules in enniatin synthetase of Fusarium scirpi (EMBL Accession No. Z18755); ACVA and ACVB: the first two modules in ACV synthetase of Aspergillus nidulans (SWISS PROT P19787); CysnA and CsynB: the first two modules in cyclosporine synthetase of Tolypocladium nivenm (EMBL Accession No. Z28383).

TABLE 6A
A Comparative Amino Acid Sequence Alignment of the Amino-Acid-
Activating Domain (Core 1).

| Consensus | | X L K A G X X X V P I D P X X | | SEQ ID NO:73 |
|---|---|---|---|---|
| | | 10 | | |
| CPS1A | 165 | C F I A G V V A V P I N S V D | (74) | SEQ ID NO:61 |
| CPS1B | 931 | C F V L G A V C I P M A P I D | (74) | SEQ ID NO:62 |
| SafB1 | 96 | C L Y A G V V A V P V Y P P D | (77) | SEQ ID NO:63 |
| GrsA | 109 | V L K A G - G Y V P I D I E Y | (77) | SEQ ID NO:64 |
| HTS1A | 301 | I L K A G G V C V P I D P R Y | (82) | SEQ ID NO:65 |
| HTS1B | 1906 | V V Q A G G V F V L L E P G H | (80) | SEQ ID NO:66 |
| EsynA | 556 | V L K A G H A F T L I D P S D | (63) | SEQ ID NO:67 |
| EsynB | 1626 | I L K A N L A Y L P L D V R S | (65) | SEQ ID NO:68 |
| ACVA | 361 | V W K S G A A Y V P I D P T Y | (76) | SEQ ID NO:69 |
| ACVB | 1455 | V W K S G G A Y V P I D P G Y | (67) | SEQ ID NO:70 |
| CsynA | 556 | I L K A H L A Y L P L D I N V | (70) | SEQ ID NO:71 |
| CsynB | 1642 | I L K A G H A Y L P L D V N V | (68) | SEQ ID NO:72 |

TABLE 6B
A Comparative Amino Acid Sequence Alignment of the Amino-Acid-Activating
Domain (Core 2).

| Consensus | | F T S G X T G X P K G V X X X H R X I | | SEQ ID NO:74 |
|---|---|---|---|---|
| | | 10 | | |
| CPS1A | 253 | F S R A P T G D L R G V V L S H R T I | (312) | SEQ ID NO:75 |
| CPS1B | 1019 | W T Y W - T P D Q R A V Q L G H S Q I | (226) | SEQ ID NO:76 |
| | | * | | |
| SafB1 | 187 | Y T S G S T A D P K G V V L T H R N L | (213) | SEQ ID NO:77 |
| GrsA | 190 | Y T S G T T G N P K G T M L E H K G I | (166) | SEQ ID NO:78 |
| HTS1A | 397 | F T S G S T G V P K C I V V T H S Q I | (154) | SEQ ID NO:79 |
| HTS1B | 2000 | F T S G - T G V P K G A V A T H Q A Y | (166) | SEQ ID NO:80 |
| EsynA | 633 | F T S G S T G I P K G I M I E H R S F | (165) | SEQ ID NO:81 |
| EsynB | 1706 | F T S G S T G K P K G V M I E H R A I | (169) | SEQ ID NO:82 |
| ACVA | 451 | Y T S G T T G F P K G I F K Q H T N V | (172) | SEQ ID NO:83 |
| ACAB | 1538 | Y T S G T T G R P K G V T V E H H G V | (181) | SEQ ID NO:84 |
| CsynA | 640 | F T S G S T G K P K G V M I E H R G I | (172) | SEQ ID NO:85 |
| CsynB | 1724 | F T S G S T G K P K G V M I E H R G V | (174) | SEQ ID NO:86 |

*An insertion (2 residues between R and A) is not shown.

TABLE 6C
A Comparative Amino Acid Sequence Alignment of the Amino-Acid-
Activating Domain (Core 3).

| Consensus | | G E L X V X G X G L A R G Y | | SEQ ID NO:87 |
|---|---|---|---|---|
| | | 10 | | |
| CPS1A | 583 | G E I W V D S P S L S G G F | (32) | SEQ ID NO:88 |
| CPS1B | 1209 | G E I W V Q S E A N A Y S F | (25) | SEQ ID NO:89 |
| SafB1 | 418 | G E I W V R G P S V A Q G Y | (23) | SEQ ID NO:90 |
| GrsA | 374 | G E L C I G G E G L A R G Y | (23) | SEQ ID NO:91 |
| HTS1A | 569 | G E L L I E S G H L A D K Y | (31) | SEQ ID NO:92 |
| HTS1B | 2184 | G E L I I E G S I L C R G Y | (26) | SEQ ID NO:93 |
| EsynA | 816 | G E L V I E S A G I A R D Y | (30) | SEQ ID NO:94 |
| EsynB | 1893 | G E L V V T G D G V G R G Y | (32) | SEQ ID NO:95 |
| ACVA | 640 | G E L H I G G L G I S K G Y | (30) | SEQ ID NO:96 |
| ACVB | 1728 | G E L Y L G G E G V V R G Y | (30) | SEQ ID NO:97 |
| CsynA | 830 | G E L V V S G D G L A R G Y | (23) | SEQ ID NO:98 |
| CsynB | 1916 | G E L V V T G D G L A R G Y | (23) | SEQ ID NO:99 |

TABLE 6D
A Comparative Amino Acid Sequence Alignment of the
Amino-Acid-Activating Domain (Core 4).

| Consensus | | Y - R T G D L X R | | SEQ ID NO:100 |
|---|---|---|---|---|
| CPS1A | 628 | F L R T G L L G F | (13) | SEQ ID NO:101 |
| CPS1B | 1301 | Y V R T G D L G F | (9) | SEQ ID NO:102 |
| SafB1 | 454 | W L R T G D L G F | (11) | SEQ ID NO:103 |
| GrsA | 410 | Y - K T G D Q A R | (8) | SEQ ID NO:104 |
| HTS1A | 609 | Y - R T G D L V R | (8) | SEQ ID NO:105 |
| HTS1B | 2223 | Y - K T G D L V R | (8) | SEQ ID NO:106 |
| EsynA | 860 | Y - R T G D L A C | (9) | SEQ ID NO:107 |
| EsynB | 1939 | Y - R T G D R M R | (10) | SEQ ID NO:108 |
| ACVA | 684 | Y - K T G D L A R | (9) | SEQ ID NO:109 |
| ACVB | 1772 | Y - K T G D L V R | (11) | SEQ ID NO:110 |
| CsynA | 866 | Y - R T G D R A R | (10) | SEQ ID NO:111 |
| CsynB | 1956 | Y - R T G D R A R | (10) | SEQ ID NO:112 |

TABLE 6E
A Comparative Amino Acid Sequence Alignment of the Amino-Acid-Activating
Domain (Core 5).

| Consensus | | L R X D X Q V K I R G X R I E L G E V E | | SEQ ID NO:113 |
|---|---|---|---|---|
| | | 10 20 | | |
| CPS1A | 645 | L G - - L Y E D R I R - Q R V E *N G Q L E | (61) | SEQ ID NO:114 |
| GrsA | 427 | L G R I D N Q V K I R G H R V E L E E V E | (120) | SEQ ID NO:115 |
| HTS1B | 627 | L G R K D T Q V K M N G Q R F E L G E V E | (162) | SEQ ID NO:116 |
| HTS1A | 2248 | V G R S D T Q I K L A G Q R V E L G D V E | (163) | SEQ ID NO:117 |
| EsynA | 878 | L G R M D S Q V K I R G Q R V E L G A V E | (139) | SEQ ID NO:118 |
| EsynB | 1958 | F G R M D N Q F K I R G N R I E A G E V E | (549) | SEQ ID NO:119 |
| ACVA | 702 | L G R A D F Q I K L R G I R I E P G E I E | (123) | SEQ ID NO:120 |
| ACVB | 1792 | L G R N D F Q V K I R G L R I E L G E I E | (116) | SEQ ID NO:121 |
| CsynA | 884 | F G R M D Q Q V K I R G H R I E P A E V E | (149) | SEQ ID NO:122 |
| CsynB | 197 | F G R M D H Q V K V R G H R I E L A E V E | (561) | SEQ ID NO:123 |
| CPS1B | 1397 | L G S I G D T F E V N G L N H F S M D I E | (96) | SEQ ID NO:124 |
| SafB1 | 1662 | S G R R K D L L V I R G R N Y Y P Q D L E | (153) | SEQ ID NO:125 |

*An insertion (two amino acid) between E and N in CPS1A is not shown.
The less conserved cores 5 in CPS1B and SafB1 are indicated by arrows.

TABLE 6F
A Comparative Amino Acid Sequence Alignment of the Thioester
Formation Domain (Core 6).

| Consensus | | F F X X G G D S L X A X X | | | SEQ ID NO:126 |
|---|---|---|---|---|---|
| | | 10 | | | |
| CPS1A | 726 | L D I P F L D S L S E R C | 574 | (193) | SEQ ID NO:127 |
| CPS1B | 1448 | R D P N G Q D S Q M I T E | 530 | | SEQ ID NO:128 |
| SafB1 | 645 | L P D L G L D S L A L V E | 562 | (590) | SEQ ID NO:129 |
| GrsA | 567 | F Y A L G G D S I K A I Q | 471 | | SEQ ID NO:130 |
| HTS1A | 812 | F I H A G G D S I T A M Q | 524 | (1082) | SEQ ID NO:131 |
| HTS1B | 2422 | F F S S G G N S M A A I A | 529 | | SEQ ID NO:132 |
| EsynA | 1040 | F F E M G G N S I I A I K | 497 | (906) | SEQ ID NO:133 |
| EsynB | 2530 | F F Q L G G H S L L A T K | 917** | | SEQ ID NO:134 |
| ACVA | 848 | F F R L G G H S I T C I Q | 500 | (595) | SEQ ID NO:135 |
| ACAB | 1931 | F F S L G G D S L K S T K | 489 | | SEQ ID NO:136 |
| CsynA | 1053 | F F D L G G H S L T A M K | 510 | (577) SEQ ID NO:137 | |
| CsynB | 2551 | F F N V G G H S L L A T K | 922** | | SEQ ID NO:138 |

*Active site for 4′-phosphopantetheine binding.
**Type II modules containing a methyltransferase domain (about 400 amino acids) between cores 5 and 6. All others are type I modules without this insertion.

Amino acid alignment of the two modules of CPS1 to SafB1 indicated that these modules are highly similar to each other in both overall amino acid composition and conserved motif sequences as defined by Stachelhaus and Marahiel (Stachelbaus et al., 1995, supra; Marahiel, 1997, supra). When aligned to other bacterial or fungal peptide synthetases, CPS1 only showed local similarity to cyclosporine synthetase (Weber et al., Current Genetics, 26(2):120 (1994)) and tyrocidine synthetase A (Mootz et al., J. Bacteriol., 179(21):6843 (1997)), but when the amino acids in motif regions were aligned, a overall conservation was observed. Both CPS1A and CPSIB have all five core sequences in the amino-acid-activating domain (Table 6A-E). Cores 3 and 4 are well conserved except for the replacement of an aspartic acid residue of core 4 by a leucine in CPS1A. Cores 1, 2 and 5 show weak conservation, but similar variations are also seen in SafB1. A thiolation domain is found in both modules, which contains a highly conserved motif (core 6, Table 6F). The serine residue in this motif has been shown to be the active site for 4′-phosphopantetheine attachment (Schlumbohm et al., J. Biol. Chem., 266:23135 (1991); Stein et al., FEBS Lett., 340:39 (1994)).

The distances between the six core sequences in the two modules are also largely conserved. Two exceptions are found in the first module, which has 312 amino acids between cores 2 and 3, larger than normal (150-200); 61 between cores 5 and 6, only half of that of most peptide synthetases. SafB1 also shows distance variations at these two interval regions (Table 6B and E). In addition to amino-acid-activating and thiolation domains, CPS1 also has an integrated thioesterase domain (TE) in the carboxy-terminal end of CPS1B (FIG. 12). A signature sequence GXSXG (SEQ ID NO:147), which is highly conserved in animal fatty acid thioesterase type II enzymes and several peptide synthetases, is found in this domain (Table 7).

TABLE 7
Comparative Alignment of Amino Acid Sequences of Active Sites of
Thioesterase Domains (TE) in CPS1 with those of other Peptide
Synthetases.

| Consensus | | X X X X G X S X G X X X A F E X | | SEQ ID NO:139 |
|---|---|---|---|---|
| | | * * * | | |
| | | 10 | | |
| CPS1-TE | 1619 | V L R P G P S S G S E Q H D Q A | (125) | SEQ ID NO:140 |
| ACVA-TE | 3621 | Y H F I G W S F G G T I A M E I | (168) | SEQ ID NO:141 |
| GrsB-TE | 4267 | Y V L I G Y S S G G N L A F E V | (186) | SEQ ID NO:142 |
| GrsT-TE | 1117 | F A F L G H S M G A L I S F E L | (157) | SEQ ID NO:143 |
| SafA-TE | 6313 | L T L F G Y S A G C S L A F E A | (173) | SEQ ID NO:144 |
| TycC-TE | 93 | Y T L M G Y S S G G N L A F E V | (163) | SEQ ID NO:145 |
| TycF-TE | 76 | F A F F G H S M G G L V A F E L | (168) | SEQ ID NO:146 |

ACV:ACV synthetase (SWISS PROT Accession No. P19787); GrsB: gramicidin S synthetase B (P14688); GrsT: the thioesterase encoded by grsT (P14686) in gramicidin S synthetase gene cluster; SrfA: surfactin synthetase A-3 (Q08787); TycC: tyrocidine synthetase C (Genbank Accession No. AF004835); TycF: the thioesterase encoded by tycF (AF004835) in the tyrocidine synthetase gene cluster.
The highly conserved residues (GXSXG; SEQ ID NO:147) are indicated by asterisks.
The number on the left of each amino acid sequence indicates the original position of the first residue; the number on the right (in parentheses) indicates the distance between the last residue shown to the end of each protein.

Sequence homology analysis of TES1 protein. The predicted TES1 protein consists of 367 amino acids (Mᵣ 41013) amino acid alignment of TES1 to hTE, TESB and Mycobacterium tuberculosis TESB homolog (Philipp et al., Proc. Natl. Acad. Sci. USA 93:3132 (1996)) showed that these proteins have an overall 40% identity and 60% similarity. A highly conserved VHS motif (putative active site) is found in the C-terminal region of TES1 at a conserved position (FIG. 13). All these thioesterases have no sequence similarity with the previously identified animal type I or type II thioesterases known to be involved in the chain termination of fatty acid synthesis (Naggert et al., J. Biol. Chem., 266:11044 (1991)). Interestingly, TES1 has more homology to hTE than to two bacterial genes, suggesting that both proteins belong to a new family of eukaryotic thioesterases.

Targeted disruption of CPS1. Disruption of either CPS1A or CPS1B restored the original mutant phenotype. Ten transformants from each of four individual disruption experiments using different constructs, including the plasmid recovered from the REMI insertion site in the mutant (p214B7) and three vectors for chromosome walking (p214SNP, p118BSP and p118BCS) were purified and assayed on N-cytoplasm corn. All transformants showed the same small lesion phenotype as that of the original REMI mutant. Southern blot analysis confirmed that all transformants showing the mutant phenotype resulted from homologous integration of the transforming vector that disrupted the wild type CPS1. No transformants showing the wild type phenotype were obtained, presumably because of the large genomic DNA fragments (over 800 bp in all disruption experiments) on the transforming vector that resulted from high efficiency of homologous recombination and the low chance to recover transformants with ectopic integration.

### EXAMPLE 3

### Targeted Disruption of CPS1 homolog in C. victoriae

Methods and Materials

Strains, growth conditions and transformation. Strains of Cochliobolus species and relatives used for genomic DNA hybridization are listed in Table 8. The strain HyW, a victorin-producing isolate of C. victoriae was recovered from storage and grown on CMX medium (Turgeon et al., Mol. Gen. Genet., 201:450 (1985)) for conidiation or on oat meal agar medium (Churchill et al., Fungal Genet, Newsl. 42A:41 (1995)) for victorin detection at 24° C. under warm white lights (Sylvania Inc., Danvers, Mass.). Transformation was done using the C. heterostrophus procedure (Turgeon et al., Mol. Gen. Gene., 238:270 (1993)).

TABLE 8
Detection of CPS1 homologs in
Cochliobolus spp and relatives

| | | EcoRI | Hybridization | BglII |
|---|---|---|---|---|
| Strain^{a} | Host^{b} | digest^{c} | HindIII digest^{d} | digest^{e} |
| C. heterostrophus | Corn | | | |
| race T (C4) | (Turf-13) | + | 5.2 3.2 | 4.2 |
| race O (C5) | | + | 5.2 3.2 | 4.2 |
| C. carbonum | Corn¹ | | | |
| race 1 (26R13) | (hm1hm1) | + | 6.6 | 5.0 |
| race 2 (YugY) | | N | 6.6 | 5.0 |
| race 3 (BZ1703)* | | N | 6.6 | 5.0 |
| C. victoriae (HvW) | Oats (Vb) | + | N | 5.0 |
| C. sativus (A20) | Grasses² | + | 3.0 | N |
| C. specifer (D5-7) | Grasses² | + | N | N |
| C. homomorphus | Unknown | N | 5.8 | N |
| (ATCC 13409) | | | | |
| C. dactyloctenii | Unknown | N | 5.9 | N |
| (7938-9) | | | | |
| S. turcica (NK2) | Sorghum and | + | N | N |
| | maize³ | | | |
| S. rostrata (32197) | Weeds and | + | 2.8 | N |
| | bamboo⁴ | | | |
| B. sacchari | Sugarcane⁵ | | | |
| (764-1) | | + | 5.4 2.5 | N |
| (1249-10) | | N | 5.4 2.5 | N |
| ^{a.}C. = Cochliobolus. S. = Setosphaeria. B. = Bioplaris. The name of isolates (or lab strains) of each species are given in parentheses and those known to produce host-specific toxins are underlined. | | | | |
| *Provided by Tsukiboshi Takao (Japan) and the isolate could be either BZ1209 or BZ1703. | | | | |
| ^{b.}Genotype susceptible to the host-specific toxin-producing isolate is given in parentheses. References for hosts of those species not mentioned are as follows: 1: Welz et al., Phytopathology, 83: 593 (1993); Leonard et al., Phytopathology, 80: 1154 (1990) (for races 2 and 3 only). 2: Domsch et al., “Compendium of Soil Fungi, Vol. 1,” New York, New York: Academic Press, pp 216-222 (1980). 3: David et al., “Fungi on | | | | |
| #Plants and Plant Products,” St. Paul, Minnesota: APS Press, p. 635 (1989); Thakur et al., Plant Dis.,73: 151 (1989). 4: Rao et al., Indian Bot. Rep.,6: 38 (1987); Bhat et al., Curr. SCI. (BANGALORE), 58: 1148 (1989). 5: Yoder, Ann. Rev. Phytopathol., 18: 103 (1980). | | | | |

^{c.}Genomic DNAs (from a previously prepared gel blot filter, Rose et al., 1996, supra) were probed with the 3.4 kb CPS1 fragment cloned on p214B7. “+” indicates a strong hybridization signal. All species hybridized to a large fragment (about 23 kb).
^{d.}Genomic DNAs selected from a collection were probed with the CPS1 3.2 kb fragment cloned on p214S1. The size of fragments that hybridized to the probe is given in kb. The intensities of hybridization signals were similar to each other. N = not done.
^{e.}Genomic DNAs were probed with the same CPS1 fragment as in c.

DNA manipulations and targeted disruption of the CPS1 homolog of C. victoriae. Genomic DNAs for probing were prepared according to Yoder, In: Genetics of Plant Pathogenic Fungi, Vol. 6, San Diego, Calif.:Academic Press, Sidhu, ed., pp. 93-112 (1988)), or selected from a lab DNA collection (stored at 4° C.). A gel blot filter bearing known genomic DNAs was also probed. Plasmid DNA preparation, restriction enzyme digestions, gel electrophoresis, gel blot analysis were done using standard protocols (Sambrook et al., 1989, supra). For probing, CPS1 fragments of C. heterostrophus cloned on p214B7 (3.4 kb left flank) and p214S 1 (3.2 kb right flank) were prepared by restriction enzyme digestion of the plasmid DNAs followed by purification using the QIAquick Gel Extraction Kit (QIAGEN Inc., Chatsworth, Calif.). The plasmid p18B14, which carries the 2.3 kb BglII fragment of CPS1 interrupted by the hygB cassette was linearized with BglII and introduced into HvW genome. Transformants were purified by isolation of single conidia and genomic DNAs were digested with BglII and probed with the CPS1 3.2 kb fragment.

Bioassays. Pathogenicity was determined by an oat plant assay. Fungal strains were grown in individual oat meal agar medium plates (60×15 mm) containing hygromycin B (60 μg/ml) for 10 days at 24° C. under lights. Conidia were scraped from the plates and suspended in 6 ml sterilized distilled water. One ml of conidial suspension of each strain was mixed with 60 seeds of susceptible or resistant oats. Inoculated seeds were planted in 4″×6″ pots and seedlings were allowed to grow for two weeks. Seed germination rate and symptom development were recorded at different stages (4, 6, 8 and 24 days after inoculation). Detection of victorin production using HPLC analysis was done by Alice Churchill in Dr. Vladimir Macko's lab at Boyce Thompson Institute for Plant Research.

Results

Detection of CPS1 homologs. Genomic DNAs of 12 isolates (or lab strains) of 9 fungal species hybridized to CPS1 (Table 8). All 6 Cochliobolus species, including 4 known plant pathogens (C. carbonum. C. victoriae, C. sativus and C. specifer) and 2 species with unknown hosts (C. homomorphus and C. dactyloctenii) gave hybridization signals of the same intensity as that of C. heterostrophus CPS1 fragments. Two phytopathogenic Setosphaeria species and Bioplaris sacchari, a sugarcane pathogen gave a similar hybridization intensity.

CPS1 homologs appear to be polymorphic among different species, i.e., all species gave one or two unique bands when BglII or HindIII digested genomic DNAs were probed (except for C. victoriae, which showed the same hybridization pattern as C. carbonum) (Table 8). Interestingly, EcoRI digested genomic DNAs of the same species did not show polymorphisms; all species hybridized to a large fragment (about 23 kb, Table 8), indicating the absence of an EcoRI site in all CPS1 homologs as in the C. heterostrophus gene. In C. hererostrophus, a >12 kb of genomic region which includes CPS1 (5.4 kb), TES1 (1.1 kb) and sequence downstream of the 3′ end of CPS1 has no EcoRI sites. In contrast to species-dependent polymorphisms, CPS1 homologs appear to be highly conserved among different isolates of the same species. Both C. heterostrophus race T and race O hybridized to the same 4.2 kb BglII fragment (or 5.2 and 3.2 kb HindIII fragments); all three C. carbonum races hybridized to the same 5.0 kb BglII fragment (or 6.6 kb HindIII fragment) (Table 8) and B. sacchari isolates 764-1 and 1249-10 hybridized to the same HindIII fragments (5.4 and 2.5 kb) (Table 8).

Twenty tansformants were obtained from transformation of the victorin-producing isolate HvW with BglII-linearized plasmid p118B14. Six transformants were purified and assayed for both victorin production and pathogenicity to susceptible oat plants. All transformants produced wild type levels of victorin as determined by HPLC analysis, but four of them (Tx7, Tx2, Tx5 and Tx8) showed dramatically reduced virulence in the plant assay. The seed germination rate on the eighth day after inoculation is only 13-25% for wild type and two transformants (Tx9 and Tx4), but 45-63% for the other four transformants. One day 24 after inoculation, all plants emerged from the seeds inoculated with wild type, Tx9 or Tx4 were killed but most (29-63%) from the seeds inoculated with Tx2, Tx7, Tx5 or Tx8 still survived (Table 9). Southern blot analysis confirmed that transformants showing the reduced virulence phenotype resulted from homologous integration of the transforming vector that disrupted the wild type CPS1 homolog in C. victoriae genome; transformants showing the wild type phenotype resulted from ectopic integration events that left the native gene intact. All transformants remained nonpathogenic to resistant oats, indicating that disruption of the CPS1 homolog does not affect host specificity of the fungus.

TABLE 9
Disease development of oat plants inoculated with C. victoriae
transformants (Tx).

| | No. germinated^{b} | | | Germination Rate | No. survivors^{d} | |
|---|---|---|---|---|---|---|
| Strain^{a} | 4 | 6 | 8 | (%)^{c} | 24 | % |
| Control-1 | 28 | 41 | 45 | 75 | 75 | 100 |
| Control-2 | 40 | 50 | 50 | 83 | 50 | 100 |
| Control-3 | 1 | 7 | 12 | 20 | 0 | 0 |
| Tx2 | 8 | 26 | 27 | 45 | 16 | 59 |
| Tx4 | 5 | 15 | 15 | 25 | 0 | 0 |
| Tx5 | 2 | 24 | 28 | 47 | 8 | 29 |
| Tx7 | 14 | 36 | 38 | 63 | 24 | 63 |
| Tx8 | 7 | 29 | 29 | 47 | 13 | 47 |
| Tx9 | 0 | 3 | 8 | 13 | 0 | 0 |

^{a}Control-1 = uninoculated susceptible oat seeds. Control-2 and Control-3 = resistant and susceptible oat seeds inoculated with wild type C. victoriae (isolate HvW), respectively. Six transformants were tested on both resistant and susceptible seeds, but only data for the later are shown (all transformants gave the same results as Control-2 when tested on resistant seeds). Repeat experiments gave similar results (data not shown).
^{b}Sixty oat seeds were used for each strain. Emerged oat plants were counted 4, 6 and 8 days after inoculation.
^{c}Calculation based on the data collected on the day 8.
^{d}Recorded on day 24 after inoculation. The percentage of survivors is based on the number of plants recorded on days 8 and 24.

Discussion

CPS1 encodes an enzyme with an adenylation domain. A gene designated CPS1 was cloned from the corn pathogen C. heterostrophus using the REM1 mutagenesis procedure. Structural and functional analyses strongly suggest that CPS1 encodes an enzyme with one or more adenylation domains, e.g., a CoA ligase. CPS1 contains two repeated functional units with a modular organization, and has a thioesterase motif (GXSXG; SEQ ID NO:147). This motif has been demonstrated to be an active site for catalyzing release of medium-chain-length (C₈₋₁₂) fatty acids in fatty acid synthases and potentially for termination of peptide chains or for repeated acyl transfer reactions because the same motif is also the characteristic of acyl transferases or acyl transfer domains (AT) of fatty acid synthases (FAS) and polyketide synthases (PKS) (Krätzschmar et al., J. Bacteriol., 171, 5422, (1989)).

Although similar TE domains are found in certain fungal PKSs, i.e., Aspergillus nidulans pksL1 gene (Feng and Leonard, J. Bacteriol, 177, 6246, (1995)) and pksST gene (Yu and Leonard, J. Bacteriol., 117, 4792, (1995)), CPS1 is unlikely to be a polyketide synthase because: 1) it does not show any significant similarity to known PKSs, and 2) it lacks unique functional domains found in these proteins such as the ketoacyl synthase domain (KS) and the acyl transferase domains (AT) found in the N-terminal region of all fungal PKSs (Yang et al., 1996, supra). This does not exclude the possible common evolutionary origin of CPS1 and PKSs (Stachehaus and Marahiel, 1995, supra).

CPS1 could be responsible for biosynthesis of an unidentified peptide phytotoxin. It is well known that several Cochliobolus species and related filamentous fungi produce peptide toxins. These include C. carbonum and C. victoriae, two species most closely related to C. heterostrophus. The former produces HC-toxin as mentioned above; the latter produces victorin, a chlorinated cyclized peptide. Alternaria alternata, a plant pathogenic species from a genus closely related to Cochliobolus, is also known to produce several peptide toxins such as AM-toxin, a cyclic tetradepsipeptide produced by A. alternata apple pathotype and tentoxin, a cyclic tetrapeptide produced by A. alternata pv. tenuis (Nishmura and Kohmoto, 1983). These findings have lead to the postulation that, in addition to T-toxin, C. heterostrophus might also produce a similar secondary metabolite, such as a hypothetical “race O” toxin (Yoder, 1981).

Interestingly, a Tox⁺, cps1⁻ mutant showed reduced virulence on T-cytoplasm corn although it produced the same amount of T-toxin as wild type race T. This is unusual because the interaction between T-toxin and the T-corn-unique URF13 protein is highly specific; the same outcomes should be expected if two strains that produce the same amount of T-toxin attack the same host, T25 corn. The most likely explanation for this result is that the fungal growth in planta has been inhibited by the host plant and the poor growth results in reduced T-toxin production which is normal when the fungus is grown in culture. Reduced virulence on T-cytoplasm corn is due to the reduced T-toxin production as that seen in leaky Tox⁻ mutants. This inhibition of growth could be due to the failure of suppression of the host defense mechanism by the fungus, which is mediated by the CPS1 controlled peptide toxin. A cps1⁻ mutant that fails to produce this “suppresser” could not be able to colonize plant tissues as vigorously as wild type does, resulting in the reduced ability to cause disease as indicated by the smaller lesion phenotype. If this turns out to be the case, CPS1 should be considered as a general virulence factor as proposed for enniatin.

It is possible that cps1⁻ mutants are still be able to produce a certain amount of CPS1 toxin. One probability is the gene has not been completely inactivated by insertional mutagenesis or targeted disruption. The original REMI insertion occurred at core sequence 1 of CPS1A, a region that might be not critical (function of core 1 is unknown). The second targeted site is located between cores 1 and 2 of CPS1B and the third is located between cores 2 and 3 of the same module. All three insertions do not disrupt critical motifs. On the other hand, CPS1 contains a number of in-frame start codons and some of them are located immediately downstream of these insertion sites. It is possible that each of these disruptions actually resulted in two subtranscripts, one is transcribed normally from the start codon of CPS1 and stops at the insertion site and second is transcribed near one of these in-frame ATGs downstream of the insertion site and stops at the end of CPS1. Both transcripts could give a truncated protein that still has enzymatic activities. But these separate enzymes might have affinities for their substrates lower than that of holoenzyme. The reduced production of CPS1 toxin might be due to the CPS1 holoenzyme having been split into two fractions by the vector insertion and the resulting truncated proteins being much less active than the original polypeptide. This hypothesis can be tested by construction a C. heterostrophus strain in which the entire CPS1 encoding sequence has been deleted.

The second possibility is the existence of multiple copies of CPS1 in the genome. Previous studies have demonstrated that the gene encoding HC-toxin synthetase (HTS1) is duplicated in the genome and both copies (HTS1-1 and HTS1-2) are 270 kb apart in most Tox2+isolates of C. carbonum (Ahn and Walton, 1996, supra). Disruption of either copy reduced HTS1 activity but did not affect HC-toxin production; when both copies were disrupted, HC-toxin production was abolished (Panaccione et al, 1992, supra). But in contrast to the case of HTS1, gel blot analysis does not indicate the presence of a second copy of CPS1 and disruption of CPS1 does affect the production of the putative toxin. It is unlikely that two genes with similar organization are in the genome. An alternative postulation is that there may be a second gene which encodes a protein with the same enzyme activity as CPS1 but does not have significant sequence homology to CPS1. This hypothesis is hard to test unless this gene is clustered with CPS1 and can be recovered by chromosome walking.

In conclusion, pathogenesis by C. heterostrophus to corn involves at least two secondary metabolites: the T-toxin, a host specific factor which determines high virulence on a particular host, T-corn and the hypothetical CPS1 toxin, a general factor (either virulence or pathogenicity factor) which contributes to basic mechanisms underlying the disease establishment by the fungus in common host plants.

### EXAMPLE 4

### CPS1 Orthologs

As described above, Cochliobolus heterostrophus gene CPS1 encodes a putative peptide synthetase that appears to be a general factor for fungal virulence to its hosts. CPS1 has been found to be highly conserved among at least 9 fungal species belonging to 3 genera including the genus Cochliobolus and closely related genera Bioplaris and Setosphaeria; it has been demonstrated to be required for pathogenesis by three different plant pathogens, i.e., C. heterostrophus race O, race T to corn and C. victoriae to oats (Lu, 1998, Ph.D. thesis, Cornell University).

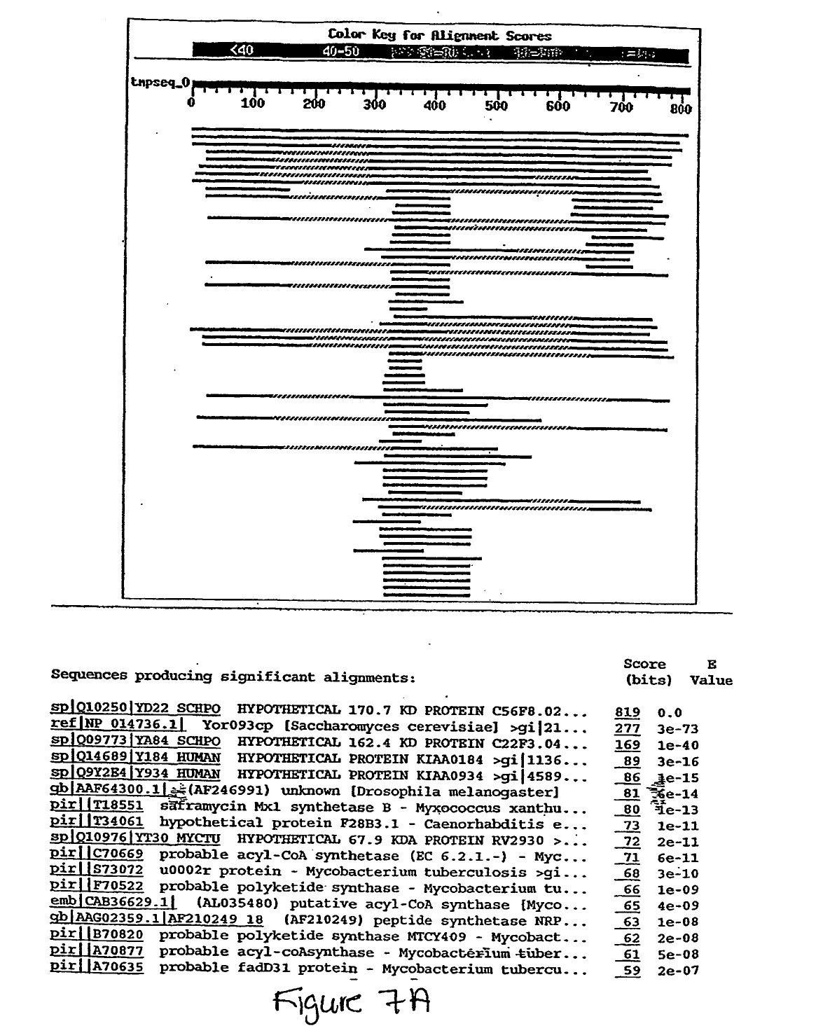

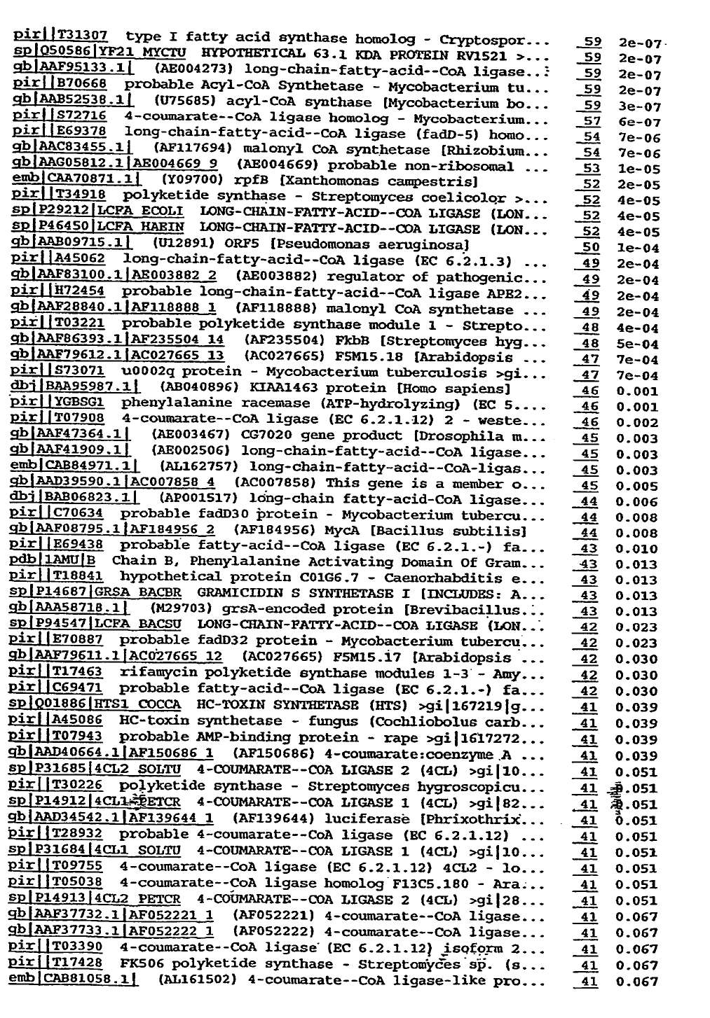

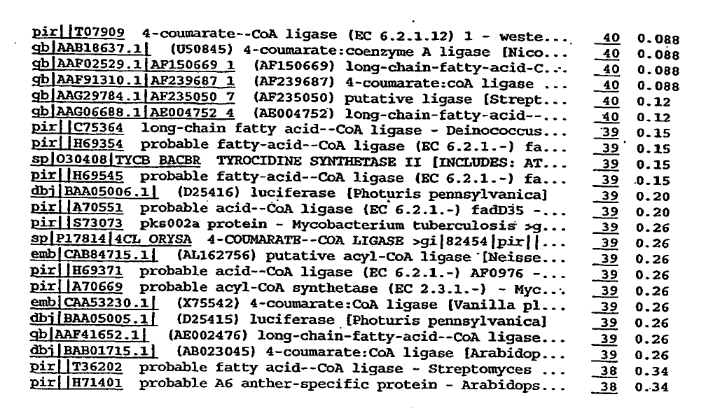

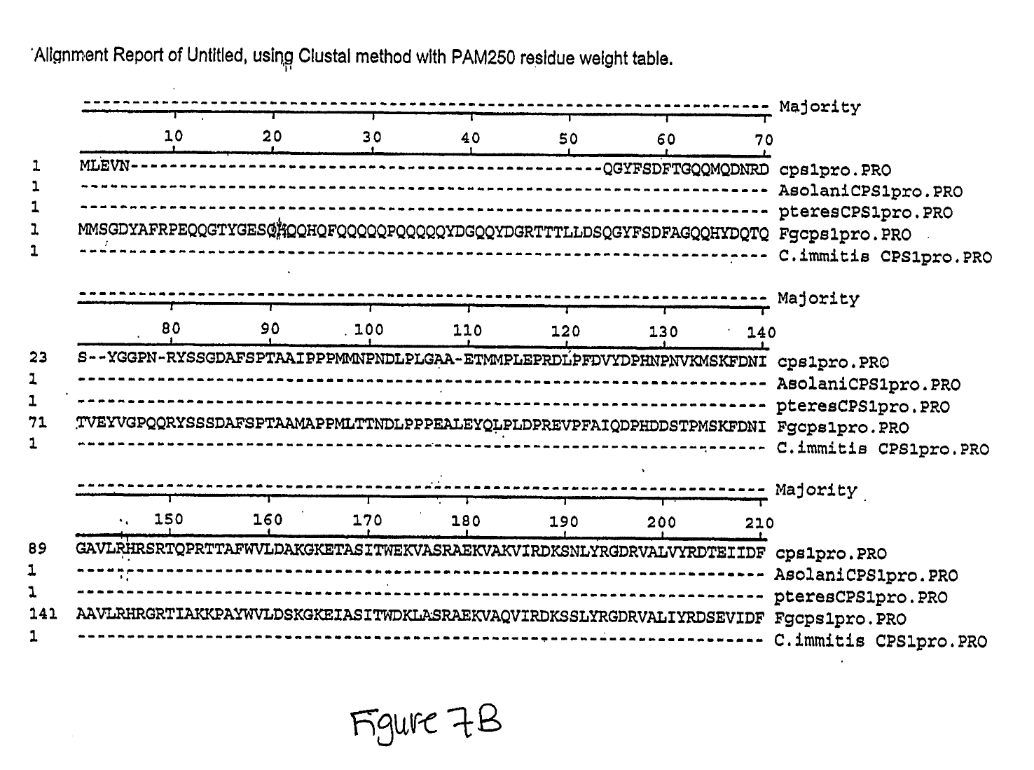

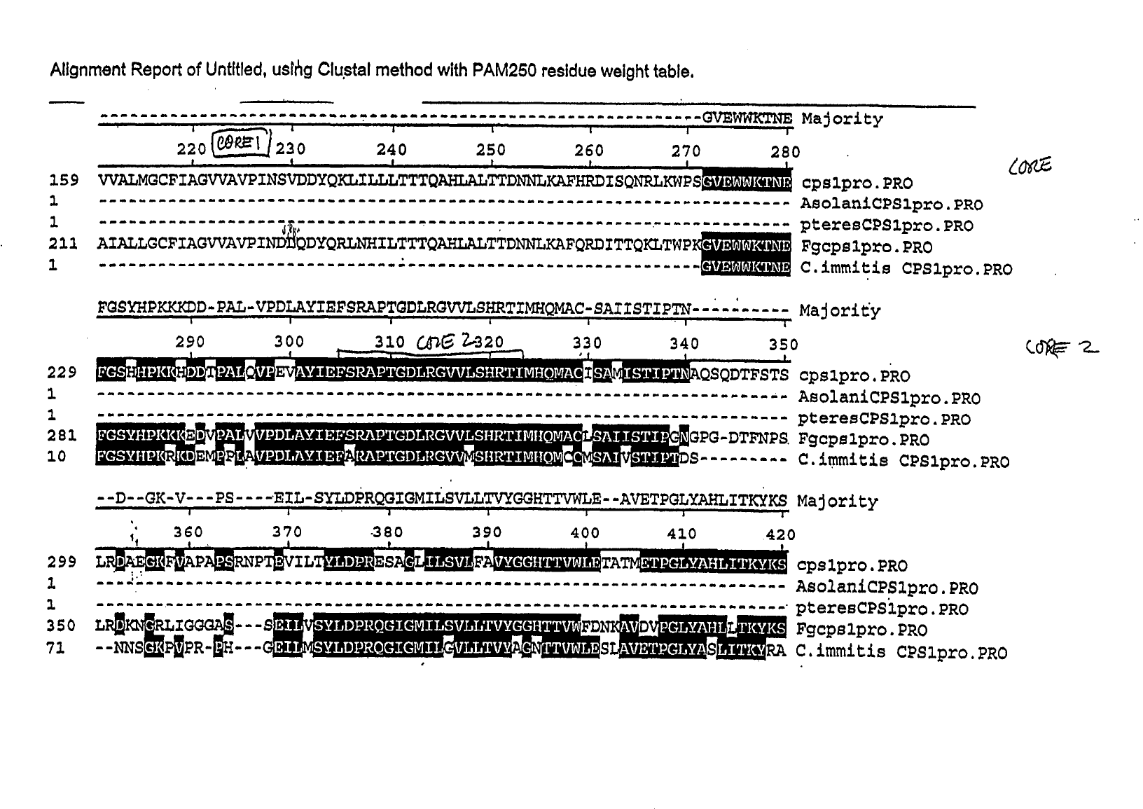

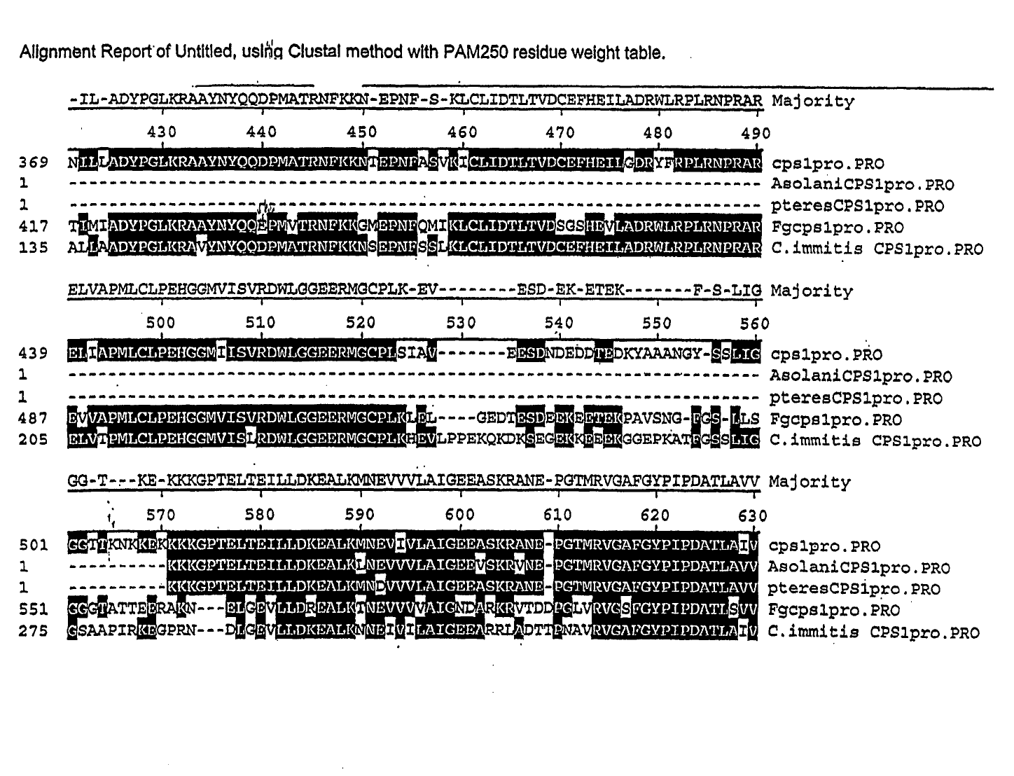

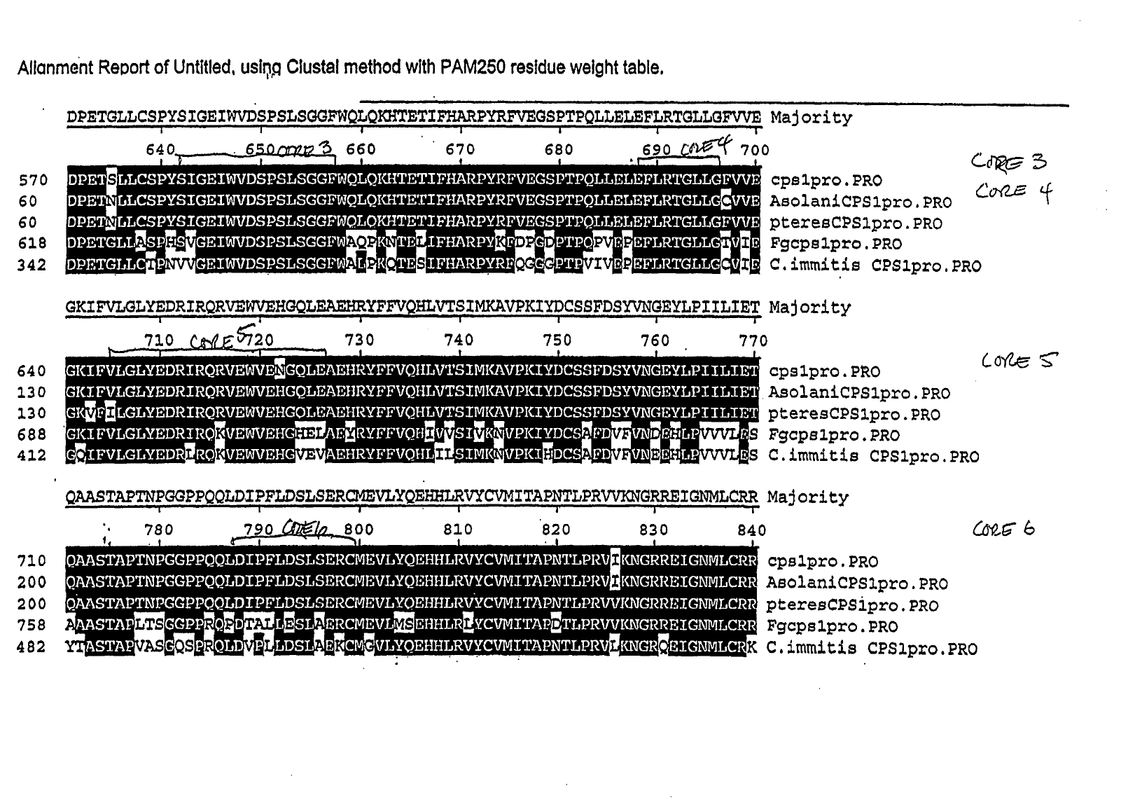

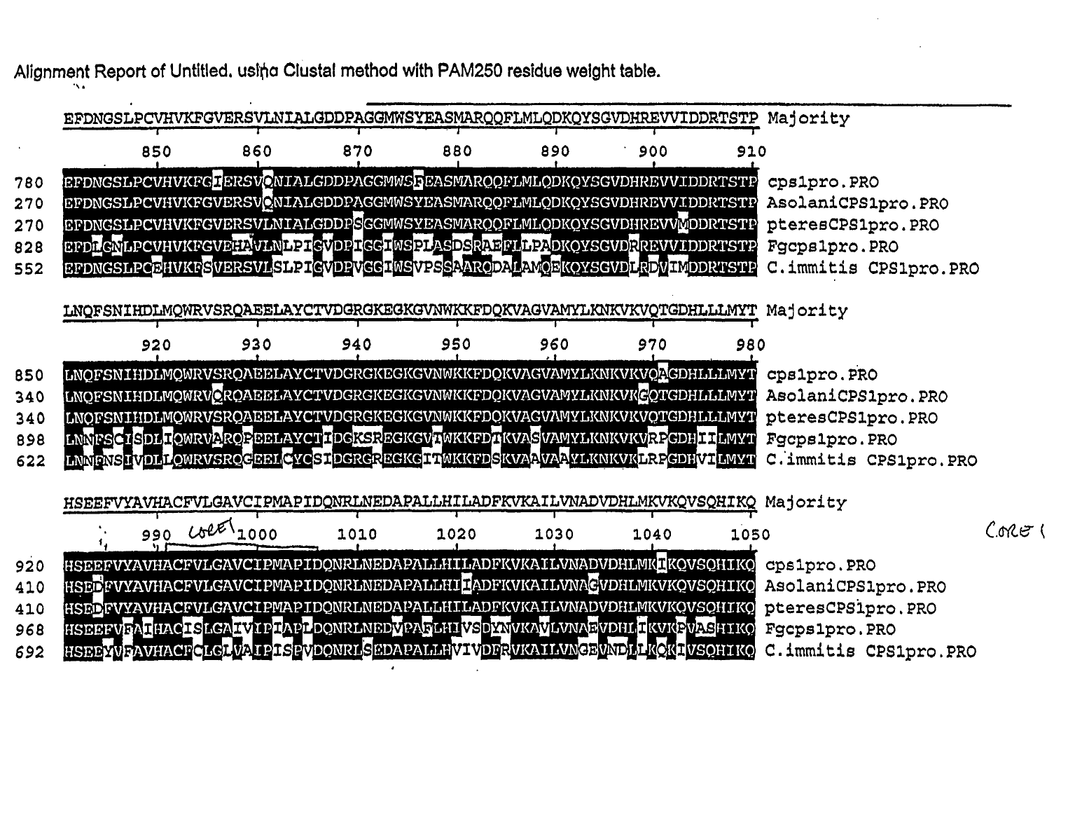

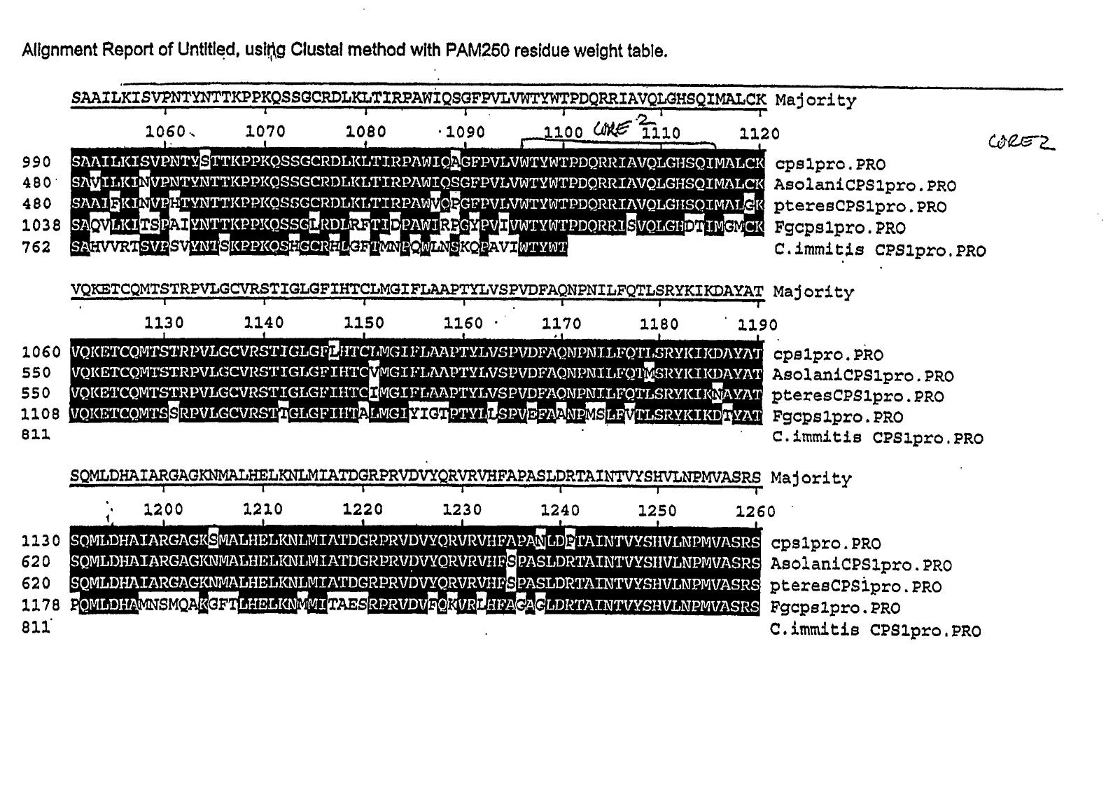

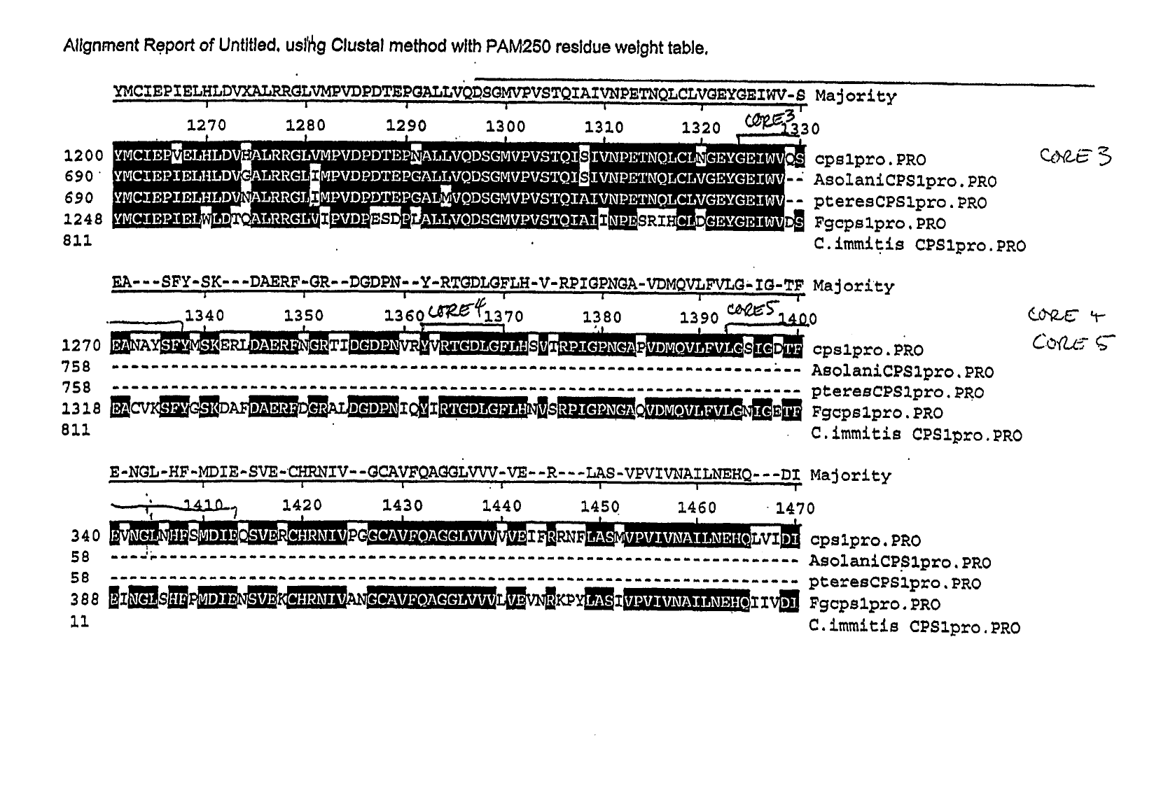

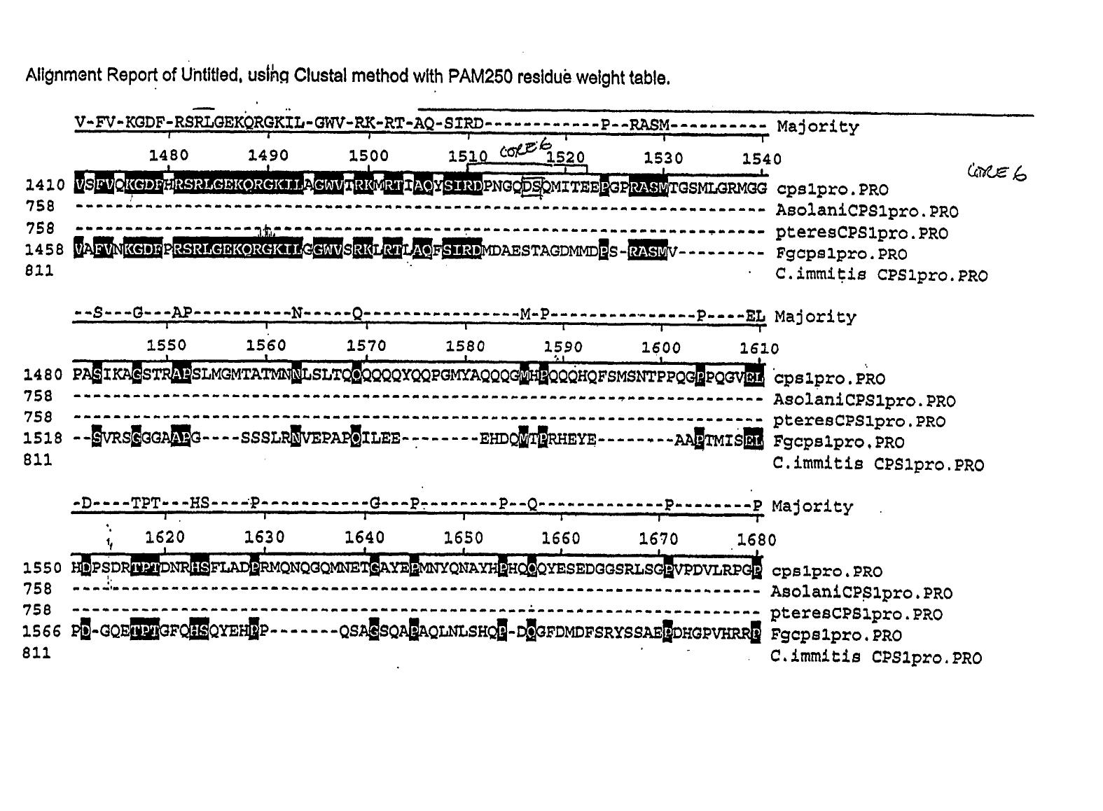

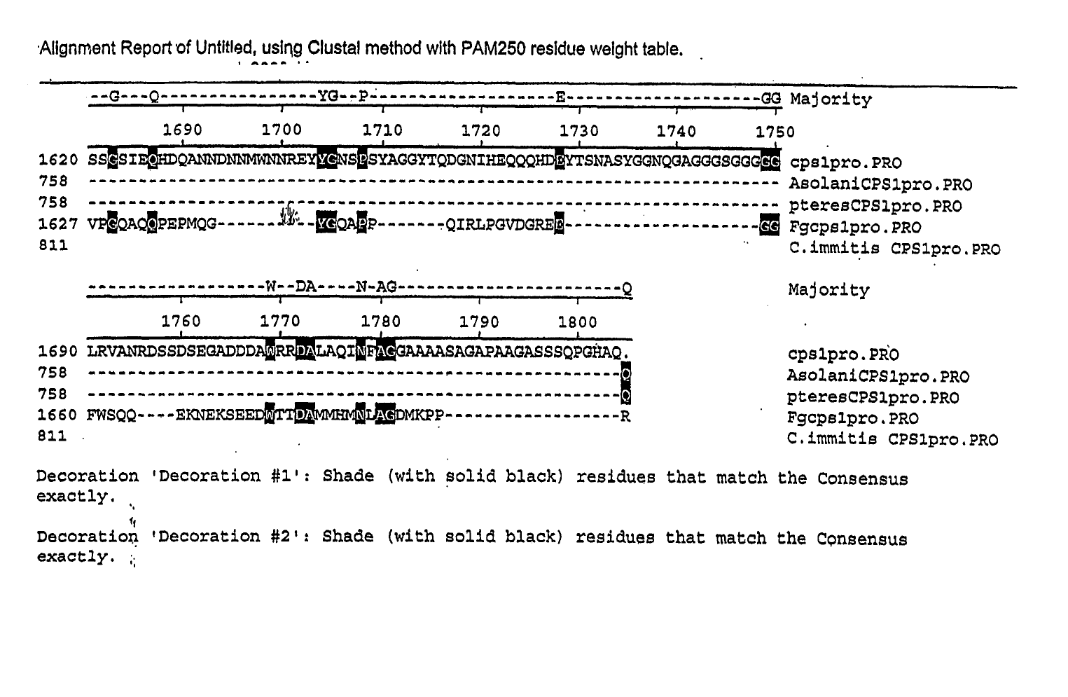

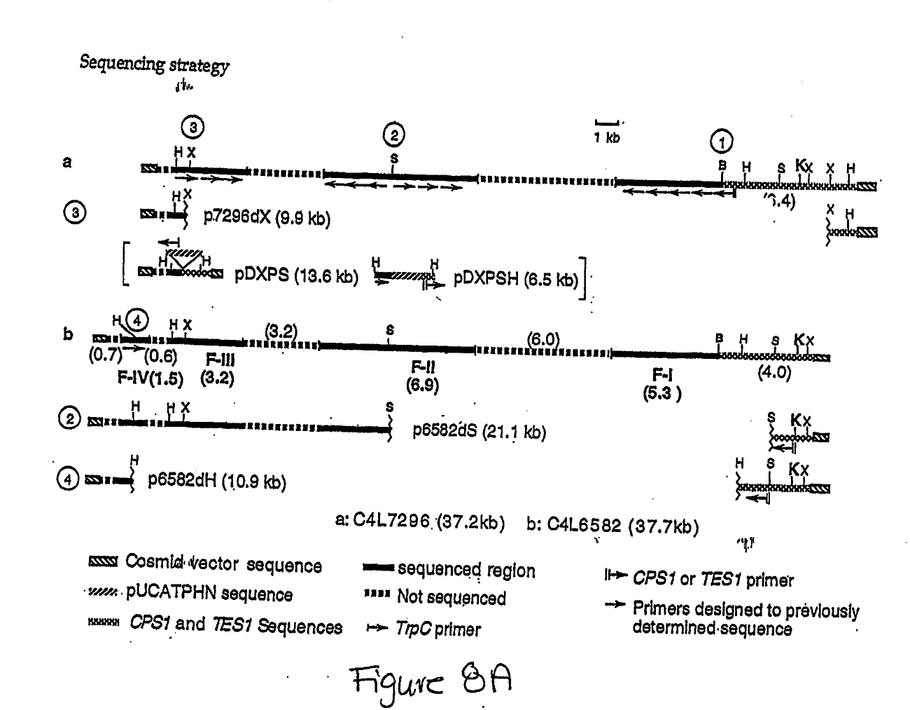

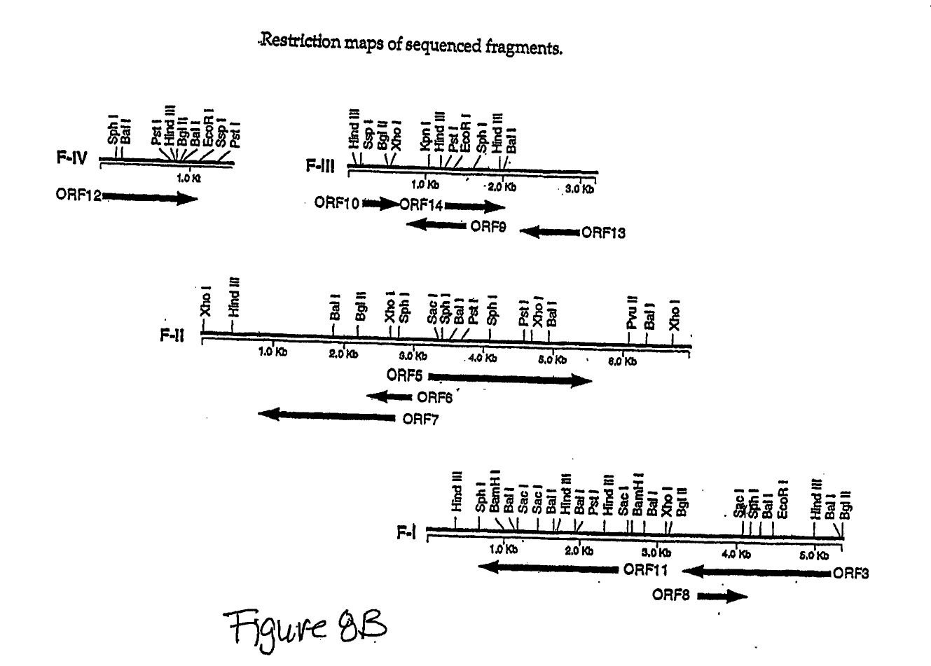

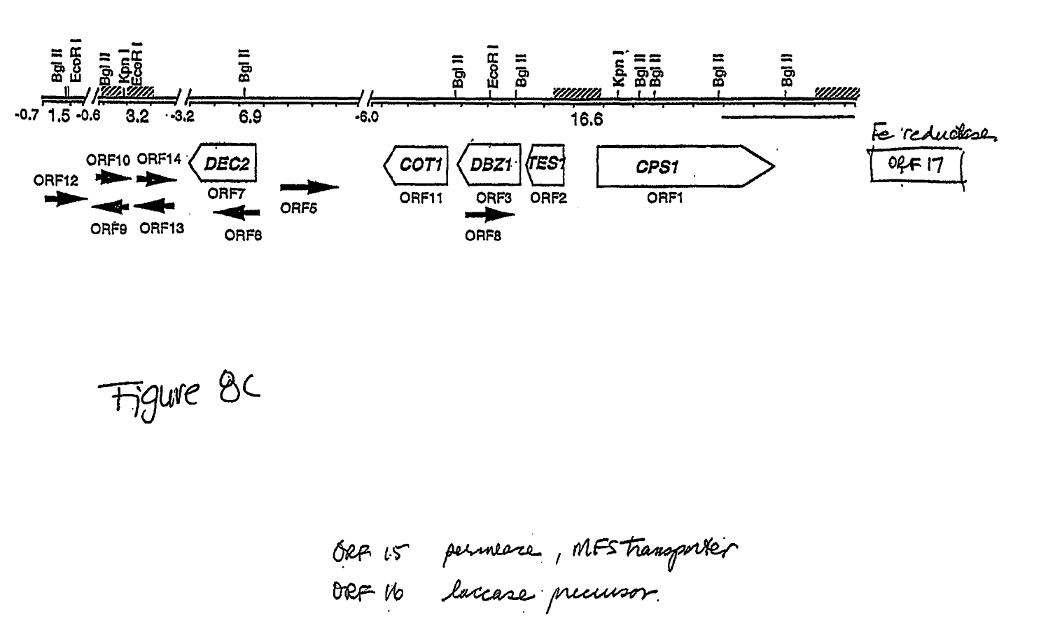

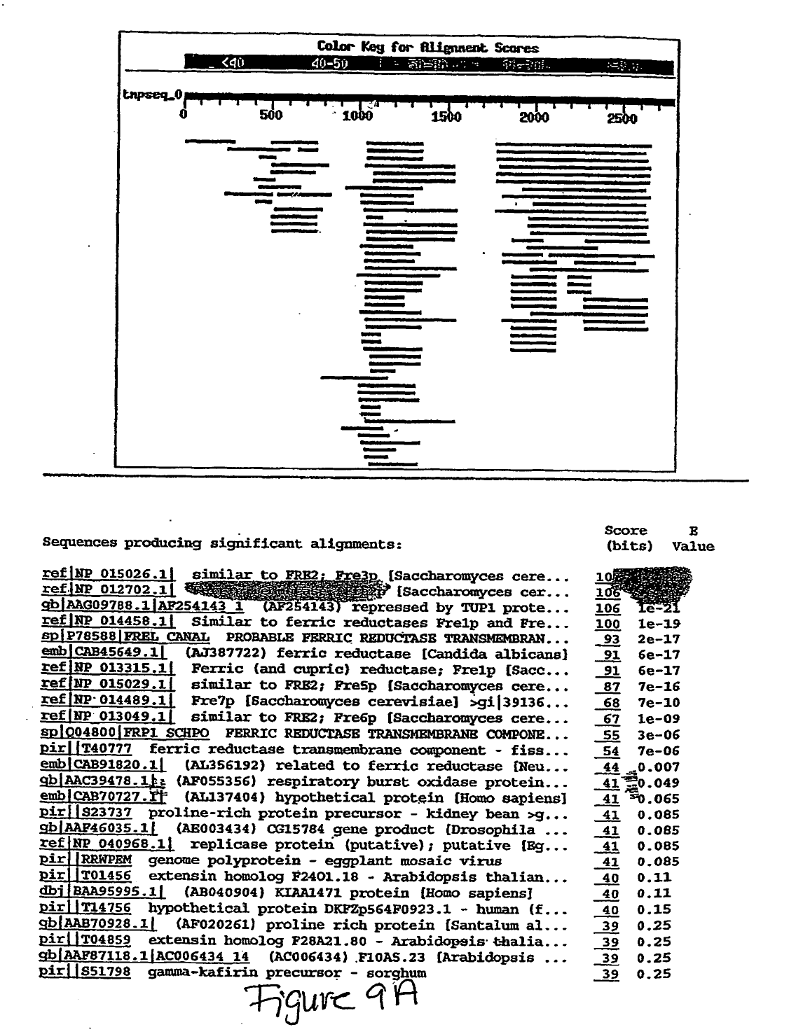

To further explore the role of CPS1 in fungal pathogenesis and its conservation in other fungi, genomic DNAs of additional species of Cochliobolus and other closely or distantly related genera were probed with ChCPS1 by DNA-DNA hybridization (Lu, S.-W., B. G. Turgeon and O. C. Yoder. 1999. Fungal Genetics Conference, March 1999, Pacific Grove, Calif.). Genomic DNAs of 40 field isolates (or lab strains) representing 34 fungal species belonging to 16 genera hybridized when probed with ChCPS1 (FIG. 4). All 16 Cochliobolus species, including the known plant pathogens C. carbonum, C. victoriae, C. miyabeanus, C. sativus and C. specifer, and five genera closely related to Cochliobolus, i.e., Pyrenophora, Setosphaeria, Bipolaris, Stemphylium and Alternaria showed hybridization intensities comparable to that of C. heterostrophus itself (FIG. 4A). DNAs of species from nine distinctly related genera, including several of economic importance (e.g., Magnaporthe grisea, Fusarium graminearum, Gaeumannomyces graminis) or of medical importance (e.g., Candida albicans) hybridized weakly to CPS1 (FIGS. 4B and 4C) whereas no signal was detected in DNA of the basidiomycete Ustilago maydis.

Homologs of CPS1 were further identified by polymerase chain reaction (PCR) using degenerate primers designed to conserved regions of C. heterostrophus CPS1 (ChCPS1). Four CPS1 homologs were cloned and characterized. Three of them were cloned from phytopathogenic fungi, including the wheat head scab fungus Fusarium graminearum (FgCPS1, 6003 bp, SEQ ID NO:40), the potato early blight fungus Alternaria solani, (AsCPS1, 2369 bp, SEQ ID NO:42) and the barley net blotch fungus Pyrenophora teres (PtCPS1, 2320 bp, SEQ ID NO:44). The fourth was cloned from the human pathogenic fungus Coccidioides immitis (CiCPS1, 2435 bp SEQ ID NO:46). The complete FgCPS1 gene was cloned using both PCR amplification and plasmid rescue procedures preceded by targeted gene disruption of this gene in the genome. The remaining three CPS1 homologs were partially cloned by direct PCR amplification.

The FgCPS1 open reading frame (5125 bp) has 50% nucleotide identity to ChCPS1 in about 4.4 kbp of overlap. No “TATA” box-like element was found in the 5′ untranslated region, but other promoter sequences including two putative “CAAT” boxes and a “CT” motif were located upstrearm of the start codon (ATG). There is only one putative intron found 1508 bp upstream of the stop codon (TGA) in contrast to three in ChCPS1.

A putative polyadenylation signal “AATAA” is located 62 bp downstream of the stop codon. The predicted FgCPS1 protein (1692 amino acids, Mᵣ 187983 Da, SEQ ID NO:41) has 68% identity, 73% similarity to ChCPS1 in about a 1,500 amino acid overlap that contains two structurally similar modules highly similar to those of ChCPS1 (FIG. 7B). FgCPS1 has no significant similarity to ChCPS1 at the C-terminus, which is shorter and lacks the thioesterase domain seen in ChCPS1.

AsCPS1 (2369 bp, SEQ ID NO:42) has 76% nucleotide identity to ChCPS1 in the entire cloned region which contains two conserved introns. The translated AsCPS1 protein (partial) includes 758 amino acids (SEQ ID NO:43) corresponding to amino acids 511-1269 in ChCPS1 and has up to 93% identity, 95% similarity to ChCPS1 (FIG. 7B).

PtCPS1 (2320 bp, SEQ ID NO:44) has 78% nucleotide identity to ChCPS1 in the entire cloned region which contains only one intron. The translated PtCPS1 protein (partial) includes 758 amino acids (SEQ ID NO:45) corresponding to amino acids 511-1269 in ChCPS1 and has 93% identity, 96% similarity to ChCPS1.

CiCPS1 (2435 bp, SEQ ID NO:46) has 65% nucleotide identity to ChCPS1 in the entire cloned region which has no introns. The translated CiCPS1 protein (partial) includes 812 amino acids (SEQ ID NO:47) corresponding to amino acids 511-1040 in ChCPS1 and has 67% identity, 80% similarity to ChCPS1 (FIG. 7B). Another ortholog in Candida was identified by Southern blot (see FIG. 4).

BLAST searches using SEQ ID NO:41 (FIG. 6) and SEQ ID NO:47 (FIG. 7A) identified orthologs of those fungal CPS1s.

Disruption of FsCPS1 in F. graminearum (=Gibberella zeae), the wheat head scab fungus, caused significantly reduced virulence to wheat. All cps 1⁻ disruptants of F. graminearum showed at least 50% (when inoculated with 10⁵/ml condidia) or even 80-90% (when inoculated with 10⁴/ml condidia) reduction in ability to cause a typical “white head” symptom on the host whereas in the same conditions, ectopic transformants caused disease symptoms indistinguishable from wild type. These results suggest that CPS1 is also required for pathogenesis by fungi that are distantly related to C. heterostrophus, arguing that these peptide synthetase gene homologs might control biosynthesis of a general fungal virulence factor.

Discussion

Conservation of CPS1 and taxonomy. By genomic DNA hybridization, C. heterostrophus CPS1 homologs were found in 16 additional fungal species belonging to 5 genera Hybridization signals for some were as strong as the C. heterostrophus gene, indicating that CPS1 is highly conserved among these fungi. This conservation appears to match the taxonomic relationships between these species. Cochliobolus (anamorph Bipolaris) and Setosphaeria (anamorph Exserohilum) are closely related genera.

Two species, C. victoriae and C. carbonum, which are able to cross to each other and thus may not be different species (Scheffer et al., 1967; Yoder et al., 1989), showed the same hybridization pattern to CPS1. B. sacchari, the closest asexual relative of C. heterostrophus, hybridized to two HindIII fragments that were only seen in C. heterostrophus itself, but all other species gave only one distinct polymorphic band. Phylogenetic analyses using the internal transcribed spacer (ITS) sequences and fragments of the GPD (vanWert and Yoder, 1992) and MAT genes (Turgeon et al., 1993, supra) also put C. victoriae/C. carbonum and C. heterostrophus/B. sacchari closest to each other (Turgeon and Berbee, 1997). These results might imply that CPS1 has coevolved with these genes.

CPS1 homologs and pathogenesis. The genera Cochliobolus and Setosphaeria include many plant pathogenic species that are commonly associated with leaf spots or blights, mainly on cultivated cereals and wild grasses (Sivanesan, 1987; Alcorn, 1988). This group of phytopathogenic fungi includes both mild pathogens and severe pathogens that often produce host-specific toxins (Yoder, 1980, supra). One of the essential questions is whether or not the various diseases on diverse host plants caused by these fungi involve common factors or depend only on individual specific factors, such as host-specific toxins.

Previous studies have shown that host-specific toxins can be critical factors for determining either virulence or host-range, but they do not account for general pathogenicity since they are produced only by certain isolates in the species and the corresponding biosynthetic genes are found only in these toxin-producing isolates (Yoder et al., 1997, supra). In contrast, CPS1 homologs are found in all Cochliobolus and Setosphaeria species tested so far, suggesting they are a common factor shared by this group. Disruption of the CPS1 homolog in the oat pathogen C. victoriae caused dramatically reduced virulence to victorin-susceptible oats although the transformants produced wild type levels of victorin. This result is similar to that with C. heterostrophus race T, in which cps I⁻ disruptants still produced wild type levels of T-toxin but showed reduced virulence on T-cytoplasm corn. These results argue strongly that host-specific toxins alone are not sufficient in determining the ultimate outcome of fungus/plant interactions and suggest that the establishment of disease by these fungi also requires CPS1, which might control a pathway for general pathogenicity.

The CPS1 gene cluster and homologs could be fungal “pathogenicity islands”. In the early 1990s, studies on pathogenesis by uropathogenic E. coli led to the identification of pathogenicity gene clusters, termed “pathogenicity islands” (Hecker et al., 1990; Blum et al., 1994). Subsequently, similar gene clusters were identified in additional animal or human bacterial pathogens, including Yersinia pestis, Helicobacter pylori and Salmonella typhimurium. These islands often contain genes for production of toxins or genes encoding proteins that are capable of interacting with host defense factors or required for type III secretion systems that deliver virulence proteins into host cells. Usually, they are found only in pathogenic strains (or species); in rare cases, they occur in nonpathogenic strains of the same species or related species (Hacker et al., 1997, supra).

In phytopathogenic bacteria, hrp gene clusters have been referred to as “pathogenicity islands” because they have several features in common with “pathogenicity islands” in animal pathogenic bacteria, i.e., they are found only in pathogenic species (required for plant pathogenicity) and contain highly conserved genes (hrc genes) defining the type III protein secretion system (Alfano and Collmer, 1996; Barinaga, 1996).

In plant pathogenic fungi, genes or gene clusters with characteristics of “pathogenicity islands” have been identified from certain species, i.e., in Nectria haematococca, the PDA genes for detoxifying the pea phytoalexin and other pea pathogenicity genes (PEP) are located on dispensable chromosomes that are found in all isolates pathogenic to pea but usually absent in all nonpathogenic isolates (VanEtten et al., 1994; Liu et al., 1997, supra). In the genus Cochliobolus, the Tox2 gene cluster controlling the biosynthesis of HC-toxin is found only in C. carbonum race 1 (pathogenic to hm1hm1 corn) and the Tox1 genes controlling T-toxin production are found only in C. heterostrophus race T (highly virulent on T-cytoplasm corn); all other races of the same species and all other fungal species tested so far lack these Tox genes (Ahn and Walton, 1996, supra; Yang et al., 1996, supra; Yoder et al., 1997, supra).

CPS1 differs in two important ways compared to these fungal “pathogenicity islands”. First, it is highly conserved among several phytopathogenic Cochliobolus species and relatives. Second, like certain bacterial “pathogenicity islands”, CPS1 also has homologs in “nonpathogenic” species. C. homomorphus and C. dactyloctenii, neither of which causes disease on plants, hybridized strongly to CPS1. This may reflect genetic changes in the “pathogenicity island” that resulted in loss of pathogenicity. In the bacterial genus Listeria, which includes several human or animal pathogenic species harboring highly conserved “pathogenicity islands”, the “pathogenicity island” homolog in the nonpathogenic species (L. seeligeri) was found to be ‘silent’ due to a mutation that occurred in the promoter region of a critical regulatory gene in the cluster (Hacker et al., 1997, sup/a). These features suggest that the CPS1 gene cluster and homologs could define a new group of fungal “pathogenicity islands”.

The origin of CPS1. It is known that the evolution of pathogenicity involves two major processes. A pathogenic microorganism could originate from nonpathogenic progenitors by slow modifications (such as point mutations and genetic recombination) of genes that were adapted for parasitic growth on hosts or by the integration of large fragments of “alien” DNA into the genome that enable the recipient to attack particular hosts (gene horizontal transfer). The latter can occur in the recent or distant evolutionary past. Subsequent vertical transmission in the lineage (if the transferred gene is stable in the recipient genome) would result in the preserve of the gene in all species that diverged after the acquisition of the gene(s) (Scheffer, 1991; Arber, 1993; Krishnapillai, 1996; Burdon and Silk, 1997).

In the past few years, substantial evidence has become available that supports the hypothesis of gene horizontal transfer. All “pathogenicity islands” in animal pathogenic bacteria are believed to have been acquired by a horizontal transfer event (recent or past) because they usually differ in G+C content from the recipient genome and have transposable elements at the boundaries of the gene clusters (Hacker et al., 1997, supra). The hrp “pathogenicity islands” do not show a significant difference in G+C content or association with transposable elements, but they are also believed to have arisen similarly because hrc genes in these “pathogenicity islands” show high similarity to genes defining the type III protein secretion system found in animal pathogenic bacteria as mentioned above (Alfano and Collmer, 1996; and Barinaga, 1996).

Although CPS1 itself has several typical fungal introns and a G+C content (51.5%) similar to most known fungal genes, genomic regions (about 1.5 kb) flanking the gene have higher G+C content (>60%). Several short G+C-rich regions are also found in the gene cluster; one of the open reading frames (ORF10) has a 63.6% G+C content. Compared to those filamentous fungal genomes characterized so far, including N. crassa, A. nidulans, U. maydis (all have G+C content 51-54%, see Karlin and Mrázek, 1997, supra), the genomic region around CPS1 is unusual. This might suggest that the gene cluster harboring CPS1 came from a bacterial source (since most bacterial genes are known to have a high G+C content), but has evolved into a fungal version.

Based on these data, CPS1 homologs may have a common ancestral gene which was acquired from a bacterial species via horizontal transfer and then maintained by the fungal genome via vertical transmission in closely related lineages.

In the evolution process, the genus Cochliobolus could also have inherited a second gene (X) controlling the ability to take up foreign DNA, by which its ancestor took the “alien” CPS1. As a result, this group of fungi is able to keep trapping genes from other organisms by additional “horizontal transfers” and giving rise to new races or even new species characterized by the ability to produce unique pathogenesis factors. The direct support for this hypothesis is that both the Tox2 locus of C. carbonum and the Tox1 locus of C. heterostrophus are associated with large fragments of “alien” DNA (A+T-rich and highly repeated) and the same could also be true for Tox3 controlling victorin production by C. victoriae, although there is yet no direct experimental evidence (Ahn and Walton, 1996, supra; Yang et al., 1996, supra; Yoder et al., 1997, supra). In contrast to CPS1, these gene transfers must have occurred in the recent evolutionary past because both Tox1 and Tox2 loci are found only in specific isolates in the species, e.g., the acquisition of Tox1 genes probably occurred as recently as the 1960s when race T was first identified in the field (Yoder et al., 1997, supra).

There are other possibilities for the evolution of CPS1. First, each genus mentioned above could have acquired CPS1 independently after divergence of the lineage. But this seems less likely because this would need to happen at the same time and involve the same donor organism if the fact that the homologs detected in Cochliobolus and Setosphaeria gave similar hybridization signal intensity is considered. Second, the horizontal transfer of CPS1 could have occurred at earlier time periods such as before the divergence of Pleosporales or even the Ascomycotina To test these hypotheses, detection of CPS1 homologs in Pyrenophora, Pleospora and other genera must be done by either genomic DNA hybridization or PCR. Based on the facts discussed here, it is not unreasonable to predict that additional CPS1 homologs will be found in other fungal species. Further investigation could provide a direct entry point for understanding the evolution of fungal pathogenesis to plants.

### EXAMPLE 5

### Other Genes Near Cochliobolus CPS1

Materials and Methods

Construction of genomic library of C. heterostrophus. The cosmid SuperCosP1-11 (kindly provided by Dr. Thomas Hohn of Mycotoxin Research Unit USDA/ARS), which is a modification of the cosmid vector cosHyg1 (Turgeon et al., 1993, supra), was used for library construction. Genomic DNA of strain C4 (Tox⁺; MAT-2) was prepared as previously described (Yoder, 1988, supra) and purified by the equilibrium centrifugation in CsCl-ethidium bromide gradients (Sambrook, et al., 1989, supra). Three 1 g of genomic DNA was partially digested with MboI using a test series of enzyme dilutions (1.5×10⁻⁴-1.25 units, New England Biolabs, Beverly, Mass.) at 37° C. for 0.5 hour. DNA from the digestions which yielded fragments with an average size of 30 kb was pooled and then dephosphorylated with Calf Intestinal Alkaline Phosphatase (CLAP, GIBCO BRL Products, Gaithersburg, Md.). Two ì g of CIAP-treated DNA was ligated into the BamHI site of the cosmid vector that had been digested with XbaI and treated with CIAP. Aliquots of the ligated molecules were packaged using Gigapack II Packaging Extract (Stratagene, La Jolla, Calif.) according to the manufacturer's recommendations. E. coli strain NM554 was transfected with the packaged phage particles and selected for ampicillin resistance. Approximately 1.6×10⁵ independent ampicillin resistant colonies were obtained from two experiments. Cosmid DNAs were made from 16 colonies and digested with HindIII and EcoRI respectively to confirm random insertions. Colonies were scraped from each of the original LB plus ampicillin plates and stored at −70° C. in 25% glycerol (one plate of colonies/per tube).

Screening of the cosmid library. A mixture of cosmid clones from 23 stored tubes was diluted to 10⁻⁴ spread on ten LB plus ampicillin plates (150×15 mm) and incubated at 37° C. overnight. Colonies (total about 1.2×104) were transferred to Colony/Plaque Screen™ Hybridization Transfer Membrane (137 Mm discs, NEN™ Life Science Products, Boston, Mass.) and incubated at 37° C. for 8 hours. Three replicates were made of each plate (one as master filter and two for probing). For hybridization, filters carrying colonies were lysed in 0.5 N NaOH, 1.5 M NaCl for 5 minutes, neutralized twice in 1 M Tris pH 7.4, 1.5 M NaCl for 5 minutes followed by 2×SSC for 2 minutes. Filters were air dried 30 minutes then baked in a vacuum oven at 80° C. for 1 hour. Duplicate filters were probed with ³²P labeled 3.4 and 3.2 kb fragments of the CPS1 gene (cloned on p214B7 and p214S1, respectively) that were prepared by restriction enzyme digestion and purification using QLAquick Gel Extraction Kit (QIAGEN Inc., Chatsworth, Calif.). Hybridization was in 6×SSC, 1×BLOTTO (Sambrook et al., 1989) at 65° C. overnight. Then filters were then washed twice for 15 minutes, 65° C. in 2×SSC, 0.1% SDS. Cosmid clones corresponding to positive areas were transferred from the master filters into a 96-well microtiter plate (Corning Costar, Cambridge, Mass.) and allowed to grow at 37° C. overnight. Cells were then transferred onto membranes using a frogger, incubated and processed same as above. Positive clones were purified and re-tested by hybridization with the same probes as mentioned above. The isolated cosmid clones were mapped by probing cosmid DNA digested with several enzymes with the labeled 3.4 and 3.2 kb CPS1 fragments separately.

DNA manipulations and sequencing. Cosmid DNA was prepared using standard protocols (Sambrook, et al., 1989, supra). Restriction enzyme digestions, gel electrophoresis, gel blot analysis, primer design, DNA sequencing and sequence analysis were done as described above. To facilitate sequencing, three deletion constructs were made by digestion of the original cosmid clones (Table 10) with restriction enzymes that do not cut the cosmid vector, followed by religation (Table 10). Sequencing of each cosmid clone was initiated with vector-specific and CPS1 (or TES1)-specific primers. Subsequently, sequences were extended by designing new primers to the previously sequenced region (Table 11).

Results

Characterization of two overlapping cosmid clones. Two cosmid clones, C4L6582 and C4L7296, were isolated by screening the library (Table 10). Gel blot analysis indicated that both cosmid clones span the vector insertion site in the REMI mutant and contain the cloned CPS1 and TES1 sequences described above. Sequence obtained using a primer to the region immediately flanking the insertion site is the same as that in the tagged DNA recovered from the REMI mutant, confirming that no deletions or chromosome rearrangements occurred at the tagged site. Two cosmids overlap each other in a 27.9 kb region. C4L7296 (37.2 kb) carries a 30.9 kb genomic insert which hybridized to both 3.4 kb and 3.2 kb CPS1 fragments. Restriction mapping and sequencing confirmed that this insert contains the entire TES1 sequence and most of the CPS1 sequence (4.4 out of 5.4 kb). C4L6582 (37.7 kb) carries a 31.4 kb insert that also includes the entire TES1 sequence but only 1.1 kb of the N-terminal encoding sequence of CPS1. Both inserts lack the C-terminal region of CPS1; their 3′ end is ligated to the T3 end of cloning site in SuperCosP1-11. Attempts to sequence using the T7 primer were unsuccessful, presumably because the T7 end, which is close to one of the cos sites on SuperCosP1-11 was disrupted during the packaging process.

TABLE 10
Cosmid and plasmid clones used in this study

| Clones | | | |
|---|---|---|---|
| (kb) | Length | Characteristics | Reference |
| Super- | 6.9 | Cosmid vector for library construction | Horwitz et al., |
| CosP1-11 | | containing the 2.5 kb HindIII-SalI | 1997 |
| | | fragment from pH1S carrying hygB gene | |
| | | fused to C. heterostrophus promoter 1. | |
| pUCATPHN | 4.6 | Cloning vector derived from pUCATPH. | This study |
| C4L6582 | 37.7 | A cosmid clone with a 31.4 kb insert | This study |
| | | isolated from screening the library. | |
| | | Includes 4.0 kb region p214B7. | |
| C4L7296 | 37.2 | A cosmid clone with a 30.9 kb insert | This study |
| | | isolated from screening the library. | |
| | | Includes 6.3 kb region p214B7 + p214S1. | |
| p6582dH | 10.9 | A deletion (28.8 kb) construct derived | This study |
| | | from digestion of C4L6582 with HindIII. | |
| p6582dS | 21.1 | A deletion (16.6 kb) construct derived | This study |
| | | from digestion of C4L6582 with SacI. | |
| p7296dX | 9.0 | A deletion (28.2 kb) construct derived | This study |
| | | from digestion of C4L7296 with XhoI. | |
| pDXPS* | 13.6 | Ligation of 7296dX digested with XhoI | This study |
| | | to the SalI-digested pUCATPHN. | |
| pDXPSH* | 6.5 | A plasmid derived from pDXPS by HindIII | This study |
| | | digestion and religation of a 6.5 kb HindIII | |
| | | fragment containing the entire pUCATPHN | |
| | | sequence flanked by 1.2 kb of the 5′ end of | |
| | | CPS1 and 0.5 kb 3′ end of C4L7296 sequence | |

*Designed for deletion of the 28.2 kb of genomic region (= deleted from p7296dX, including 3.6 kb CPS1 N-terminal encoding sequence) but transformation of wild type was unsuccessful.

TABLE 11
Primers used for sequencing genomic DNA
on C4L7296 and C4L6582

| | | | | |
|---|---|---|---|---|
| Name^{a} | Position^{b} | Sequence^{c} | Template^{d} | Origin |

F-I

| | | | | |
|---|---|---|---|---|
| 214RP7 | | SEQ ID NO: 148 | A | p214B7 |
| 1. RP8 | 4940 | SEQ ID NO: 149 | A | 7296RP |
| 2. RP9 | 592 | SEQ ID NO: 150 | A | 7296RP8 |
| 3. RP10 | 4124 | SEQ ID NO: 151 | A | 7296RP9 |
| 4. RP11 | 3790 | SEQ ID NO: 152 | A | 7296RP10 |
| 5. RP12 | 3424 | SEQ ID NO: 153 | A | 7296RP11 |
| 6. RP13 | 2970 | SEQ ID NO: 154 | A | 7296RP12 |
| 7. RP14 | 2362 | SEQ ID NO: 155 | A | 7296RP13 |
| 8. RP15 | 1764 | SEQ ID NO: 156 | A | 7296RP14 |
| 9. RP16 | 1169 | SEQ ID NO: 157 | A | 7296RP15 |
| 10. RP17 | 647 | SEQ ID NO: 158 | A | 7296RP16 |

F-II

| | | | | |
|---|---|---|---|---|
| 214RP2 | | SEQ ID NO: 159 | B | p214B7 |
| 11. SRP1 | 3095 | SEQ ID NO: 160 | A | 6582dSRP2 |
| 12. SRP2 | 2755 | SEQ ID NO: 161 | A | 7296dSRP1 |
| 13. SRP3 | 2366 | SEQ ID NO: 162 | A | 7296dSRP2 |
| 14. SRP4 | 2008 | SEQ ID NO: 163 | A | 7296dSRP3 |
| 15. SRP5 | 1555 | SEQ ID NO: 164 | A | 7296dSRP4 |
| 16. SRP6 | 1187 | SEQ ID NO: 165 | A | 7296dSRP5 |
| 17. SRP7 | 647 | SEQ ID NO: 166 | A | 7296dSRP6 |
| 18. SFP1 | 3321 | SEQ ID NO: 167 | A | 6582dSRP2 |
| 19. SFP2 | 3660 | SEQ ID NO: 168 | A | 7296dSFP1 |
| 20. SFP3 | 3969 | SEQ ID NO: 169 | A | 7296dSFP2 |
| 21. SFP4 | 4345 | SEQ ID NO: 170 | A | 7296dSFP3 |
| 22. SFP5 | 4724 | SEQ ID NO: 171 | A | 7296dsFP4 |
| 23. SFP6 | 5137 | SEQ ID NO: 172 | A | 7296dSFP5 |
| 24. SFP7 | 694 | SEQ ID NO: 173 | A | 7296dSFP6 |

F-III

| | | | | |
|---|---|---|---|---|
| TrpC | | SEQ ID NO: 174 | C | pUCATPH |
| 214FP6 | | SEQ ID NO: 175 | D | p214S1 |
| 25. CFP1 | 463 | SEQ ID NO: 176 | A | pDXPSTrpC |
| 26. CFP2 | 903 | SEQ ID NO: 177 | A | 7296pUCFP1 |
| 27. CFP3 | 1334 | SEQ ID NO: 178 | A | 7296pUCFP2 |
| 28. CFP4 | 1910 | SEQ ID NO: 179 | A | 7296pUCFP3 |
| 29. CFP5 | 2491 | SEQ ID NO: 180 | A | 7296pUCFP4 |

F-IV

| | | | | |
|---|---|---|---|---|
| 214B7RP5 | | SEQ ID NO: 181 | E | p214B7 |
| 30. HRP1 | 592 | SEQ ID NO: 182 | F | 6582dHRP5 |
| 31. HFP1 | 763 | SEQ ID NO: 183 | F | 6582dHRP5 |

^{a}“RP” indicates reverse primer; “FP” indicates forward primer. Primers designed to genomic DNA on the cosmid clones are numbered in order. Primers 1-10 are preceded by “7296”; 11-24 by “7296d”; 25-29 by “7296pU” and 30-31 by “6582d”.
^{b}Primer position corresponds to position in the genomic sequences of each fragment.
^{c}Each primer sequence is given in the 5′ to 3′ direction.
^{d}Cosmids or plasmids used for sequencing reactions. A = C4L7296; B = 6582dS; C = pDXPS; D = pDXPSH; E = 6582dH; F = C4L6582.

Sequencing of C4L7296. A total of 27.4 kb additional genomic sequence 5′ of TES1 was cloned. Four fragments with totaling 16.9 kb (60%) were sequenced, three of which were sequenced using C4L7296 as template. Sequencing of Fragment I (F-I, 5.3 kb) began with primer 214B7RP7 (which matches the 5′ end of TES1), then was followed by sequencing with primers designed to previously determined sequences. Fragment II (F-II, 6.9 kb) was started using primers to sequences flanking the SacI site previously determined by sequencing the deletion construct 6582dS (see Table 10) and subsequently extended in both directions. Sequence of Fragment III (F-III, 3.2 kb) was obtained in a complicated manner as part of the attempt to create a deletion construct for transformation. The first part of the sequence was obtained from the clone pDXPS derived from deletion construct 7296dX (Table 10) using the TrpC primer and the sequence was extended to the 3′ end using C4L7296 as template. A 200 bp region at the 5′ end of FIII was obtained from a pDXPS derived clone, pDXPSH (Table 10), using a CPS1-specific primer 214S1FP6.

Sequencing of C4L6582. This clone contains 2.8 kb additional genomic DNA extending into the region to the left end of C4L7296. The deletion clone 6582dH (Table 10) was used to initiate sequencing of Fragment IV (F-IV, 1.5 kb) using a TES1-specific primer 214B7RP5 followed by one step of sequence extension in both 3′ and 5′ direction on C4L6582.

Identification of open reading frames in the sequenced region. Eleven open reading frames (ORF) were identified in the four sequenced fragments (Table 12). These ORFs are all relatively small (0.3-2.3 kb). Five ORFs contain putative introns with typical fungal characteristics (Table 13). ORF12, ORF10, ORF14, ORF5 and ORF8 are transcribed in one direction; others are transcribed in the opposite direction. ORF6 and ORF7 (in F-II) overlap and are transcribed in the same direction. ORF14 and ORF9 (in F-1), ORF3 and ORF8 (in F-I) also overlap but are transcribed to the opposite directions. Most ORFs have G+C content between 50-55% in the normal range for most fungal genes with the two exceptions: ORF (0.3 kb) in the 5′ end of F-III has a G+C content of 63.6%; ORF14 (0.7 kb, located 1.0 kb downstream of ORF10) has a G+C content 56.9%. Both ORFs are located in a G+C-rich (about 58.0%) region in F-III (positions 300-800 and 1240-2040, respectively).

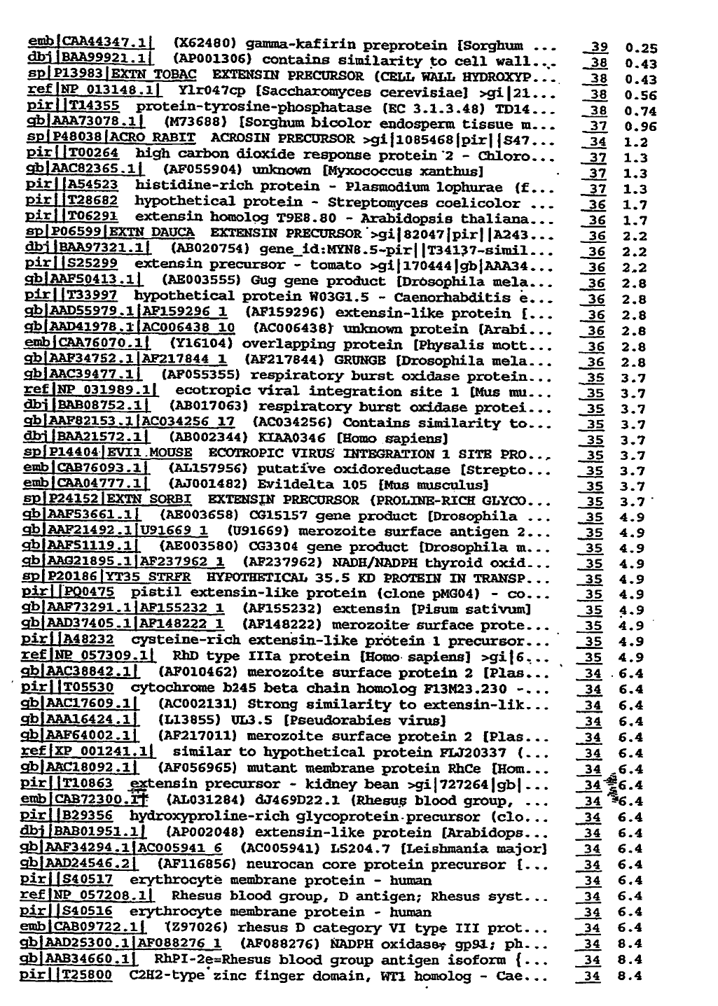

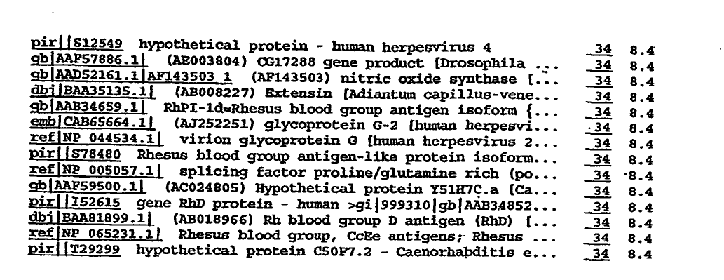

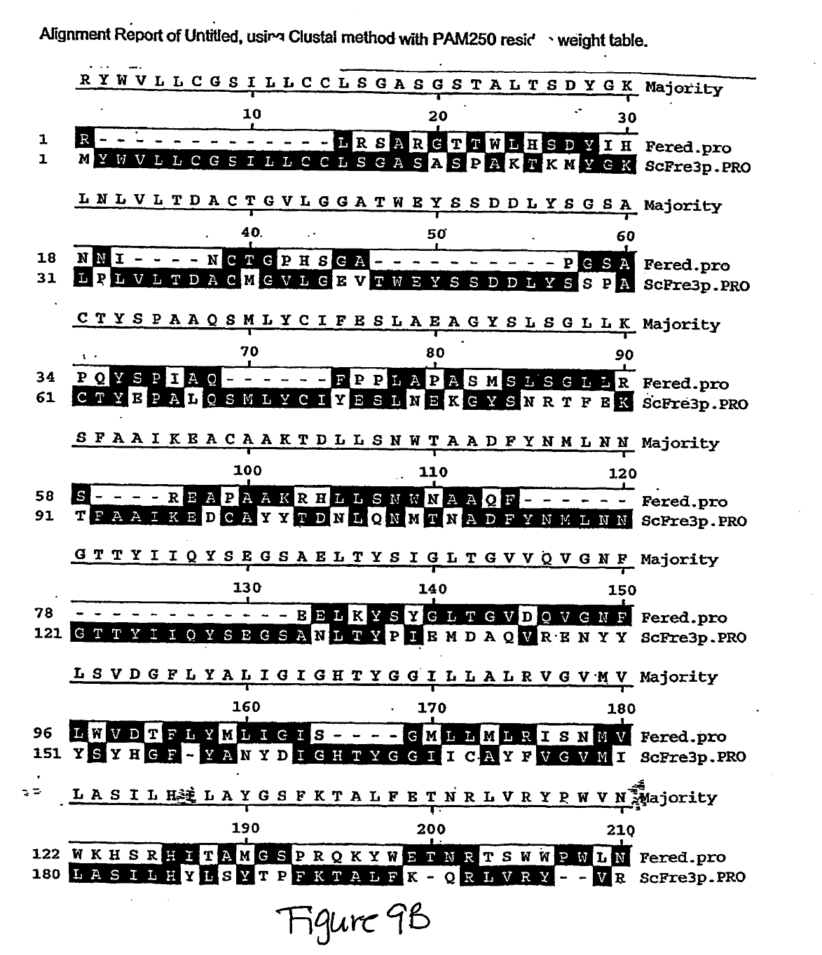

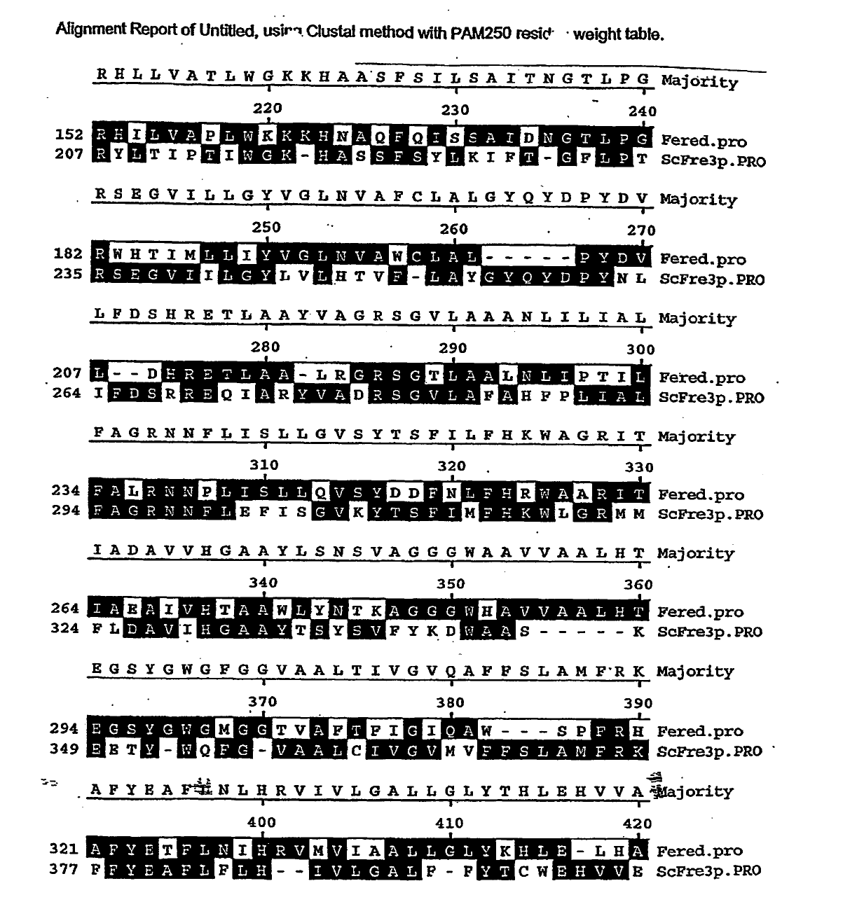

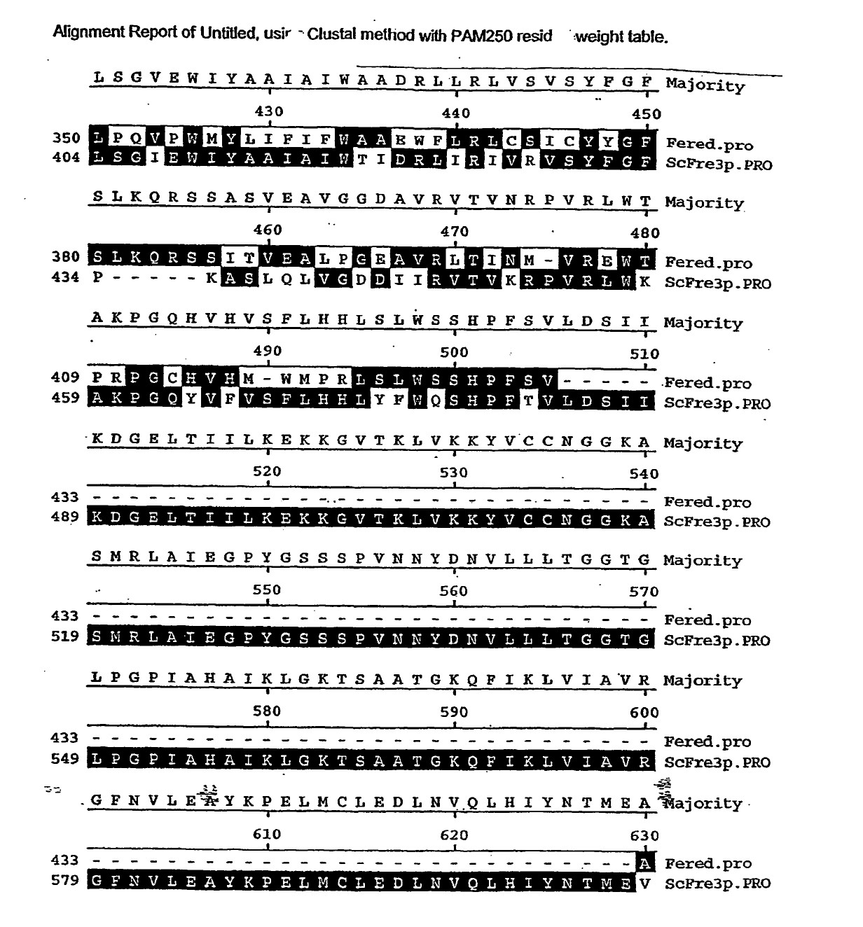

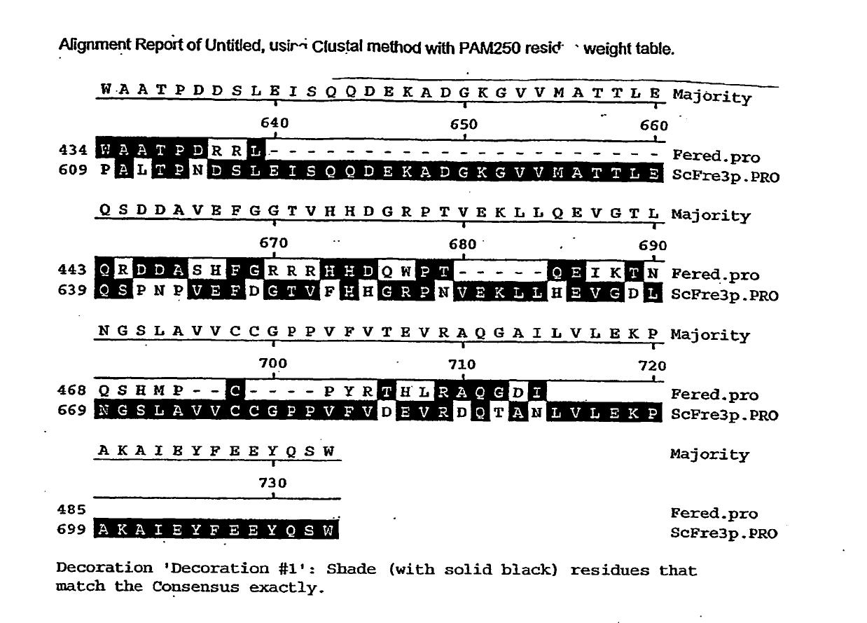

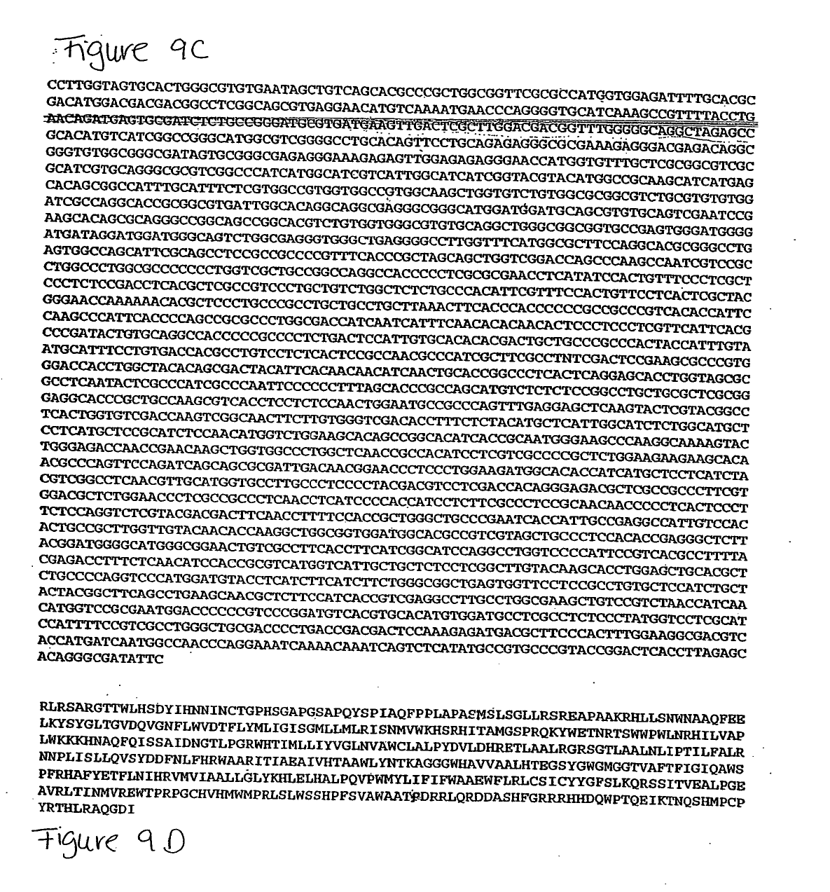

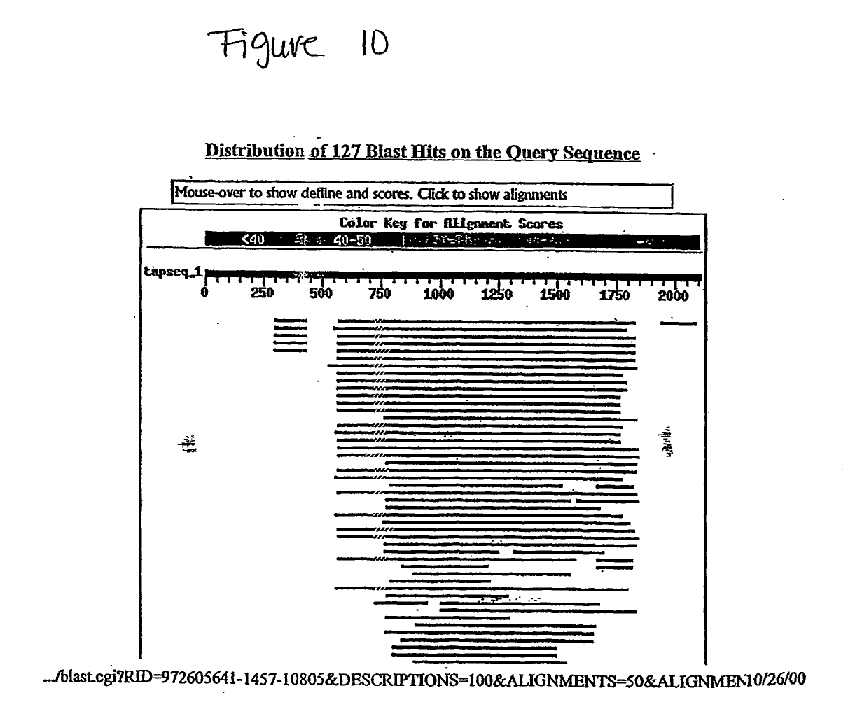

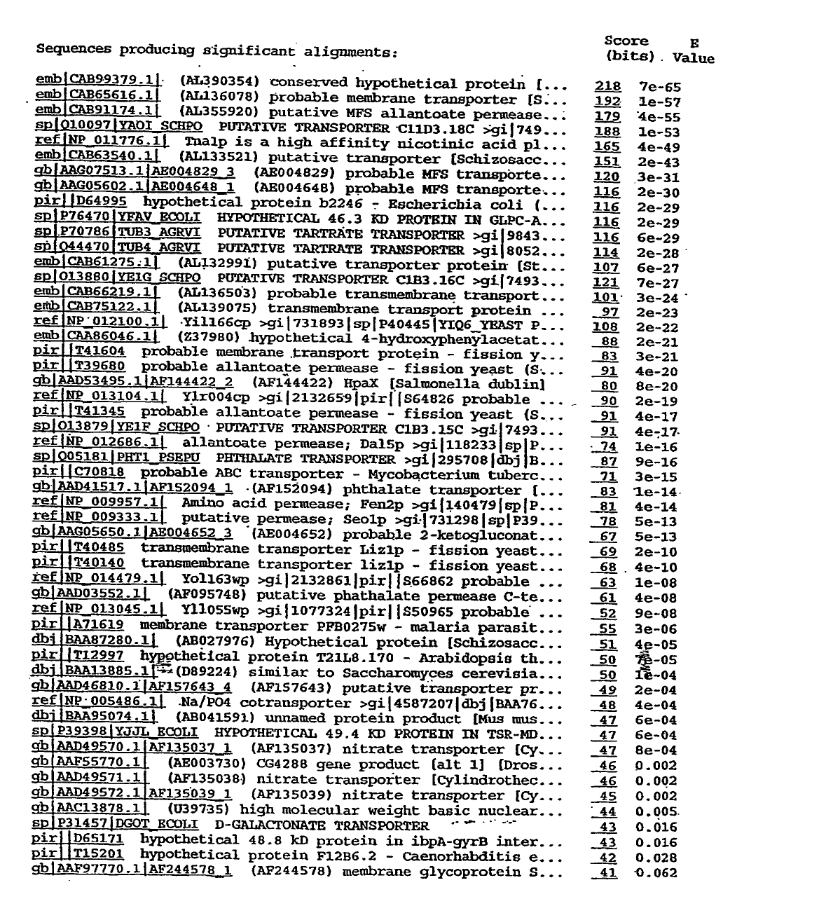

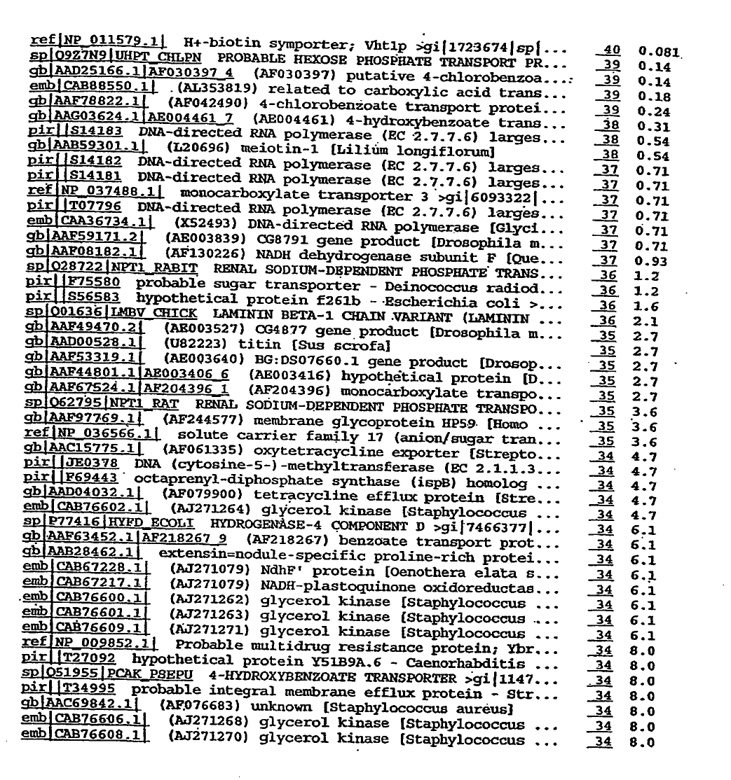

Database searches suggested that three ORFs (ORF3, ORF7 and ORF11) as well as CPS1 and TES1 encode homologs of known proteins (see below) and others encode, if anything, proteins with unknown functions (Table 12). ORF 17 (SEQ ID NO:48) encodes an iron reductase (SEQ ID NO:49) and ORF15 (SEQ ID NO:55) encodes a permease/MFS transporter (SEQ ID NO:56). FIG. 9A shows the results of a BLAST search with SEQ ID NO:49 and FIG. 10 shows the results of a BLAST search with the polypeptide encoded by SEQ ID NO:55.

TABLE 12
Open reading frames (ORFs) identified in sequenced genomic regions
of C4L7296 and C4L6582

| | | | No. of | | Putative |
|---|---|---|---|---|---|
| Region^{a} | ORF^{b} | Size (kb) | introns | G + C (%) | Function |
| F-I′ | ORF1^{d} | 5.4 | 3 | 51.5 | Peptide synthetase |
| F-I′ | ORF2^{d} | 1.1 | 1 | 55.5 | Thioesterase |
| F-I | ORF3 | 1.8 | 3 | 50.0 | DNA-binding |
| F-I | ORF8 | 0.5 | 0 | 55.2 | unknown |
| F-I | ORF11 | 1.9 | 0 | 52.6 | CoA transferase |
| F-II | ORF5 | 2.3 | 1 | 54.1 | unknown |
| F-II | ORF6 | 0.5 | 0 | 51.6 | unknown |
| F-II | ORF7 | 1.7 | 1 | 52.0 | Decarboxylase |
| F-III | ORF9 | 0.7 | 0 | 54.2 | unknown |
| F-III | ORF10 | 0.3 | 0 | 63.6 | unknown |
| F-III | ORF13 | 0.8 | 1 | 53.6 | unknown |
| F-III | ORF14 | 0.7 | 0 | 56.9 | unknown |
| F-IV | ORF12 | 1.2 | 1 | 49.2 | unknown |

^{a}F-I′ = Genomic DNA
^{b}The positions of ORF3-ORF14 and 17 in the sequenced fragment is indicated; ORFs corresponding to known proteins are underlined.
^{c}The characteristics of putative introns are given in Table 12.
^{d}Characterized as CPS1 and TES1

TABLE 13
Characteristics of putative introns in ORFs
identified in sequenced genomic regions on cosmids
C4L7296 and C4L6582

| | In- | Size | | | 3′ | Branch |
|---|---|---|---|---|---|---|
| ORF | tron | (bp) | Location^{a} | 5′Border | Border | site |
| ORF3 | I | 64 FI | 5094-5031 | GTACGT | TAG | CGCTGAC |
| | II | 46 FI | 5006-4961 | GTGAGT | TAG | AGCTAAG |
| | III | 46 FI | 4477-4432 | GTACGT | CAG | AGCTGAC |
| ORF5 | I | 48 FII | 3477-3524 | GTATGT | TAG | TGCTAAC |
| ORF7 | I | 114 | 2307-2194 | GTGTGC | CAG | ATCTAAC |
| | | FII | | | | |
| ORF13 | I | 51 | 2742-2692 | GTGCGT | CAG | TACTGAT |
| | | FIII | | | | |
| ORF12 | I | 47 FIV | 1007-1053 | GTAAGT | TAG | GATTGAC |
| Con- | | | | GT^{A}/_{G}YGT | ^{T}/_{C}AG | NRCTAAC^{b} |
| sensus | | | | | | |

^{a}Number of the fragment followed by the position of the first and last nucleotide of the intron with respect to the total sequence.
^{b}Y = Pyrimidine (T or C); R = purine; N = purine or pyrimidine.

Discussion

Two cosmids define a large ne cluster. The C. heterostrophus CPS1 gene was cloned by identification of genomic DNA fragments recovered from the tagged site in a mutant generated using REMI insertional mutagenesis. Characterization of two overlapping cosmid clones in this study has proved that no deletions or chromosome rearrangements are associated with the gene tagging event, because both cosmids carry the same fragment which span the REMI insertion site and the nucleotide sequence in this region is the same as that of recovered genomic DNA from the tagged site. This undoubtedly clarifies the identity of CPS1, which is the major biosynthetic gene. Mapping and sequencing of the two cosmids extended the sequence by 27.4 kb from the previously cloned fragment, leading to the characterization of 38.7 kb of contiguous genomic DNA, the largest genomic region analyzed so far in C heterostrophus. In addition to CPS1 and TES1, sequence analysis of this region revealed at least 11 open reading frames; three of them, designated as DBZ1, CAT1 and DEC2, respectively, apparently encode functional proteins (Table 13). The tight linkage of these genes suggests that they may be involved in the same pathway.

In filamentous fungi, in some cases, genes in pathways for biosynthesis of secondary metabolites are dispersed on different chromosomes, e.g., the cephalosporin C pathway genes in Acremonium chrysogenum (Mathison et al., 1993, supra) and the melanin pathway genes in Colletotrichum lagenarium (Kubo et al., 1996, supra). In other cases, tightly linked genes are usually found to be functionally related to a common pathway. This clustering organization has been exemplified by the sterigmatocystin pathway genes of Aspergillus nidulans, in which 25 coordinately regulated transcripts are found in a 60 kb genomic region (Brown et al., 1996) and the trichothecene pathway genes of Fusarium sporotrichioides, in which 9 genes are clustered in a 25 kb region and 8 of them have been shown to be required for the pathway function (Hohn et al., 1995). The genes involved in biosynthesis of certain fungal peptides are also found as clusters. The tight linkage between CPS1 and these additional genes might reveal the presence of a novel secondary metabolite pathway in C. heterostrophus. In this pathway, CPS1 is the major structural gene since it encodes a large multifunctional enzyme with all catalytic activities required for synthesis of a secondary metabolite, presumably a peptide phytotoxin; other genes may carry out different functions required for coordinate operation of the pathway, such as regulation, posttranslational modification or substrate processing as discussed below.

Significance of the CPS1 gene cluster. Both functional and structural analyses strongly support the hypothesis that the CPS1 gene cluster controls a novel biosynthetic pathway. Pathway genes have been studied only in a few filamentous fungi mainly for industrial purposes (Keller et al., 1997, supra). For plant pathogenic fungi, little is known about pathway genes for fungal pathogenesis. In C. heterostrophus, recent cloning of two Tox1 genes PKS1 (Yang et al., 1996, supra) and DEC1 (Rose et al., 1996, supra) have contributed to a breakthrough in understanding the molecular mechanism for biosynthesis of T-toxin, a virulence determinant in the fungus/corn interaction. But further identification of related pathway genes has been unsuccessful because the two genes are located on different chromosomes and each is embedded in A+T-rich DNA (Yoder et al., 1997, supra). In contrast, the CPS1 cluster provides a good opportunity to explore a pathogenesis pathway.

First, it resides in a “normal” sequence region. G+C content of a 50-55% is found in most of the cloned sequences and no A+T-rich DNA is associated with either end of the cloned region. This would facilitate cloning of additional pathway genes by further chromosome walking, by screening of cosmid libraries or the targeted integration and plasmid rescue. Second, it contains a regulatory gene (DBZ1) which is presumably linked to a signal transduction pathway. Isolation of genes that interact with DBZ1 could reveal novel factors mediating the molecular communication between fungal pathogen and the host plant. Further characterization of DBZ1 (along with position-specific disruption or deletion) would be also helpful in determining the limit of the gene cluster, because tightly linked genes involved in a common pathway are often coordinately regulated by the same regulatory factor (Keller et al., 1997, supra). Finally, CPS1 genes are found in both race T and race O, and its homologs are also found in other Cochliobolus species. Presence of high G+C content may imply that these genes evolved from a bacterial ancestor and the conservation in these fungi may correlate with the phytopathogenic function of the gene products encoded by the CPS1 cluster. Further investigation of this cluster should provide insights into the evolution of general pathogenicity factors among this group of fungi.

ORF17 is an iron reductase (SEQ ID NO:49) and ORF15 is a permease/MFS transporter (SEQ ID NO:56). Ferric reductases are a group of enzymes found in bacteria, fungi, plants and animals that are responsible for reduction of ferric iron to ferrous iron, an absorptive form used by the organism. They have been well studied in S. cervisiae, C. albicans and H. capsulatum and the like. The yeast FER1 has been expressed in tobacco (Oki et al., 1999).

Previous studies have shown that FER genes could be important pathogenic determinants. Timmerman and Woods have proposed that in H. capsulatum FER could play critical roles in the acquisition of iron in three different ways: from inorganic or organic ferric salts, from host Fe(III) binding proteins (transferrin and the like), and from siderophores produced by the fungus itself (to reduce and release the iron chelated by the siderophore molecules).

On the other hand, iron sequestration in response to microbial infection has been demonstrated to be a host defense mechanism. The infection-related iron acquisition system in the pathogen can be considered to be an important mechanism against host defense and for a successful colonization by the pathogen in the host cells. This could be a general mechanism for all pathogenic fungi.

CPS1 may encode an enzyme which is responsible for biosynthesis of a novel siderophore with unusual amino acid, hydroxyl acid and architecture. The CPS1 siderophore can compete with the host for iron acquisition when the fungus enters its host cells where the iron is limited due to host sequestration. In particular, for root pathogens such as C. victoriae, sequestration may be stronger in the root surface. This could explain why the cps1 mutant showed drastically reduced virulence. The FER1 could be required to release iron from the CPS1 siderophore which explains its location near the CPS1 gene. Moreover, fungal strains could be cultured in iron-limiting conditions because CPS1, and likely other genes in the cluster maybe turned on only during conditions of iron depletion.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. An isolated polynucleotide comprising a fungal nucleic acid segment which encodes a polypeptide which is substantially similar to a polypeptide encoded by a nucleic acid sequence comprising an open reading frame comprising SEQ ID NO:46, SEQ ID NO:48, or SEQ ID NO:55, or the complement thereof.

2. An isolated polynucleotide comprising a fungal nucleic acid segment which is substantially similar to a nucleic acid sequence comprising an open reading frame comprising SEQ ID NO:46, SEQ ID NO:48, SEQ ID NO:55, or the complement thereof.

3. An isolated polynucleotide comprising a fungal nucleic acid segment which hybridizes under stringent hybridization conditions to SEQ ID NO:46, SEQ ID NO:48, SEQ ID NO:55, or the complement thereof.

4. The isolated polynucleotide of claim 1, 2 or 3 which consists of SEQ ID NO:46, SEQ ID NO:48 or SEQ ID NO:55 of the complement thereof.

5. The isolated polynucleotide of claim 1, 2 or 3 wherein the nucleic acid segment is from Ascomycota.

6. The isolated polynucleotide of claim 1, 2 or 3 wherein the nucleic acid segment is from a pathogenic fungus.

7. The isolated polynucleotide of claim 1 wherein the nucleic acid segment encodes a polypeptide having at least 80% identity to a polypeptide comprising SEQ ID NO:47, SEQ ID NO:49 or SEQ ID NO:56.

8. The isolated polynucleotide of claim 1 wherein the nucleic acid segment encodes a polypeptide having at least 90% identity to a polypeptide comprising SEQ ID NO:47, SEQ ID NO:49 or SEQ ID NO:56.

9. An isolated polypeptide encoded by the polynucleotide of any one of claims 1 to 8.

10. An expression cassette comprising a promoter operably linked to the polynucleotide of any one of claims 1 to 8.

11. A recombinant vector comprising the polynucleotide of any one of claims 1 to 8 wherein the vector is capable of being stably transformed into a host cell.

12. The vector of claim 11 wherein the polynucleotide is operably linked to a promoter operable in a eukaryotic host cell.

13. The expression cassette of claim 10 or vector of claim 11 wherein the polynucleotide is in sense orientation.

14. The expression cassette of claim 10 or vector of claim 11 wherein the polynucleotide is in antisense orientation.

15. The vector of claim 11 wherein the polynucleotide is operably linked to a promoter operable in a prokaryotic host cell.

16. A host cell comprising the expression cassette of claim 10.

17. A host cell comprising the vector of claim 11.

18. The host cell of claim 16 or 17 which is selected from the group consisting of bacteria, yeast, plant and mammal.

19. A method for identifying an agent having fungicidal or mycocidal activity, comprising:
a) contacting a fungus with an agent that binds to the polypeptide of claim 9; and
b) identifying an agent having fungicidal or mycocidal activity.

20. An agent identified by the method of claim 19.

21. A method for identifying an inhibitor of a polypeptide, comprising:
a) contacting a host cell which expresses a polypeptide encoded by the polynucleotide of any one of claims 1 to 8 with an agent; and
b) identifying an agent that inhibits the activity of the polypeptide.

22. An agent identified by the method of claim 21.

23. A method of inhibiting the growth or pathogenicity of a fungus, comprising contacting the fungus with the agent of claim 20 or 22 in an amount sufficient to inhibit the growth or pathogenicity of the fungus.

24. A method for identifying an agent having fungicidal or mycocidal activity, comprising:
a) contacting a fungus with an agent that inhibits the activity of the polypeptide of claim 9; and
b) identifying an agent having fungicidal or mycocidal activity.

25. A method for identifying an agent that modulates a polypeptide associated with pathogenicity of a fungus, comprising:
a) contacting a fungus with an agent that binds the polypeptide of claim 9; and
b) identifying an agent that modulates the pathogenicity of the fungus.

26. A method for identifying an agent that modulates the pathogenicity of a fungus, comprising:
a) contacting a fungus with an agent that inhibits the activity of the polypeptide of claim 9; and
b) identifying an agent that modulates the pathogenicity of the fungus

27. A method of identifying agents that alter the phenotype of a fungal pathogen or mycogen, comprising:
a) contacting an agent to be tested with one or more cells of a fungal pathogen or mycogen which comprises a nucleotide sequence encoding a polypeptide that is substantially similar to SEQ ID NO:47, SEQ ID NO:49, or SEQ ID NO:56; and
b) detecting or determining whether the agent selectively modulates expression or function or metabolic pathways associated with the polypeptide, thereby altering a phenotype of the cells relative to cells not contacted with the agent.

28. The method of claim 27 wherein the polypeptide is associated with virulence or pathogenicity.

29. The method of claim 27 wherein the agent alters the activity of the polypeptide.

30. The method of claim 27 further comprising identifying an agent having fungicidal, mycocidal or anti-pathogenic activity.

31. The method of claim 27 wherein cellular growth is detected or determined.

32. The method of claim 27 wherein the activity of the polypeptide is detected or determined.

33. The method of claim 27 wherein virulence is detected or determined.

34. The method of claim 27 wherein the pathogen expresses the polypeptide.

35. The method of claim 27 wherein the pathogen does not express the polypeptide.

36. A method of identifying agents that alter the phenotype of a fungal pathogen or mycogen, comprising
a) contacting an agent to be tested with one or more cells of a fungal pathogen or mycogen wherein the cells have a mutation in a nucleic acid sequence corresponding to the polynucleotide according to any one of claims 1 to 8 which mutation results in overexpression or underexpression of the encoded polypeptide;
b) detecting or determining whether the agent selectively modulates expression or function or metabolic pathways associated with the polypeptide, thereby altering a phenotype of the cells relative to one or more wild type cells not contacted with the agent.

37. The method of claim 27 or 36 wherein the pathway is associated with the production of a toxin or siderophore.

38. The method of claim 27 or 36 wherein the pathway is associated with iron metabolism, uptake or absorption.

39. The method of claim 27 or 36 wherein the pathway is associated with growth, virulence or pathogenicity.

40. An isolated antibody which specifically binds to the polypeptide of claim 9.

41. The antibody of claim 40 which is a monoclonal antibody.

42. The antibody of claim 40 which is a polyclonal antibody.

43. The method of claim 19, 23, 24, 25, 26, 27 or 36 wherein the fungus is a recombinant fungus.

44. The method of claim 43 wherein the fungus comprises a recombinant DNA molecule which encodes the polypeptide.

45. The method of claim 44 wherein the recombinant DNA molecule is overexpressed.

46. The method of claim 44 wherein the fungus comprises an antisense recombinant DNA molecule for the polypeptide.

47. The method of claim 44 wherein the genome of the fungus is disrupted so that the endogenous gene which encodes the polypeptide is not expressed.

48. A therapeutic method comprising: administering to an animal suspected of being infected with a fungal pathogen an effective amount of the agent of claim 19 or 22.

49. A method to prevent or inhibit infection of an animal or plant by a fungal pathogen, comprising: administering to the animal or plant an effective amount of the agent of claim 19 or 22 for a time and under conditions sufficient to inhibit or prevent fungal growth or reproduction.

50. The method of claim 51 or 52 wherein the animal is a human.

51. The method of claim 51 or 52 wherein the agent is topically administered.

52. A nucleic acid sequence of a polynucleotide of any one of claims 1 to 8.

53. The nucleic acid sequence of claim 52 which is stored on a computer readable medium.

54. An amino acid sequence of a polypeptide of claim 9.

55. The amino acid sequence of claim 54 which is stored on a computer readable medium.

56. The method of claim 48 or 49 wherein the animal is immunocompromised.

57. The method of claim 48 or 49 wherein the animal has Coccidioidomycosis.

58. The method of claim 48 or 49 wherein the animal is subjected to immunosuppressive therapy.

59. The method of claim 48 or 49 wherein fungal iron metabolism is inhibited.

60. The method of claim 49 wherein the agent is administered to a plant.

61. The method of claim 60 wherein the agent is administered by spraying.

62. A transformed plant, the genome of which expresses a chimeric DNA molecule which encodes a gene product which confers resistance or tolerance to the plant to a fungal pathogen by inhibiting fungal iron metabolism or siderophore production.
